(19) United States
(12) Patent Application Publication
(10) Pub. No.: US 2023/0365956 A1
(43) Pub. Date: Nov. 16, 2023

(54) RECOMBINANT ACE2-FC FUSION MOLECULES AND METHODS OF MAKING AND USING THEREOF

(71) Applicants: SYSTIMMUNE, INC., Redmond, WA (US); BAILI-BIO (CHENGDU) PHARMACEUTICAL CO., LTD., Chengdu, Sichuan (CN)

(72) Inventors: Tsung-I TSAI, Redmond, WA (US); Dennis R. GOULET, Redmond, WA (US); Nga Sze Amanda MAK, Mukilteo, WA (US); Andrew WAIGHT, Kenmore, WA (US); Steven K. LUNDY, Lake Forest Park, WA (US); Mark Gilchrist, Lake Forest park, WA (US); Jahan KHALILI, Kirkland, WA (US); Sa XIAO, Chengdu, Sichuan (CN); Muran DING, Chengdu, Sichuan (CN); Yong ZHANG, Chengdu, Sichuan (CN); Shi ZHUO, Chengdu, Sichuan (CN); Hai ZHU, Bothell, WA (US); Yi ZHU, Chengdu, Sichuan (CN)

(21) Appl. No.: 18/029,043

(22) PCT Filed: Oct. 1, 2021
(86) PCT No.: PCT/US2021/053052

§ 371 Date: Mar. 28, 2023

Related U.S. Application Data
(60) Provisional application No. 63/086,593, filed on Oct. 1, 2020.

(51) Int. Cl. C12N 9/48; A61P 31/14

(52) U.S. Cl. CPC C12N 9/485; A61P 31/14; C12Y 304/17023; C07K 2319/30

(57) ABSTRACT

A method of preventing, reducing a risk of, or treating a virus infection, or preventing or treating a symptom caused by the virus in a subject, said method comprising administering to said subject an effective amount of a fusion protein, wherein the fusion protein comprises a variant angiotensin converting enzyme 2 (ACE2) domain covalently fused to a Fc domain. The variant ACE2 domain comprises a N-terminal deletion, a C-terminal deletion, or both, relative to a full-length wildtype ACE2 having a SEQ ID NO. 1, and the variant ACE2 domain has ACE2 activity. The virus may be SARS-CoV, SARS-CoV-2, or MERS-CoV. The symptom comprises Severe Acute Respiratory Syndrome (SARS), Middle East Respiratory Syndrome (MERS), Acute Respiratory Distress Syndrome (ARDS), Pulmonary Arterial Hypertension (PAH), or Coronavirus Disease 2019 (COVID-19).

### CROSS REFERENCE TO RELATED APPLICATIONS

This application claims the benefit of the filing date of U.S. Provisional Application Ser. No. 63/086,593 filed Oct. 1, 2020 under 35 U.S.C. 119(e), the entire disclosures of which are incorporated by reference herein.

### TECHNICAL FIELD

The present application relates to the prevention or treatment of the diseases, symptoms or conditions involving Angiotensin-Converting Enzyme 2 (ACE2) such as coronavirus disease 2019 (COVID-19) and related conditions.

### BACKGROUND

Unless otherwise indicated herein, the materials described in this section are not prior art to the claims in this application and are not admitted to be prior art by inclusion in this section.

COVID-19 is an infectious disease caused by severe acute respiratory syndrome (SARS) coronavirus 2 (SARS-CoV-2). Complications of COVID-19 may include long-term lung damage, pneumonia, acute respiratory distress syndrome (ARDS), peripheral and olfactory nerve damage, multi-organ failure, septic shock, and death. A study of the first 41 cases of confirmed COVID-19, published in January 2020 in The Lancet, reported the earliest date of onset of symptoms as Dec. 1, 2019. By Mar. 11, 2020, the World Health Organization (WHO) declared the COVID-19 outbreak a pandemic. As of Sep. 26, 2020, more than 32.6 million cases have been reported across 188 countries and territories with more than 990,000 deaths, of which more than 7.5 million cases and 205,000 deaths were reported by the United States.

On 2 Dec. 2020, the United Kingdom’s Medicines and Healthcare products Regulatory Agency (MHRA) gave temporary regulatory approval for the Pfizer-BioNTech mRNA vaccine, becoming the first country to approve the vaccine and the first country in the Western world to approve the use of any COVID-19 vaccine. Since then, more types of vaccines have been authorized by at least one national regulatory authority for public use: two RNA vaccines from Pfizer-BioNTech and Moderna; three conventional inactivated vaccines from Sinopharm, Bharat Biotech, and Sinovac; three viral vector vaccines from Sputnik V, Oxford-AstraZeneca, and Janssen; and one peptide vaccine (EpiVacCorona).

As of Aug. 16, 2021, the CDC’s vaccine effectiveness studies provide growing evidence that the available RNA COVID-19 vaccines protect as well in real-world conditions as they have in clinical trial settings. The vaccines reduce the risk of COVID-19, especially severe illness, among people who are fully vaccinated. In comparison with fully vaccinated people, a study in the state of Washington found that unvaccinated people were six times more likely to test positive for COVID-19, 37 times more likely to be hospitalized, and 67 times more likely to die, compared to those who had been vaccinated. The CDC’s data show that unvaccinated people were 5 times more likely to be infected, 10 times more likely to be hospitalized, and 11 times more likely to die.

Angiotensin-converting enzyme 2 (ACE2) is a zinc-containing metalloenzyme located on the cell membrane of mainly alveolar cells of the lung, enterocytes of the small intestine, endothelial cells of arterial and venous, smooth muscle cells of arteries, and other lineages of cells in the lungs, arteries, heart, kidney, intestines, and other tissues. ACE2 regulates the renin angiotensin system by counterbalancing angiotensin-converting enzyme activity in the cardiovascular, renal and respiratory systems, indicating its important role in the control of blood pressure. ACE2 plays a protective role in the physiology of hypertension, cardiac function, heart function, and diabetes. In the acute respiratory distress syndrome (ARDS), ACE, Angll, and AT1R promote the disease pathogenesis, whereas ACE2 and AT2R protect from ARDS. In addition, ACE2 has been identified as a receptor of severe acute respiratory syndrome (SARS) coronavirus and plays a key role in severe acute respiratory syndrome (SARS) pathogenesis. Of a family of coronaviruses, at least three viruses, SARS-CoV, MERS CoV, and SARS-CoV-2, use one of their viral proteins, also known as Spike, to bind to the ACE2 protein on the surface of human host cells for the viral entry into human cells.

SARS-CoV-2 is one of seven known coronaviruses to infect humans, including SARS-CoV-1 and MERS CoV viruses that caused the outbreak of SARS in Asia in 2003 and in Middle East in 2012. The immune response to SARS-CoV-2 virus involves a combination of the cell-mediated immunity and antibody production. Although more than 100 million people have recovered from COVID-19 (as of January, 2021), it remains unknown if the natural immunity to SARS-CoV-2 virus will be long-lasting in individuals. One of the concerns relates to the virus’s continual accumulation of mutations, which may alter the spectrum of viral antigenicity and cause reinfection by mutant strains of the virus. As of January 2021, variant strains of SARS-CoV-2 virus identified in Europe and South Africa seem to be spreading so quickly. These variant strains may harbor mutations that ultimately enhance viral recognition and infection into host cells, thereby increasing infectivity and/or pathogenicity.

The other concern relates the phenomenon of antibody-dependent enhancement (ADE). ADE occurs when the binding of suboptimal antibodies enhances viral entry into host cells. In coronaviruses, antibodies targeting the viral spike (S) glycoprotein promote ADE (Wan et al., J. Virol. 2020). In cases of SARS-CoV-1 viruses, the antibodies that neutralized most variants were found to be able to enhance immune cell entry of the mutant virus, which, in turn, worsen the disease the vaccine was designed to protect against. Therefore, ADE can hamper vaccine development, as a vaccine may cause the production of suboptimal antibodies. In this context, any preventive strategy other than vaccines shall be considered as a viable alternative circumventing ADE, either before or after exposure to SARS-CoV-2 virus.

Early in the pandemic, there were few ‘mutant’ variant viruses because of the small number of people infected, thereby fewer opportunities for escape mutants to emerge. As time went on, SARS-CoV-2 started evolving to many variants and become more transmissible. Several SARS-CoV-2 variants are of particular importance due to their potential for increased transmissibility, increased virulence, or reduced effectiveness of vaccines against them (Planas et al., Nature, 2020; Kim et al., bioRxiv, 2021). To classify SARS-CoV-2 variants, the ancestral type is type “A”, and the derived type is type “B”. The B-type mutated into further types including B.1, which is the ancestor of the major global variants of concern. WHO has named Alpha (B.1.1.7, December, 2020), Beta (B.1.351, January, 2021), Gamma (P.1, January, 2021), Kappa (B.1.617.1), Delta (B.1.617.2, May, 2021), Lambda (C.37), and other variants. Both Alpha variant and the Delta variant are notably more transmissible than the original virus identified early 2020.

The Delta variant is about 40% more contagious than the alpha variant, and became the dominant strain during the spring of 2021. By late August 2021, the Delta variant accounted for 99% of U.S. cases and was found to double the risk of severe illness and hospitalization for those not yet vaccinated, and even vaccine protection by RNA vaccines fell from 91% to 66%. The CDC studies show that the COVID-19 vaccines provided 55% protection against infection, 80% against symptomatic infection, and at least 90% against hospitalization. Recent studies have demonstrated reduced vaccine efficacy of 53.1%, 42-76%, or 64.6%, with the decrease likely due to waning immunity combined with inferior protection against the highly infectious Delta strain (Nanduri, et al., MMWR. 2021; Puranik et al., medRxiv, 2021; Seppala et al., Eurosurveillance, 2021). The CDC also reported 5,814 breakthrough infections (i.e. a vaccinated individual becomes sick from the same illness that the vaccine is meant to prevent) and 74 deaths among the more than 75 million fully vaccinated people in the United States. The rate of breakthrough infections and related death may be very low, demonstrating the effectiveness of vaccines. On the other hand, breakthrough infections are likely to occur more frequently for novel strains of the virus such as Delta, as demonstrated in Israel, where over half of cases and hospitalizations in August 2021 occurred in fully vaccinated individuals (Wadman, M. Science 2021). To those individual patients who harbor breakthrough infections, particularly those frail, older adults, the risk of severe illness, delirium, hospitalization, and death is significantly high (Antonelli, et al., Lancet, 2021).

Two of the primary medical interventions for mitigating pathogenicity of SARS-CoV-2 include active and passive immunization; namely, vaccination, monoclonal antibody therapy, and treatment with convalescent plasma from previously infected patients (Taylor et al., Nat Rev Immunol., 2021; Yan et al., Pharmaceuticals. 2021). Each of these strategies relies on antibody binding and neutralization of viral antigens, in particular the receptor binding domain of the spike protein, which mediates viral entry into host cells bearing ACE2 receptors. Any viral mutations that impact the structure of the spike protein could impact the ability of antibodies to bind and neutralize spike, thus reducing the efficacy of most existing vaccines and therapeutics.

Therefore, there remains a significant need for effective treatments or preventions of the diseases or conditions involving Angiotensin-Converting Enzyme 2 (ACE2) such as SARS-CoV2 and especially its more virulent mutation strains.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

The application provides, among others, methods for preventing, reducing a risk of, or treating a virus infection, or preventing or treating a symptom caused by the virus in a subject.

In one embodiment, the virus may be a corona virus. In one embodiment, the virus may a SARS-CoV, SARS-CoV-2, MERS-CoV, or a combination thereof.

In one embodiment, the symptom may be any symptoms caused by a corona virus. In one embodiment, the symptom may be Severe Acute Respiratory Syndrome (SARS), Middle East Respiratory Syndrome (MERS), Acute Respiratory Distress Syndrome (ARDS), Coronavirus Disease 2019 (COVID-19), or a combination thereof. In one embodiment, the symptom (disease or conditions) involves Angiotensin-Converting Enzyme 2 (ACE2). In one embodiment, the symptom may be a viral infection such as an infection of SARS-CoV-2, SARS-CoV, SARS Spike protein, coronavirus, SARS virus, or a fragment or a combination thereof.

In one embodiment, the SARS-CoV-2 virus comprises substantially delta strain. In one embodiment, the SARS-CoV-2 virus comprises a Spike protein mutation. In one embodiment, the mutation is configured to increase the binding affinity of the virus to the ACE2 domain.

In one embodiment, the method includes the step of administering to the subject an effective amount of a fusion protein or a fusion protein complex. In one embodiment, the fusion protein comprises a variant angiotensin converting enzyme 2 (ACE2) domain covalently fused to a Fc domain. The variant ACE2 domain may comprise a N-terminal deletion, a C-terminal deletion, or both, relative to a full-length wild type ACE2 having a SEQ ID NO. 1, and the variant ACE2 domain has ACE2 activity.

In one embodiment, the fusion protein includes a variant angiotensin converting enzyme 2 (ACE2) domain covalently fused to a Fc domain. In one embodiment, the variant ACE2 domain comprises a N-terminal deletion, a C-terminal deletion, or both, relative to a full-length wildtype ACE2. In one embodiment, the full-length wildtype ACE2 domain has an amino acid sequence with at least 70%, 80%, 90%, 95%, 97%, or 98% sequence identity to SEQ ID NO. 1. In one embodiment, the variant ACE2 domain has ACE2 activity.

In one embodiment, the variant ACE2 domain comprises an amino acid sequence having at least 75%, 80%, 85%, 90%, 95%, 97%, 98%, or 99% sequence identity to a segment of amino acid sequence from a full-length wildtype ACE2. In one embodiment, the segment may start with an amino acid residue selected from the residue 1-17 of a full-length wildtype ACE2. In one embodiment, the segment may end with an amino acid residual selected from the residue 615-740 of the full-length wildtype ACE2. For example, the variant ACE2 domain may have an amino acid sequence having at least 98% or 99% sequence identity to a segment of amino acid sequence from residue 1 to residue 615, from residue 2 to residue 618, from residue 2 to residue 740, from residue 4 to residue 615, from residue 17 to residue 615, from residue 18 to residue 615, from residue 17 to residue 740, or any other combination of the starting residue and ending residue, from a full-length wildtype ACE2.

In one embodiment, the variant ACE2 domain comprises an amino acid sequence having at least 75%, 80%, 85%, 90%, 95%, 97%, 98%, or 99% sequence identity to SEQ ID NO. 3.

In one embodiment, the variant ACE2 domain may have a higher binding affinity to SARS-CoV, or SARS Spike protein than the full-length wildtype ACE2. For example, the variant ACE2 domain may have a binding affinity to SARS-CoV, or SARS spike protein with a KD from 0.1 nM to 100 nM.

In one embodiment, the variant ACE2 domain may have a higher binding avidity to SARS-CoV, or SARS Spike protein than the full-length wildtype ACE2. For example, the variant ACE2 domain may have a binding avidity to SARS-CoV, or SARS spike protein with a KD less than 10 nM.

In one embodiment, the fusion protein has avidity to Kappa variant less than 1.0E-12. In one embodiment, the fusion protein has a higher binding affinity to the delta SARS-CoV-2 strain than the Wuhan-Hu-1 strain. In one embodiment, the binding affinity to delta SARS-CoV-2 strain is at least 3 times that of the Wuhan-Hu-1 strain.

In one embodiment, the Fc domain is derived from a Fc domain of an immunoglobulin. The immunoglobulin may be IgG1, IgG2, IgG3, IgG4, IgA1 (d-IgA1, S-IgA1), IgA2, IgD, IgE, or IgM. In one embodiment, the Fc domain may have a Fc hinge region. In one embodiment, the Fc hinge region may be engineered to C220S. In one embodiment, the Fc domain may include a null mutation selected from K322A, L234A, and L235A when compared to a wildtype Fc domain. In one embodiment, the wildtype Fc domain has an amino acid sequence having at least 98%, or 99% sequence identity to SEQ ID NO. 5.

In one embodiment, the Fc domain may lack effector function. In one embodiment, the Fc domain may lack antibody-dependent cellular cytotoxicity (ADCC), antibody-dependent cellular phagocytosis (ADCP), and complement-dependent cytotoxicity (CDC). In one embodiment, the Fc domain comprises an IgG1 Fc domain.

In one embodiment, the Fc domain comprises an amino acid sequence having at least 75%, 80%, 85%, 90%, 95%, 97%, 98%, or 99% sequence identity to SEQ ID NO. 6.

In one embodiment, the fusion protein may have an amino acid sequence having at least 75%, 80%, 85%, 90%, 95%, 97%, 98%, or 99% of sequence identity to SEQ ID NO. 7, 9, 11, 13, 15, 16, 17, 18, 19, or 21.

In one embodiment, the fusion protein may have a molecular weight from about 50 kDa to 250 kDa. In one embodiment, the fusion protein may have a molecule weight of 50 kDa, 60 kDa, 70 kDa, 80 kDa, 90 kDa, 100 kDa, 120 kDa, 150 kDa, 180 kDa, 200 kDa, 250 kDa or any number in between.

In one embodiment, the fusion protein complex may be a homodimer of the fusion protein as disclosed herein. In one embodiment, the fusion protein complex includes two variant ACE2 domains. In one embodiment, the fusion protein complex comprises at least two fusion proteins. In one embodiment, the two fusion protein are paired through one or two disulfide bonds. In one embodiment, the disulfide bond is located on the hinge of the Fc domain.

In one embodiment, the fusion protein or fusion protein complex has a binding affinity to SARS-CoV-2, SARS-CoV, or SARS spike protein or a fragment thereof. In one embodiment, the binding affinity has an equilibrium dissociation constant (KD) not greater than 0.1 nM, 0.5 nM, 1 nM, 2 nM, 3 nM, 5 nM, 10 nM, 20 nM, 25 nM, 30 nM, 40 nM, 50 nM, 60 nM, 80 nM, or any number in between.

In one embodiment, the fusion protein or fusion protein complex has a binding avidity to SARS-CoV-2, SARS-CoV, or SARS spike protein or a fragment thereof. In one embodiment, the binding avidity has an equilibrium dissociation constant (KD) not greater than 1.0E-12, 0.001 nM, 0.01 nM, 0.05 nM, 1 nM, 2 nM, 3 nM, 5 nM, 10 nM, or any number in between.

In one embodiment, the fusion protein or fusion protein complex has a specific enzymatic activity from about from 50 pmol/min/µg to about 5000 pmol/min/µg. In one embodiment, the fusion protein has a specific enzymatic activity of about 568 pmol/min/µg.

The fusion protein is administrative in an effective dose for treating and preventing infections or diseases as disclosed herein. In one embodiment, the dose of the fusion protein administered per treatment is from about 1 mg/Kg to about 200 mg/Kg, from about 5 mg/Kg to about 100 mg/Kg, from about 3 mg/Kg to about 70 mg/Kg body weight, or from about 10 mg/Kg to about 150 mg/Kg.

In one embodiment, the dose of the fusion protein administered per day is less than or equal to about 100, 120, 140, 150, 180, 200 mg/Kg body weight. In one embodiment, the fusion protein is administered twice per day at a dose less than or equal to about 25, 50, 70, 90, 100, 150, 200 mg/Kg body weight.

In one embodiment, the fusion protein is administered as a liquid preparation. In one embodiment, the fusion protein is administered as a liquid suspension in a solution. In one embodiment, the solution may include comprising a salt, a carbohydrate, a surfactant, or a combination thereof. In one embodiment, the salt may be sodium chloride, histidine hydrochloride, or a combination thereof. In one embodiment, the carbohydrate may be a sucrose, glucose, or a combination thereof. In one embodiment, the surfactant may be a polysorbate 80.

In one embodiment, the liquid preparation may include the fusion protein in a concentration from about 2 mg/ml to about 20 mg/ml, from about 5 mg/ml to about 10 mg/ml, or from about 5 mg/ml to about 20 mg/ml.

The methods disclosed in this application may be used to treat or prevent a viral infection, acute respiratory distress syndrome, pulmonary arterial hypertension, or acute lung injury in a subject. In one embodiment, the administration of the fusion protein may prevent infection of the subject from the SARS-CoV-2 virus infection. In one embodiment, the administration of the fusion protein may reduce the risk of infection of the subject from the SARS-CoV-2 virus infection. In one embodiment, the administration of the fusion protein may prevent hospitalization of the subject having the SARS-CoV-2 virus infection. In one embodiment, the administration of the fusion protein may reduce the risk of hospitalization of the subject having the SARS-CoV-2 virus infection. In one embodiment, the administration of the fusion protein may reduce the length of hospital stay of the subject having the SARS-CoV-2 virus infection. In one embodiment, the administration of the fusion protein may prevent oxygenation and ventilation of the subject having the SARS-CoV-2 virus infection. In one embodiment, the administration of the fusion protein may reduce the needs for oxygenation and ventilation of the subject having the SARS-CoV-2 virus infection. In one embodiment, the administration of the fusion protein may prevent death of the subject having the SARS-CoV-2 virus infection. In one embodiment, the administration of the fusion protein may reduce the risk of death of the subject having the SARS-CoV-2 virus infection. In one embodiment, the administration of the fusion protein may reduce the severity of COVID symptom in the subject having the SARS-Co2-2 virus infection.

In one embodiment, the method may include administering the fusion protein or fusion protein complex intravenously, subcutaneously, through nasal passage (such as nasal spray), or through pulmonary passageway. In one embodiment, the fusion protein may be administered through daily infusion. In one embodiment, the fusion protein may be administered through daily intramuscular injections.

In one embodiment, the fusion protein may be co-administered with an antiviral agent, an immune regulatory reagent, or a combination thereof. In one embodiment, the antiviral agent may be favipiravir, ribavirin, galidesivir, remdesvir, or a combination thereof.

In one embodiment, the subject is a human. The methods disclosed in this application may be used on a subject having at least one of risk factor selected from the group consisting of an age greater than or equal to 65, a moderately or severely compromised immune system, a metabolic syndrome, being allergic to a COVID vaccine, and having low or no immune response after receiving a COVID vaccine. In one embodiment, the subject may have cancer, chronic kidney disease, chronic lung disease, diabetes, or heart disease.

In a further aspect, the application provides pharmaceutical compositions for treating disease or condition involving Angiotensin-Converting Enzyme 2 (ACE2). In one embodiment, the pharmaceutical composition includes the fusion protein or fusion complex as disclosed herein and a pharmaceutically acceptable carrier. In one embodiment, the pharmaceutical composition further includes an antiviral agent. In one embodiment, the pharmaceutical composition includes the protein-conjugate as disclosed thereof and a pharmaceutically acceptable carrier.

In one embodiment, the application provides a liquid composition, comprising the fusion protein as disclosed herein. In one embodiment, liquid composition comprises the fusion protein content from about 100 mg to about 20,000 mg, from about 200 mg to about 10,000 mg per dose, from about 100 mg to about 10,000 mg or from about 500 mg to about 10,000 mg.

In one embodiment, the liquid composition comprises the fusion protein in a concentration from about 0.1% to about 10%, about 0.5% to about 5%, about 0.5% to about 1% by weight, or about 0.5% to about 2%.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIGS. 1 shows (1A) the diagram of recombinant fusion proteins between ACE2 functional domain and engineered Fc (null) fragment (SI-69R2 and SI-69R4), (1B) the sequence of the SI-F019 fusion protein, a post-translational modified SI-69R2 devoid of N-terminal 17-amino acid signal peptide, (1C) the size-exclusion chromatograph indicating that the SI-F019 fusion protein complex is a homodimer, and (1D) the diagram of SI-F019-Spike protein complex;

FIGS. 2 shows that SI-F019, but not SI-69R4, is resistant to TMPRSS2-dependent hydrolysis (2A), and that the enzymatic activity of SI-F019 can be quantified in an in vitro fluorometric assay (2B);

FIG. 3 demonstrates that SI-F019 dose-dependent blockade of live SARS-CoV-2 infection to VeroE6 cells has reached 100% at all three MOI of virus in the test;

FIG. 4 shows that the addition of SI-F019 at 10 fM or above protected a portion of Vero E6 cells from undergoing cell lysis after 1-hour of viral infection by either SARS-CoV-2 or SARS-CoV-1 virus at a MOI of 0.01;

FIG. 5 shows that after preincubation with pseudovirus, SI-F019 inhibits viral infection in a dose-dependent fashion and achieves a complete inhibition at higher concentrations (IC50 = 32.56 nM);

FIGS. 6 shows the results of internalization/infection mediation assay that there was no uptake of GFP signals, indicative of pseudovirus (PsV), when pretreated with SI-F019 in the concentrations tested, while low GFP signals were associated with SI-69C1 (anti-S1 antibody) and SI-69R3 (SARS-CoV-2 ACE-2 Fc WT), as well as media, buffer, and ACE2-his (SI-69C1), at 48 hours in THP1 (pH 7.2)(6A), THP1 (pH 6.0)(6B), and Daudi (6C);

FIG. 7 shows that SI-F019 can compete against either a natural anti-SARS-CoV-2 antibody or an ACE2-Fc (wild type) fusion protein to block the Fc mediated antibody-dependent enhancement (ADE) as measured by GFP signals, indicative of PsV infection;

FIG. 8 shows the flow cytometry analysis of the HEK293-T cells expressing SARS-CoV-2 Spike protein as detected by using anti-Spike antibody and anti-human Fc antibody;

FIG. 9 shows the dose-dependent binding of SI-F019 to the HEK293-T cells expressing SARS-CoV-2 Spike protein as measured by geometric mean fluorescent intensity (gMFI);

FIG. 10 displays the FACS analysis of antibody-dependent cellular cytotoxicity (ADCC) assay showing that a human anti-S1 antibody (SI-69C3) directs human NK cells to target the HEK293-T cells expressing SARS-CoV-2 Spike protein, as measured by Calcein-AM and Propidium Iodide staining;

FIGS. 11 shows that when compared to a human anti-S1 antibody (SI-69C3), the dose-binding response of SI-F019 and control molecules on HEK293-T cells in an ADCC assay (11A), and that SI-F019 did not mediate ADCC at the treatment doses between 100fM and 100 nM, whereas its variant with wild type Fc (SI-69R3) did in a dose-dependent fashion, even though the level of activity was lower (11B);

FIG. 12 shows that the Fc null mutations enable SI-F019 to reduce the serum-mediated complement-dependent cytotoxicity (CDC) in vitro as measured by the viability of HEK293-T cells expressing SARS-CoV-2 S protein;

FIGS. 13 shows that SI-F019 does not induce serum complement-dependent cytotoxicity (CDC) in vitro by measuring the viability of HEK293-T cells expressing SARS-CoV-2 S protein after various treatments (13A); and the Fc null mutations of SI-F019 have no effect on the subsequent cell growth at 96 hours post treatment in vitro (13B);

FIGS. 14 shows that SI-F019 does not elicit the release of cytokines in PBMC culture in either soluble or plate-bound form: (14A) IFNγ; (14B) TNFα; (14C) GM-CSF; (14D) IL-2; (14E) IL-10; (14F) IL-6; (14G) IL-1β; (14H) IL-12p70; and (14I) MCP-1.

FIGS. 15 shows the biolayer interferometry of binding kinetics (affinity) of SI-F019 (15a) and neutralizing antibodies to COVID-19 RBD variants, including Bamlanivimab (SI-69C4)(15b), Casirivimab (SI-69C5)(15c), Etesevimab (SI-69C6)(15d), Imdevimab (SI-69C7)(15e), Cilgavimab (SI-69C8)(15f), and Tixagevimab (SI-69C9)(15 g);

FIGS. 16 shows the biolayer interferometry of binding kinetics (avidity) of SI-F019 (16a) and neutralizing antibodies to RBD variants including Bamlanivimab (SI-69C4)(16b), Casirivimab (SI-69C5)(16c), Etesevimab (SI-69C6)(16d), Imdevimab (SI-69C7)(16e), Cilgavimab (SI-69C8)(16f), and Tixagevimab (SI-69C9)(16 g); and

FIGS. 17 demonstrates the potency of SI-F019 in protecting ACE2-expressing 293T cells from viral inhibition using variants of S protein packaged pseudovirus (NICPBP) in a luciferase reporter assay (17a), and the linear correlation between IC50 and the binding affinity (17b) or avidity (17c) indicative of a competitive inhibition by SI-F019.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present application relates to, among others, the generation and characterization of fusion proteins such as recombinant human ACE2-Fc fusion proteins. In some embodiments, these fusion proteins are capable of protecting the membranous ACE2 of human host cells from the viral particles or virus. In one embodiment, the viral particles or virus may utilize viral spike proteins for viral entry into host cells after infection. In one embodiment, the viral particles include, but not limited to, SARS-CoV-2 virus, COVID-19 virus, variants of SARS-CoV-2, and other coronaviruses. In one embodiment, the virus may cause severe acute respiratory syndrome (SARS). In one embodiment, the SARS may include coronavirus disease 2019 or COVID-19.

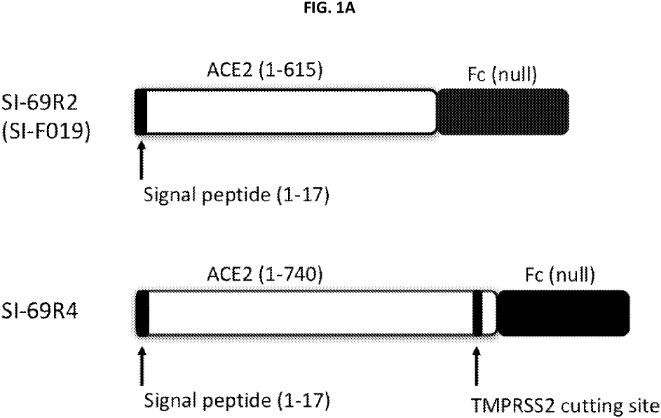

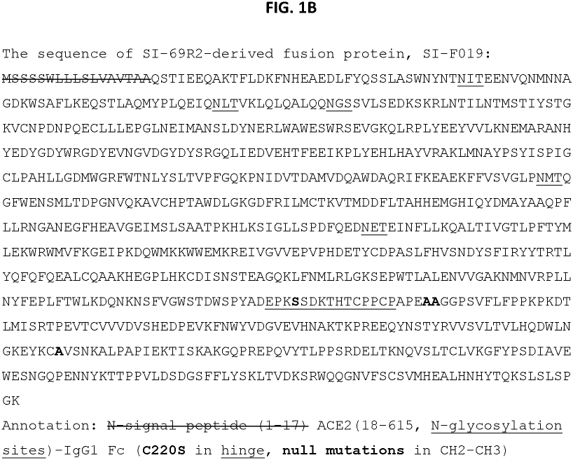

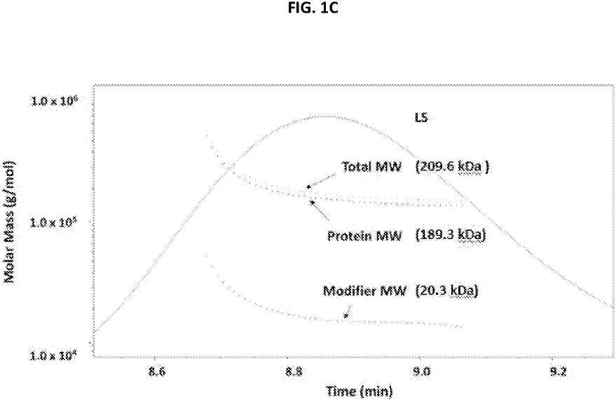

In one embodiment, the recombinant human ACE2-Fc fusion proteins may be a fusion protein of ACE2 zinc metallopeptidase domain (also known as ACE2 extracellular domain, ACE2-ECD) and IgG1 Fc fragment. In one embodiment, the fusion protein is SI-F019, a fusion protein of ACE2-ECD and IgG1 Fc fragment with mutations of C220S, L234A, L235A, and K322A according to EU numbering system (Table 1 and FIGS. 1). An active ACE2-ECD retains the structural conformation for the host receptor-virus interaction. Each mutation in IgG1 Fc fragment may deplete certain immune responses. The mutation C220S may remove unpaired cysteine for pairing heavy and light chains and thus providing the technical advantage of, among others, avoiding protein forming aggregate, improving protein stability and promoting manufacture efficiency and scalability. The introduction of both L234A and L235A may reduce the effector function of Fc, such as antibody-dependent cellular cytotoxicity (ADCC), antibody-dependent cellular phagocytosis (ADCP). The K322A mutation may reduce C1q binding triggered complement-dependent cytotoxicity (CDC). SI-F019 is designed to neutralize SARS-CoV-2 virus while trigging fewer effector response.

The terms “a”, “an” and “the” as used herein are defined to mean “one or more” and include the plural unless the context is inappropriate.

The term “recombinant fusion protein” refers to a protein that is created through genetic engineering of a fusion gene encoding two or more genes that originally coded for separate proteins.

The term “ACE2-Fc” refers to a recombinant fusion protein of a human ACE2 protein fragment and an engineered fragment of the fragment crystallizable region (Fc region) of a human immunoglobulin, where the human Immunoglobulin including, but not limited to, IgG1, IgG2, IgG3, IgG4, IgA1 (d-IgA1, S-IgA1), IgA2, IgD, IgE, and IgM.

The term “spike”, “Spikes”, “S protein”, or variants refers to the protein responsible for allowing the virus to attach (“S1 subunit” or “S1 protein”) to and fuse (“S2 subunit” or “S2 protein”) with the membrane of a host cell. In the case of COVID-19, SARS-CoV-2 has sufficient affinity to the ACE2 receptor on human cells to use them as a mechanism of cell entry, and SARS-CoV-2 has a higher affinity to human ACE2 than the original SARS virus.

The term “Fc domain”, “Fc fragment”, and “Fc region” refer to the identical domain or fragment of the Fc region (“Fc domain” and “Fc fragment”, respectively) in IgG, IgA, and IgD antibody isotypes, which is derived from the hinge, and the second and third constant domains (CH2-CH3) of the antibody’s two heavy chains.

The term “affinity” refers to a measure of the attraction between two polypeptides, such as receptor/ligand, ACE2/spike protein or it’s variants, for example. The intrinsic attractiveness between two polypeptides can be expressed as the binding affinity equilibrium constant (KD) of a particular interaction. A KD binding affinity constant can be measured, e.g., by Bio-Layer Interferometry.

The term “avidity” refers to the accumulated strength of multiple affinities of individual non-covalent binding interactions, such as between a protein receptor and its ligand, and is commonly referred to as functional affinity. As such, avidity is distinct from affinity, which describes the strength of a single interaction.

The term “antigenic drift” refers to random genetic mutation of an infectious virus resulting in a new strain of virus with minor changes in antigenicity, to which the antibodies that prevented infection by previous strains may not be effective.

The term “cytokine release syndrome” (CRS) refers to CRS in severe cases of COVID-19 associated with an increased level of inflammatory mediators including cytokines and chemokines, such as interleukin (IL)-2, IL-6, IL-7, IL-10, tumor necrosis factor (TNF), granulocyte colony-stimulating factor (G-CSF), monocyte chemoattractant protein-1 (MCP1; also known as CCL2), macrophage inflammatory protein 1 alpha (MIP1α; also known as CCL3), CXC-chemokine ligand 10 (CXCL10), C-reactive protein, ferritin, and D-dimers in blood upon SARS-CoV-2 infection.

The term “neutralizing antibody” refers to an antibody that defends a cell from a pathogen or infectious particle by neutralizing any effect it has biologically. Neutralization renders the particle no longer infectious or pathogenic. Neutralizing antibodies are part of the humoral response of the adoptive immune system against viruses, intracellular bacteria, and microbial toxin. By binding specifically to surface structures (antigen) on an infectious particle, neutralizing antibodies prevent the particle from interacting with its host cells it might infect and destroy. Immunity due to neutralizing antibodies is also known as sterilizing immunity, as the immune system eliminates the infectious particle before any infection takes place.

The term “vaccine” refers to a biological preparation that provides active acquired immunity to a particular infectious disease. Vaccines can be prophylactic (to prevent or ameliorate the effects of a future infection by a natural or “wild” pathogen), or therapeutic (to fight a disease that has already occurred).

The term “breakthrough infection” refers to a case of illness in which a vaccinated individual becomes sick from the same illness that the vaccine is meant to prevent. The character of breakthrough infections is dependent on the virus itself. The infection in the vaccinated individual often results in milder symptoms and is of a shorter duration than if the infection was contracted naturally. The causes of breakthrough infections include age, mutations in viruses and neutralizing antibodies, improper administration or storage of vaccines.

The term “sterilizing immunity” refers to immunity due to neutralizing antibodies capable of inhibiting the infectivity by binding to the pathogen (e.g. all SARS-CoV-2 variants) and blocking the molecules (i.e. Spike coded by variants) needed for cell entry, with which infection is prevented completely. Because of the breakthrough infections, none of COVID-19 vaccines nor neutralizing antibodies offer full sterilizing immunity. By these definitions, SI-F019 may be used as a therapeutic vaccine to achieve therapeutic sterilizing immunity to variants of SARS-CoV-2 viruses, as well as any other SARS viruses that use ACE2 as viral entry into human cells.

### EXAMPLES

### Example 1. The Cloning, Expression, and Purification of Recombinant ACE2-Fc Fusion Proteins

Human membranous ACE-2 is the receptor critical for mediating SARS-CoV viral entry into host cells in human. The human ACE2 protein has at least three functional domains: a signal peptide (residues 1-17), zinc metallopeptidase domain (residues 18-615), and a TMPRSS2 protease cutting site (residues 697-716) (SEQ ID NO. 1 is the full length human ACE2 protein sequence from Genbank number: NP_001358344.1), of which the SARS-CoV viral protein, Spike, interacts with the zinc metallopeptidase domain (SEQ ID NO. 3 is the protein sequence of truncated ACE2 from residue 1 to 615). On the other hand, the Fc region of a human antibody (SEQ ID NO. 5) is capable of interacting with Fc receptors (FcRs) on many immune cells and some proteins of the complement system. Each Fc fragment of IgG1 Fc region contains a cysteine at C220 (according to EU numbering system), which may intrinsically form disulfide bond with either kappa or lambda light chain. To reduce the risk of having a free cysteine that may destabilize and/or inactivate the protein, C220 may be substituted for serine (C220S) or other amino acids. To reduce the Fc binding to FcγR and C1q, other point mutations, such as K322A, L234A, and L235A, may be engineered into wild type IgG1 Fc fragment. Collectively, the IgG1 Fc fragment harboring the four mutations is called IgG1 Fc null (SEQ ID NO. 6).

The recombinant human ACE2-Fc fusion proteins (as listed in Table 1) were engineered to produce soluble fusion proteins, of which SI-69R2 (SEQ ID NO. 7) is a recombinant fusion protein of a truncated ACE2 fragment without the TMPRSS2 protease cutting site and the IgG1 Fc null fragment. Other recombinant fusion proteins were created to provide a Fc fragment of Ig isotype, such as SI-69R2-G4 (IgG4 Fc, SEQ ID NO. 9), SI-69R2-A1 (IgA1 Fc, SEQ ID NO. 11), SI-69R2-A2(IgA2 Fc, SEQ ID NO. 13), or wild type IgG1 Fc fragment (IgG1 Fc, SEQ ID NO. 19). The recombinant fusion protein of a truncated ACE2 with all three domains and a wild type IgG1 Fc fragment was also created (SI-69R4, 1-740, SEQ ID NO. 21). Of all recombinant ACE2-Fc fusion proteins, the signal peptide (ACE2 residues 1-17) may be replaced with other signal peptides at different lengths, without affecting the function of other domains in either human ACE protein or ACE2-Fc fusion proteins.

The recombinant fusion genes encoding the fusion proteins in Table 1 were cloned into either pCGS3.0 (such as SI-69R2) or pTT5 expression vector (such as SI-69R4 and SI-69R10) and expressed in ExpiCHO cells. All the fusion proteins were purified following standard protein expression protocols, sterilized using a 0.22 um filter, and stored in a cryopreservation buffer at 4° C. During the expression and purification, each recombinant fusion protein may undergo post-translational modification, including N-glycosylation and the cleavage of N-terminal signal peptide (17 amino acids). In case of SI-69R2, the purified fusion protein was given a new name, SI-F019.

As shown in FIGS. 1A and 1B, SI-F019 retains the truncated ACE2 fragment (residues 18-615) encompassing the zinc metallopeptidase domain (residues 19-611) of human ACE2 but not the TMPRSS2 protease cutting site. In addition, SI-F019 retains the IgG1 Fc null fragment devoid of its binding to Fcy receptors. In this way, SI-F019 in its soluble form is not expected to bind any target cells in peripheral blood.

The SI-F019 fusion protein likely undergoes post-translation modification, such as N-glycosylation, and homodimerization linked by the two disulfide bonds of Fc region. To assess the actual molecule weight of the SI-F019 dimer, the analytical size exclusion chromatography (SEC) was used, in a combination of multi-angle light scattering (MALS), absorbance (UV), and/or refractive index (RI) concentration detectors techniques, as shown in FIG. 1C. The method combines the chromatographic separation by molecular size and the determination of absolute molar mass by light scattering (LS) without the limitations of molecule weight standard calibration. SI-F019 exhibited an average total molecular weight of 209.6 kDa (main peak), of which the molecule weights of the SI-F019 dimer and its modifiers (i.e. glycans) were measured at 189.3 kDa and 20.3 kDa, respectively. In the theoretical calculation of its amino acids, the molecule weight of the SI-F019 monomer is 95.1 kDa. Thus, the purified SI-F019 fusion protein complex is a homodimer, whereas SI-F019 protein complex refers to the protein-protein interaction between SI-F019, as either a monomer or a dimer, and other proteins, such as spike proteins and effector proteins. The formation of SI-F019-Spike protein complex (as illustrated in FIG. 1D) underlies the mechanism by which SI-F019 is a candidate inhibitor for preventing SARS-CoV-2 virus from docking onto the membranous ACE2 for viral entry into the host cells in human.

### Example 2: The Binding of SI-F019 to Spikes, Fc Receptors, and C1q

SI-F019 was designed to block SARS-CoV viral entry into human by preventing the spike proteins from binding to the membranous ACE2 protein on human host cells. Spikes are the most distinguishing feature of coronaviruses, which are the knob-like structures responsible for the corona- or halo-like surface. The spike proteins are generally composed of glycoproteins, and each spike is composed of a trimer of the Spike protein, and the S protein is in turn composed of an S1 and S2 subunit. The homotrimeric Spike protein mediates the receptor binding and membrane fusion between the virus and host cell. The S1 subunit forms the head of the spike and has the receptor-binding domain (RBD). The S2 subunit forms the stem which anchors the spike in the viral envelope and on protease activation enables fusion. In a functionally active state, the subunit complex of S1 and S2 is split into individual subunits when the virus binds and fuses with the host cell under the action of proteases, such as cathepsin family and transmembrane protease serine 2 (TMPRSS2) of the host cell. Spikes play important roles in the viral entry of infection process by coronavirus. In case of COVID-19, SARS-CoV-2 virus docks onto the membrane bound ACE2 receptor on the host cell surface, and the interaction between spikes and the functional domain of ACE2 brings about the release of viral nucleocapsid into the host cell cytoplasm by triggering fusion between the viral envelope and host cell membranes.

SI-F019 was evaluated for the binding affinity and avidity of ACE-Fc fusion proteins to the viral spike proteins. In a Bio-Layer Interferometry analysis, the samples of spike proteins include SARS-CoV-2 spike trimer, SARS-CoV-2 S1 protein, SARS-CoV-2 S1 protein RBD domain, and SARS-CoV-1 RBD domain (Table 2). These reagents were purchased from ACROBiosystems. The binding affinity assay measured the binding of SI-F019 immobilized on the anti-human IgG Fc Capture Biosensors tip (AHC) surface to the spike protein or it’s subunit in solution. The avidity assay measured the binding of a biotinylated spike protein immobilized on the Streptavidin Biosensors tip (SA) surface to SI-F019 in solution. These reagents were chemically biotinylated by NHS-ester activated reaction, with the stoichiometric ratio of biotin/protein is 2:1. The data analysis utilized a 1:1 fitting model to calculate both the binding affinity and avidity. The result indicates that the binding affinity and avidity of SI-F019 to these spike proteins, fragments, or domains seem to be within their respective scales of KD in nanomolar (nM) (Table 2). This characteristic and informative data may be useful references for measuring the SI-F019 protein complex with variants of viral spike proteins indicative of potential antigenic drift among SARS-CoV-2 variants. Indeed, this type of viral mutations has been identified in certain strains of SARS-CoV-2 virus, such as D614G in the spike protein (Zhang et al., 2020) that altered the viral affinity to membranous ACE2 and viral entry into the host cells.

In parallel to its binding to spikes, SI-F019 was evaluated for its binding to human FcγRs, C1q, and FcRn by using Bio-Layer Interferometry. As shown in Table 3, the binding to FcγRs, including FcγRI, FcγRIIa, FcγRIIb, and FcγRIIIa, was not detected, nor the binding to C1q. However, SI-F019 did bind to FcRn and the binding affinity was determined at a KD of 37.6 nM, which is comparable to that of human IgG1 Fc region.

### Example 3. SI-F019 is Resistant to TMPRSS2 Protease Activity

Human ACE2 is subject to membranous protease hydrolysis by TMPRSS2, and monomeric extracellular ACE2 is shed from cells, which can be readily detected in serum. In the recombinant ACE2-Fc fusion proteins, the truncated ACE2 domain is fused to Fc fragment but still retains the binding affinity to the viral spike proteins.

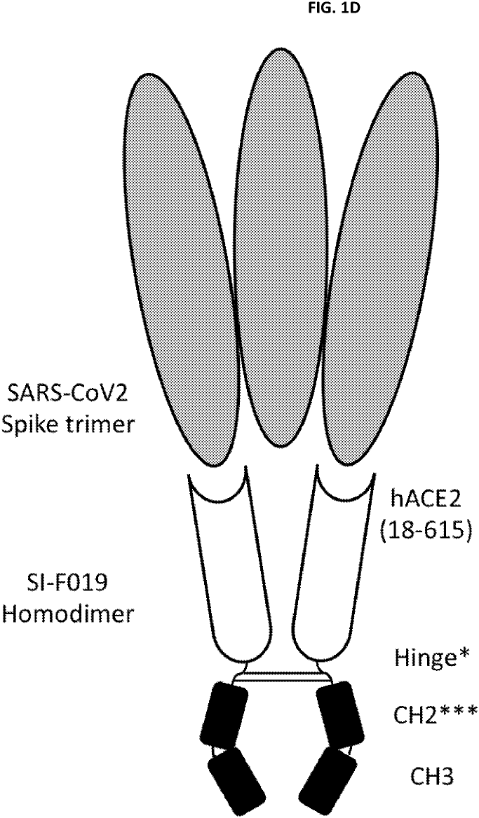

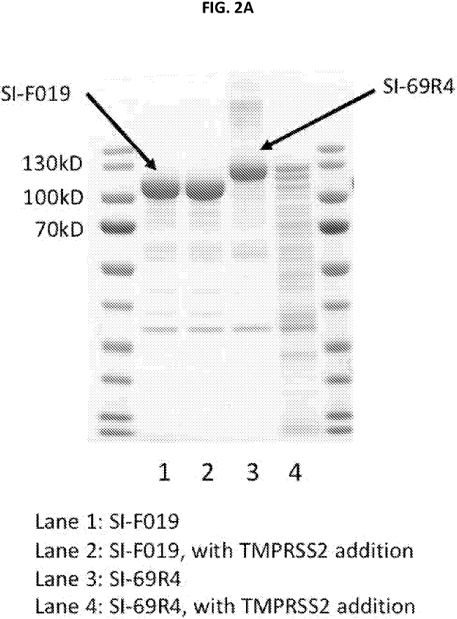

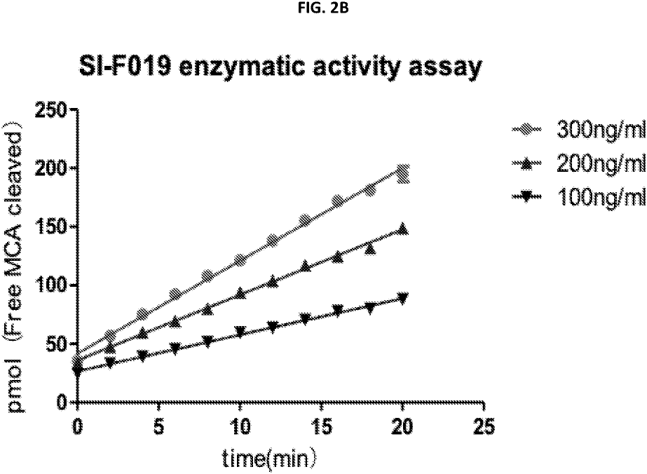

SI-F019 was engineered without the TMPRSS2 cutting site in the truncated ACE2 domain. As shown in FIGS. 1, SI-F019 contains residues from 18 to 615, whereas SI-69R4 encodes all three ACE2 domains (residue: 1-740, SEQ ID NO. 21) encompassing the TMPRSS2 cutting site. To demonstrate that SI-F019 is free from TMPRSS2-specific proteolysis, SI-69R4 was used as a control. To carry out the assay of TMPRSS2-specific hydrolysis, the TMPRSS2 (106-492) catalytic domain was cloned, expressed, and purified according to Genbank: NP_001358344.1. As shown in FIG. 2A, in the absence of TMPRSS2, both SI-F019 and SI-69R4 stably migrated to their respective molecule weights (as monomers under reducing, denaturing condition). When TMPRSS2 was added, SI-F019 revealed its resistance to TMPRSS2, whereas SI-69R4 underwent proteolysis indicating its sensitivity to TMPRSS2 as predicted. Thus, SI-F019 is stable and is resistant to TMPRSS2-mediated protease activity.

### Example 4. SI-F019 Exerts the Enzymatic Activity of ACE2

SI-F019 is a fusion protein of a truncated ACE2 (residue 18-615) and IgG1 Fc null fragment. The truncated ACE2 encodes a zinc metallopeptidase, whose enzymatic activity may be reevaluated by using an established assay. A peptide substrate of ACE2 with an MCA (7-Methoxycoumarin-4-acetic acid) fluorescent tag [MCA-YVADAPK (Dnp)-OH_Fluorogenic Peptide Substrate] was used to measure ACE2 enzymatic activity of SI-F019. MCA molecule was prepared as standard curve calibration for free fluorophore quantification, and the substrate was diluted in DMSO to 0.97 mg/ml. SI-F019 was diluted to 100, 200, and 300 ng/ml and used to cleave fluorogenic peptide in-vitro to release free MCA. The assay was incubated at room temperature for 20 minutes, and data were collected for fluorescent signals at timepoints with 2 minute intervals.

The cleaved MCA was quantified in molar using MCA standard curve. The enzymatic activity was determined according to the slope of linear curve as shown in FIG. 2B (MCA quantity against time). SI-F019 showed good linearity (R²> 0.99) at all three concentrations, indicating that the stable cleavage of peptide was concentration-dependent. For calculating the enzymatic activity, the slope was divided by mass number (µg) of SI-F019. The final specific enzymatic activity was 568 pmol/min/µg. The fact that SI-F019 retains the enzymatic activity of the membranous ACE2 indicates that this independent domain of ACE2 also retains the structural conformation for the host receptor-virus interaction.

### Example 5 SI-F019 Inhibits Live SARS-CoV-2 Infection to VeroE6 Cells

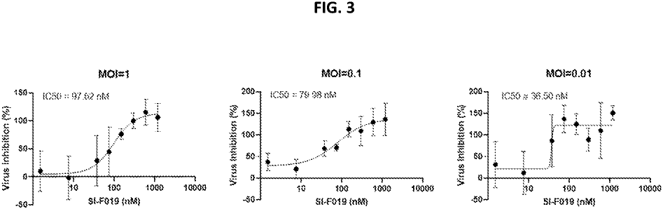

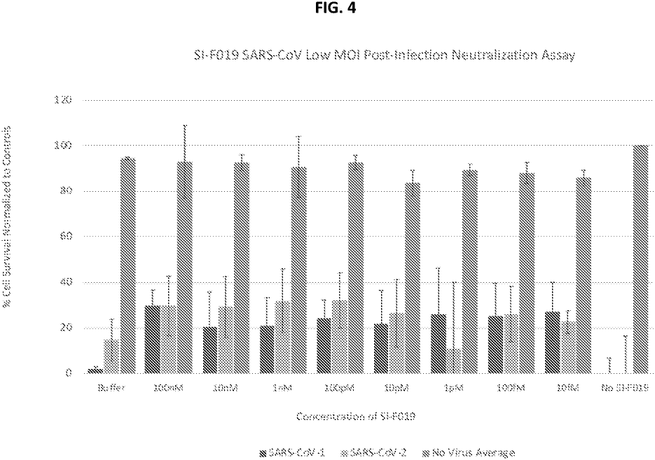

SI-F019 was tested for the ability to inhibit live SARS-CoV-2 infection and lysis of VeroE6 (ATCC: CRL-1586) cells in vitro. SI-F019 test concentrations, ranging from 1.5 nM to 1200 nM, were preincubated with 3 concentrations of live SARS-CoV-2 virus (Strain USA-WA1/2020, representing a 100-fold range of Multiplicity Of Infection, MOI) for 1 hour and then added to 90% confluent monolayer of VeroE6 cells. After 1 hour, the medium containing the virus was removed and replaced with the medium containing SI-F019 at matching test concentrations, and the tests were conducted in triplicate. The cell viability was measured by neutral red dye uptake after 72 hours and the percentage of inhibition of lytic viral infection was determined by comparison to wells in which virus was added at each MOI without SI-F019. The 50% inhibitory concentrations (IC50) for each virus concentration (1 MOI = 40,000 virus particles) were calculated using GraphPad Prism software and are shown on each graph. The preincubation of SI-F019 with live SARS-CoV-2 resulted in a dose-dependent blockade of infection that reached 100% at all three MOI of virus that were tested. As shown in FIG. 3, SI-F019 neutralized as much as 40,000 virus particles at a MOI of 1.0, with an IC50 of 97.62 nM. At MOI of 0.1 and 0.01, SI-F019 was able to block the infection at IC50 of 79.95 nM and 36.5 nM, respectively.

### Example 6. SI-F019 Reduces Virus Replication and Reinfection

SI-F019 was tested for its ability to inhibit replication and reinfection, i.e. further transfer of infection to VeroE6 cells from the cells previously infected with a low MOI of SARS-CoV-2 or SARS-CoV-1 viruses. VeroE6 cells in a 90% confluent monolayer (~20,000 cells) were exposed to either SARS-CoV-2 (Strain USA-WA1/2020) or SARS-CoV-1 (Strain Urbani 2003000592) for 1 hour at a MOI of 0.01 (calculated as 400 virus infective particles). After washing out free virus particles, SI-F019 was added to the cells in a range from 10 fM to 100 nM in triplicates and the cell culture was maintained for 72 hours. Cell viability was determined by neutral red dye uptake and % inhibition of viral cytotoxicity was calculated. Absorbance values were normalized on each plate using the maximum absorbance of the conditions with no virus or no drug (NVND) representing 100% cell viability, and the average absorbance value of the virus/no drug (VND) establishing the maximum cell death using the formula:

$$\begin{matrix}\text{\% Cell Survival} = \left[\left({\text{Well OD}}_{540} {\text{-VND OD}}_{540}\right) / \left({\text{NVND OD}}_{540} \text{-}\right)\right) \\ \left(\left({\text{VND OD}}_{540}\right)\right] * 100\end{matrix}$$

As shown in FIG. 4, the addition of SI-F019, at a concentration of 10 fM protected Vero E6 cells from secondary infection. The culture infected with either SARS-CoV-2 or SARS-CoV-1 virus at an MOI of 0.01 for 1-hour reduced the cell lysis by at least 20%. However, no significant increase in protection was observed in this assay when the concentration of SI-F019 was increased by 10-fold increments up to 100 nM. The finding indicates that, to the cells infected with a low titer of virus, the addition of SI-F019 may reduce the spread of virus as well as the degree of cytotoxicity, even at low concentrations.

### Example 7. SI-F019 Inhibits Pseudo-Virus Infection of HEK293T-ACE2 Cells

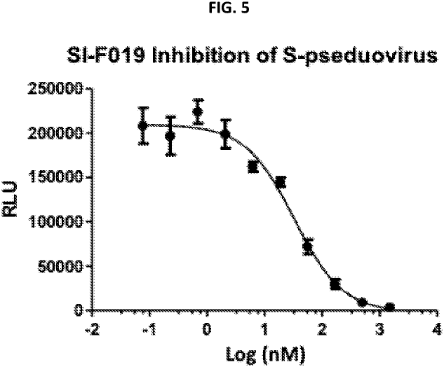

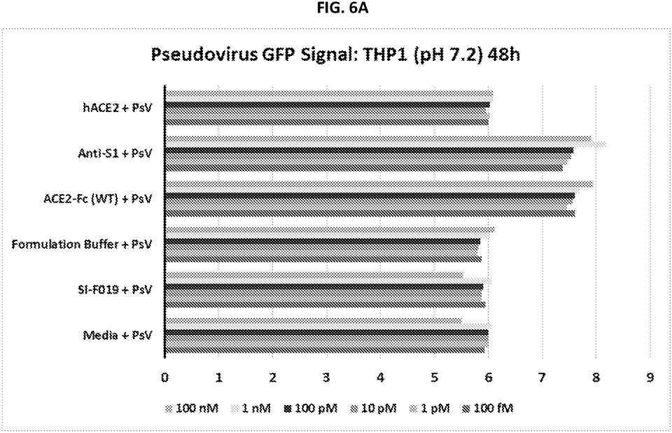

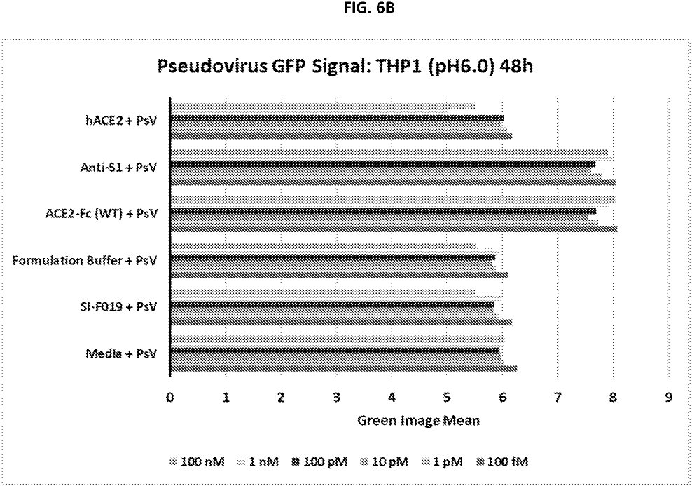

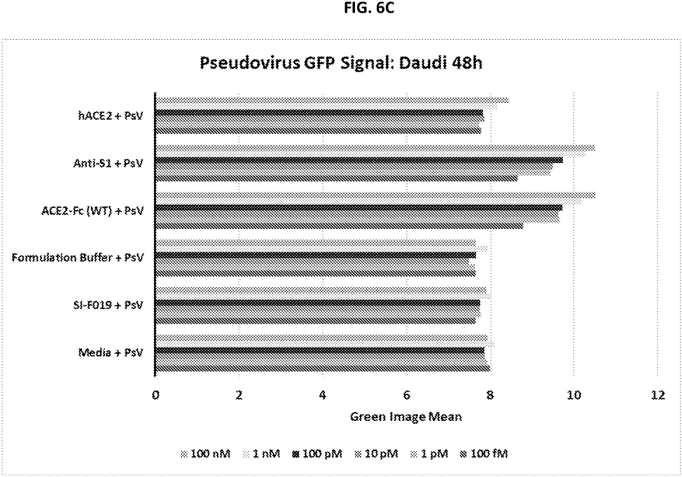

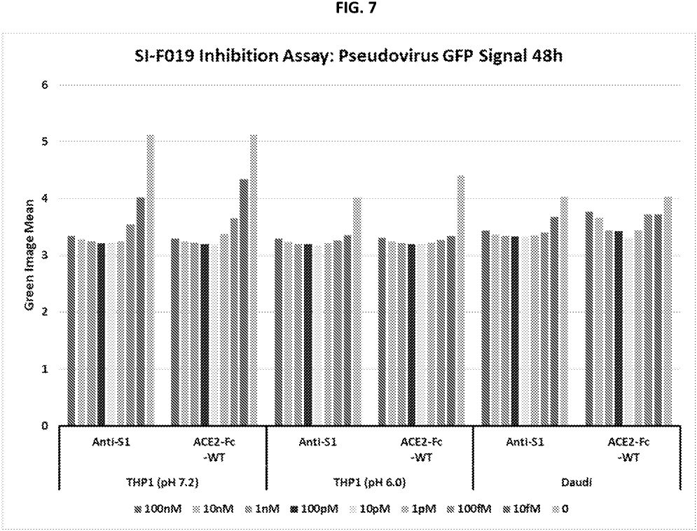

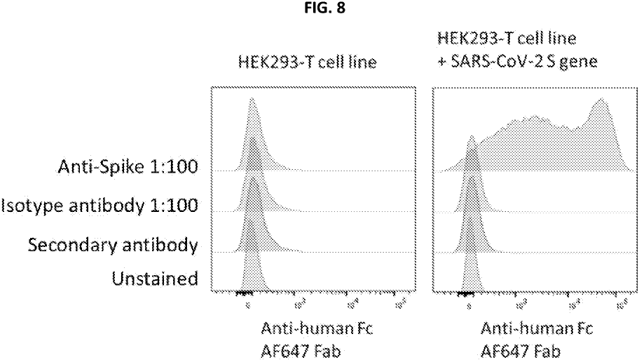

HEK293T (ATCC: CRL-3216)-3D4 clone cell line was generated by lentiviral transduction of human ACE2 protein. The function of expressed human ACE2 was confirmed by enzymatic substrate conversion assay and binding by specific antibody by FACS. SARS-CoV-2 S protein packaged pseudo-virus which containing a luciferase reporter gene was obtained from National Institute for the Control of Pharmaceutical & Biological Products. Testing was conducted according to the manufacturer’s instructions. The S-pseudo virus stock solution was diluted in culture medium with MRD of 20 in order to yield 300 TCID50/well of virus load. SI-F019 at concentrations ranging from 0.07 nM to 1500 nM were preincubated with the diluted virus solution for 1 hour. HEK293T-3D4 cells were dispersed into a 96-well plate. After 1 hour, mixtures were added into cell plate. Infected cells were measured by testing luciferase activity after 24 hours of incubation. 50% inhibitory concentrations (IC50) for defined virus load were calculated using GraphPad Prism software. FIG. 5 shows that after preincubation with pseudo-virus, SI-F019 inhibits viral infection in a dose-dependent fashion and achieves a complete inhibition at higher concentrations (IC50= 32.56 nM).

### Example 8. SI-F019 Reduces the Incidence of ADE

Antibody-dependent enhancement (ADE) is a phenomenon in which binding of a virus to suboptimal antibodies enhances its entry into host cells. In case of COVID-19, the secondary infection of SARS-CoV-2 virus to the patient who has anti-SARS-CoV-2 antibodies developed from a primary infection or to an individual who has been vaccinated may lead to enhanced uptake of virus by monocytes and B cells. The anti-virus antibodies in contact with the virus may bind to Fc receptors expressed on certain immune cells or some of the complement proteins. The latter binding depends on the Fc region of the antibody. Typically, the virus undergoes degradation in a process called phagocytosis, by which viral particles are engulfed by host cells through plasma membrane. However, the antibody binding might result in virus escape if the virus is not neutralized by an antibody, either due to low affinity binding or targeting a non-neutralizing epitope. Then, the outcome is an antibody enhanced infection.

The antibodies developed through either natural immunity or vaccination possess a wild type Fc region. While SI-F019 is capable of competing with anti-spike antibodies for binding to SARS-CoV2 virus, the IgG1 Fc null fragment is incapable of binding to either Fc receptors or C1q (see Table 3). To demonstrate its comparative advantage in reducing the effect of ADE, SI-F019 was evaluated for its role in internalization, replication, and reinfection.

In an assay for measuring Fc mediated internalization, the SARS-CoV-2 S protein was packaged into GFP-expressing pseudo-virus (PsV), and two cell lines, THP1 (monocyte) and Daudi (B cell) that express Fc receptors and complement receptor 2 (CR2), were used for testing FcRg and CR2-mediated ADE mechanisms. SI-69R3 was used as a control for SI-F019, having a wild type Fc in contrast to SI-F019 that has an IgG1 Fc null modification (see Table 1). After being exposed to PsV for 48 hours, the green fluorescent signal from the cells was quantified as an indicator of PsV infection. In the conditions treated with PsV and SI-69C1, anti-S1 antibody, or SI-69R3 low levels of green fluorescence were measured at 48 h in THP1 (pH 7.2) (6A), THP1 (pH 6.0)(6B), and Daudi (6C) cells. This result indicated that some transfer of PsV could occur via the Fc receptor. In contrast, the condition with SI-F019 at the indicated concentrations resulted in no uptake of PsV by THP1 or Daudi cells, comparable with the green fluorescent signal measured in the negative control conditions including, assay media, formulation buffer, and SI-69C1(FIGS. 6). The effect of Fc mediated ADE was dose-dependent, as the treatment to the cells was carried out using doses ranging from 1 pM up to 100 nM. This indicates that some uptake of PsV may occur through either FcγR or CR2 mechanisms.

### Example 9. SI-F019 Reduces Virus Load of PsV

SI-F019 may not mediate the internalization of S protein packaged GFP-expressing pseudo-virus (PsV) due to lack of a functional Fc fragment. To determine if SI-F019 can inhibit the uptake of the pseudovirus, SI-F019 was used as a co-treatment with either SI-69R3 or natural anti-SARS-CoV-2 antibody in a competition mode. The PsV was incubated for 1 hour with SI-F019 at a dose range from 1 pM to 100 nM, together with either 10 pM of anti-SARS-CoV-2 (S1) antibody or 10 pM of SI-69R3 prior to infecting the same set of target cells. PsV derived GFP signals were detected as the virus load of infection. SI-F019 was able to inhibit the virus load of PsV in the target cells starting at 10 fM (FIG. 7).

While both the antibody, such as anti-SARS-CoV-2 (S1) antibody, and the fusion protein of truncated ACE2-wild type Fc fragment in SI-69R3, were shown to be able to mediate internalization of SARS-CoV-2 Spike pseudotyped lentivirus, SI-F019 failed to do so due to lack of a functional Fc fragment. Herein, SI-F019 helped reduce virus load of PsV in the presence of either 10 pM of anti-SARS-CoV-2 (S1) antibody or 10 pM of SI-69R3, even at a low concentration of 10 fM. Together, these results indicate that SI-F019 may reduce the incidence of ADE induced by FcRg and CR2 dependent mechanisms in THP1 monocytes and Daudi B cells, respectively.

### Example 10. HEK293-T Cells Expressing SARS-CoV-2 Spike Protein

HEK293-T cells (ATCC: CRL-3216) that stably express SARS-CoV-2 spike protein were established by transducing the lentivirus packaged with SARS-CoV-2 spike protein encoding cDNA (Accession: YP_009724390.1) and IRES expression and selection based on puromycin resistance driven by same expression construct (LPP-CoV219-Lv105-050, GeneCopoeia). The expression of SARS-CoV-2 spike protein was confirmed by binding of a human IgG clone AM001414, specific for SARS-CoV-2 Spike protein “Anti-Spike”, (SKU938701, Biolegend) and the Human IgG Isotype matched clone QA16A12 was used as control “Isotype”, (SKU403502, Biolegend). Bound protein was quantified by secondary incubation with polyclonal anti-human Fc AF647 Fab (SKU109-607-008, Jackson ImmunoResearch) and FACS evaluation as shown in FIG. 8.

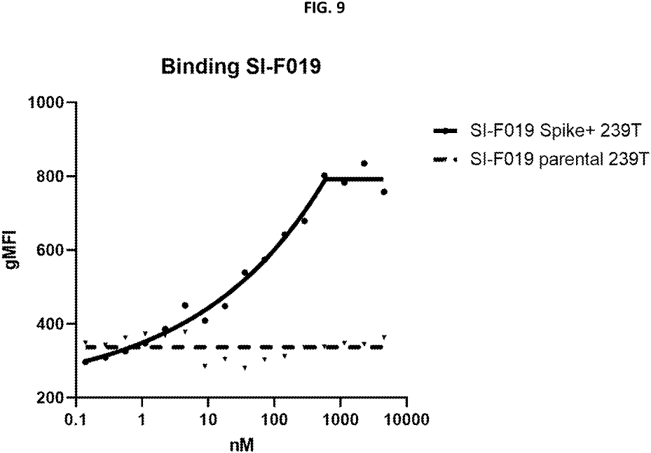

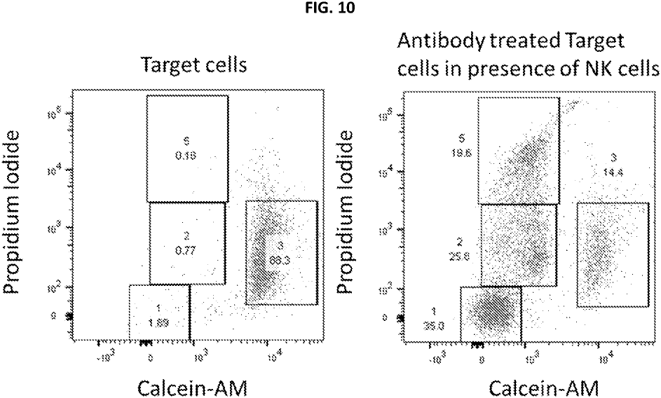

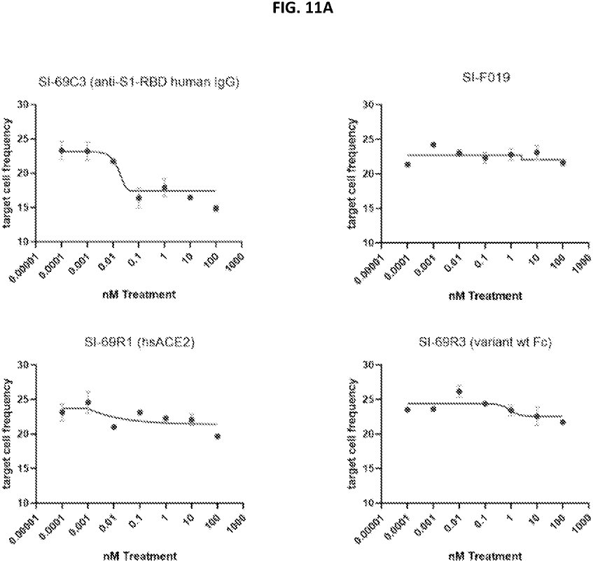

HEK293-T cells expressing either SARS-CoV-2 spike protein and the parental HEK293 cells were stained with the indicated materials for 30 minutes at 37° C. in the presence of internalization inhibitor sodium azide. After the removal of free SI-F019, SI-F109 was detected and quantified by using anti-human Fc AF647 fab (SKU109-607-008, Jackson ImmunoResearch) and flow cytometry analysis. Geometric mean signal intensity was used to quantify the binding of SI-F019 and target cells line as shown in FIG. 9. HEK293-T cells expressing either SARS-CoV-2 spike protein may serve as a model for COVID-19 infected cells.

### Example 11. The Effect of SI-F019 on Antibody-dependent Cellular Cytotoxicity (ADCC)

Antibody-dependent cellular cytotoxicity (ADCC) is one of important immune responses to viral infection, such as the infection of SARS-CoV-2 virus in the case of COVID-19. Following the initial viral infection, anti-virus antibodies directly bind to the viral particles for neutralization and agglutination. Binding of a virus-antibody complex to an Fc receptor on a phagocyte can trigger phagocytosis, resulting in destruction of the virus; binding to the Fc receptors on immune effector cells, such as monocytes, neutrophils, eosinophils and NK cells, can trigger the release of cytotoxic factors (e.g., antiviral interferons), creating a microenvironment that is hostile to virus replication.

To distinguish the effect of SI-F019 from anti-spike antibodies, HEK293-T cells expressing SARS-CoV-2 spike protein were loaded with Calcein-AM and co-cultured with purified human NK cells at a 5:1 effector to target ratio. Treatments tested included SI-F019 and S1-specific human IgG clone SI-69C3. SI-69C3 is the human antibody clone CC12.3, isolated from a hospitalized COVID-19 patient (10.1126/science.abc7520). After 12 hours in co-culture, cells were stained with propidium iodide and evaluated for viability. As shown in FIG. 10, reduction in the viable target cell frequency (Population 3) based on expression of Calcein-AM and Propidium Iodide staining was evaluated as a measure of cytolysis.

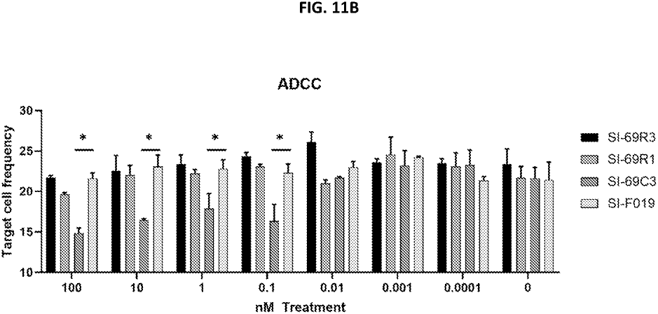

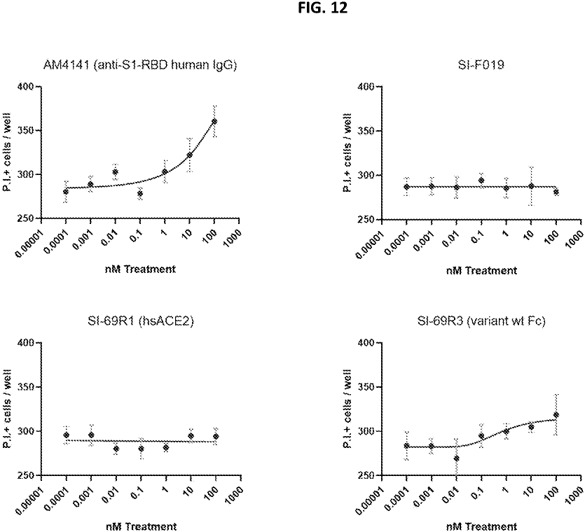

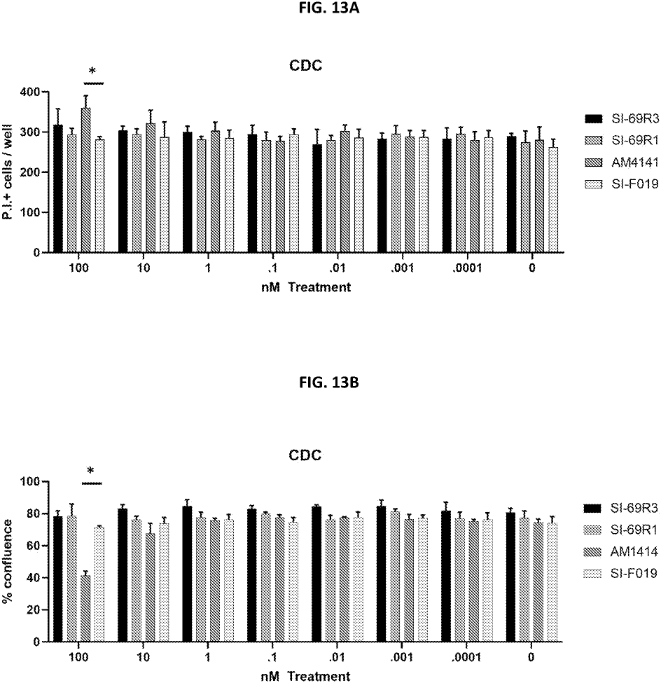

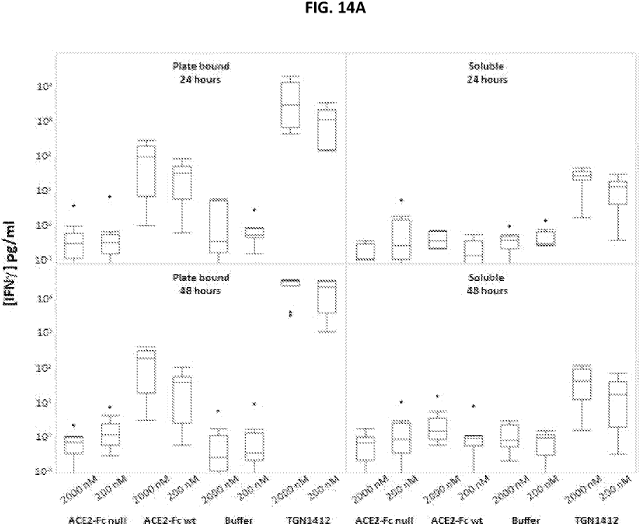

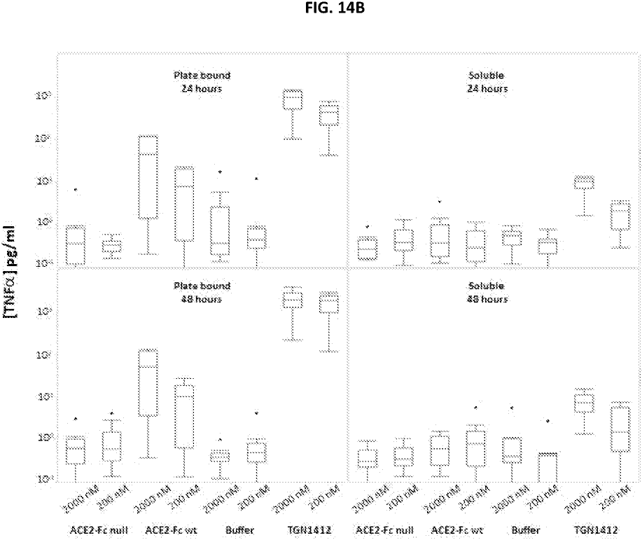

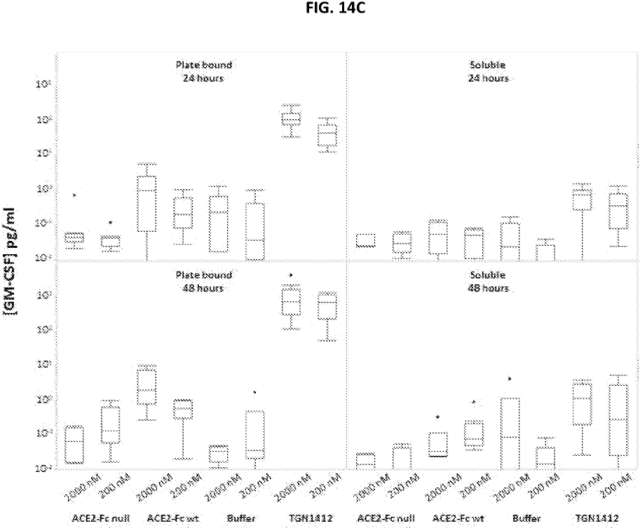

ADCC mediated by NK cells can be directed toward HEK293-T cells expressing SARS-CoV-2 protein when exposed to S1-specific human IgG clone SI-69C3 (Clone CC12.3). SI-F019 did not mediate ADCC compared to SI-69C3 within the treatment range of 100 nM to 100 fM. Under these assay conditions, the SI-F019 drug variant with wt Fc (SI-69R3) was able to mediate ADCC in a dose-dependent fashion, but the level of activity was lower compared to the S1-specific human IgG clone CC12.3 as shown in FIGS. 11 and FIG. 12. These data indicate that unlike SARS-CoV-2 S1 specific human IgG antibodies, SI-F019 does not mediate NK cell-mediated ADCC.

### Example 12. The Effect of SI-F019 on Complement-Dependent Cytotoxicity (CDC)

The role of the complement cascade in mediation of antibody-based cell and tissue injury in COVID-19 patients is evident in both the natural immune responses and neutralizing antibody-based therapy (Perico et al., 2021). Immune complexes formed of virus and specific IgG mediate complement-induced blood clotting, thromboembolism and systemic microangiopathy. These widespread complications in COVID-19 patients can be life-threatening and are dependent on the complement proteins binding to IgG. Virus immune complexes bridging red blood cells through C1q and platelets with FcγRIIA are mediators of the thromboembolism in COVID-19 patients (Nazy et al., 2020). The fixation of immune complexes to endothelial vessel walls and complement-mediated coagulation are a primary concern in patients with COVID-19 where the activation of endothelial cells is part of the thromboembolism cascade.

Unlike a natural IgG antibody, SI-F019 is unable to binding C1q as shown in Table 3. This feature eliminates the risk of the induction of cell death of infected epithelia and endothelium that may transiently express the SARS-CoV-2 spike protein on their surface. This protective effect of SI-F019 is demonstrated in comparison to anti-spike human IgG antibody.

To demonstrate the protective effect of SI-F019, HEK293-T cells expressing SARS-CoV-2 Spike protein were cultured in serum-free media (Optimem) with treatments for 30 minutes, followed by addition of human serum complement at 1:10 serum-to-media ratio. Treatments tested included SI-F019 and S1-specific human IgG clone AM001414 (BioLegend). Cell were cultured at 37° C. for 3 hours prior to addition of Propidium Iodide staining and positive staining cells counted in each well. Red cells counted by Incucyte Zoom Software at 3 hours are evaluated as a measure of CDC as shown in FIG. 12 and FIGS. 13. Total cellular confluence was evaluated after 96 hours as a measure of the impact of CDC as displayed in FIGS. 13.

The protection of tissue cells from complement damage is further confirmed by the ability of these cells to further proliferate after human serum complement challenge. CDC mediated by human serum complement at a 1:10 volume to volume ratio with serum free media is evaluable toward HEK293T cells expressing SARS-Cov-2 S protein when exposed to S1-specific human IgG clone (Clone AM4141). The result indicated that both human soluble monomeric ACE2 and SI-F019 did not mediate CDC, whereas SI-69R3 had limited, dose dependent increase CDC activity compared to human IgG antibody. CDC cytolysis was reflected in reduced cell growth, based on well confluence at 96 hours post treatment.

### Example 13 Cytokine Release Elicited by Soluble or Plate-Bound SI-F019 in PBMC Culture

SARS-CoV-2 has a tropism for ACE2-expressing epithelia of respiratory tract and small intestine. Clinical laboratory findings of elevated IL-2, IL-6, IL-7, granulocyte-macrophage colony-stimulating factor (GM-CSF), interferon-γ inducible protein 10 (IP-10), monocyte chemoattractant protein 1 (MCP-1), macrophage inflammatory protein 1-α (MIP-1α), and tumor necrosis factor-α (TNF-α) indicative of cytokine release syndrome (CRS) suggest an underlying immunopathology. CRS is a major adverse side effect that can limit the utility of treatment with biologics and is tested for using in vitro cytokine release assays.

SI-F019 is a fusion protein consisting of human ACE2 and a mutated form of human IgG1 Fc that is incapable of binding to Fcy receptors. As such, SI-F019 is not expected to bind any target cells in peripheral blood or to elicit cytokine release. White blood cells (WBC) including neutrophils, isolated from 5 healthy donors were put in culture wells containing either plate-bound or soluble SI-F019 at 2000 nM and 200 nM concentrations.

As a positive control, the TGN1412 antibody was used at the same concentrations and in the same formats due to its well-documented ability to induce cytokine release in the plate-bound format of this assay. The potential contribution of the IgG1 Fc null fragment to reduce cytokine release was evaluated by comparison with SI-69R3 having a wild type Fc fragment that is capable of binding Fcy receptors expressed by several cell types in peripheral blood. WBC cultures containing only the formulation buffer for SI-F019 at similar dilutions were used as a negative control. Culture supernatants were collected at 24 and 48-hour time points and the presence of 9 cytokines was detected using the Meso Scale Discovery (MSD) platform.

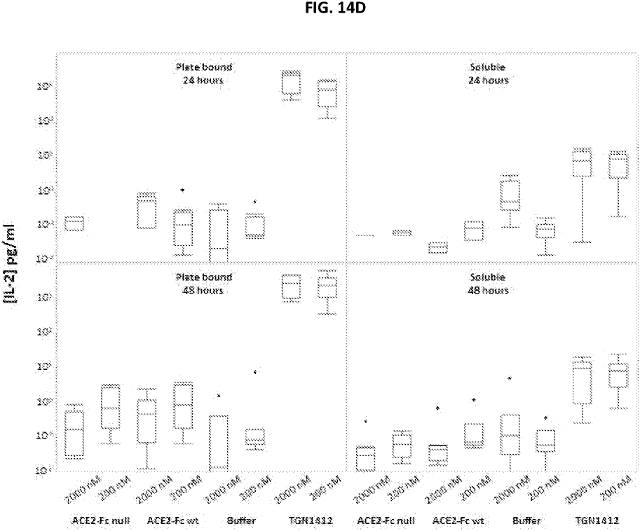

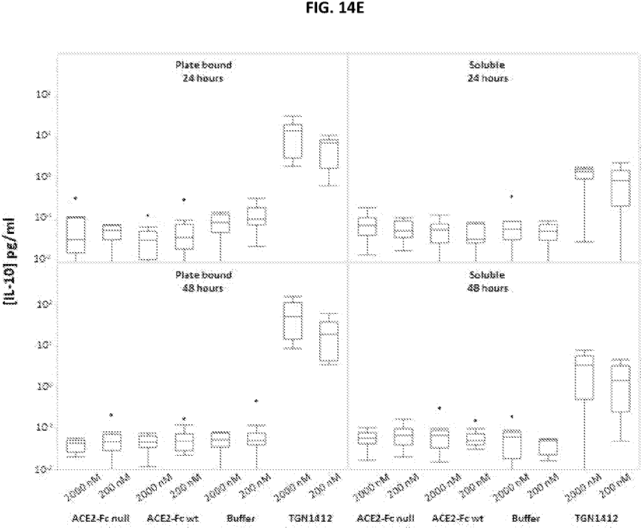

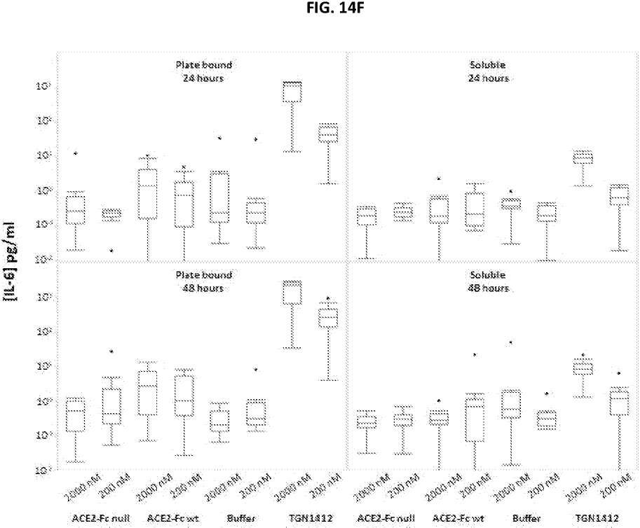

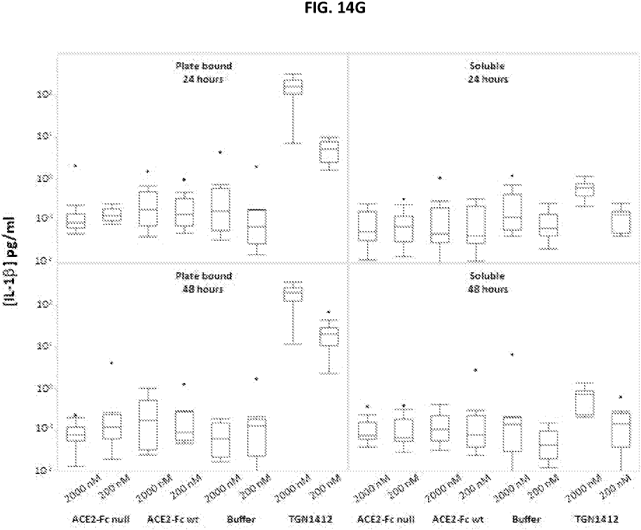

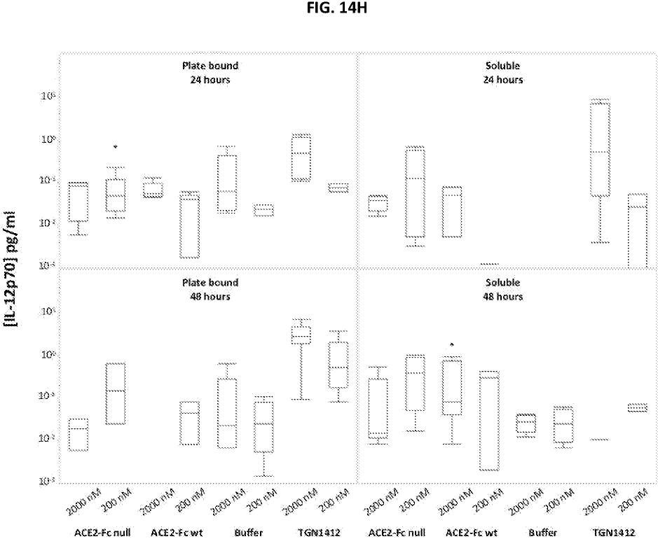

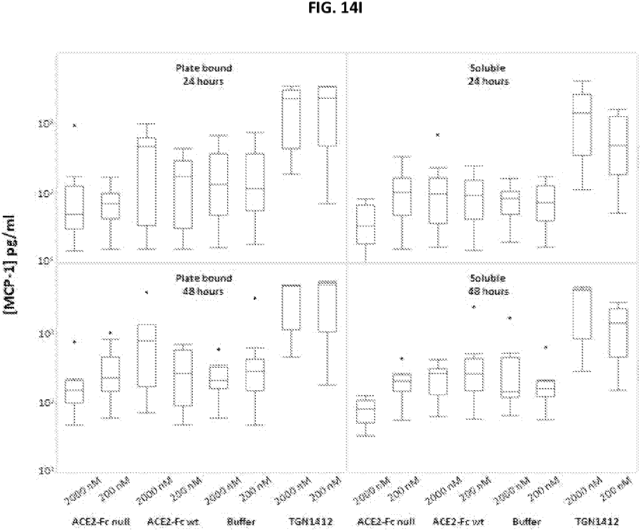

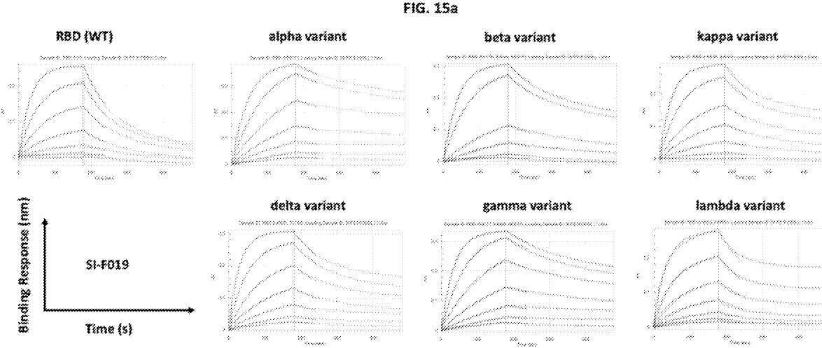

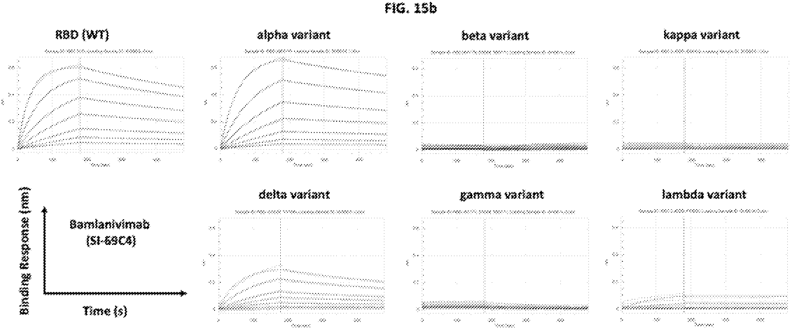

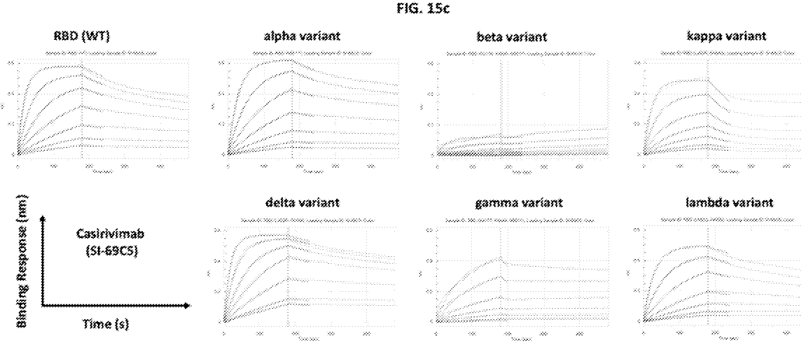

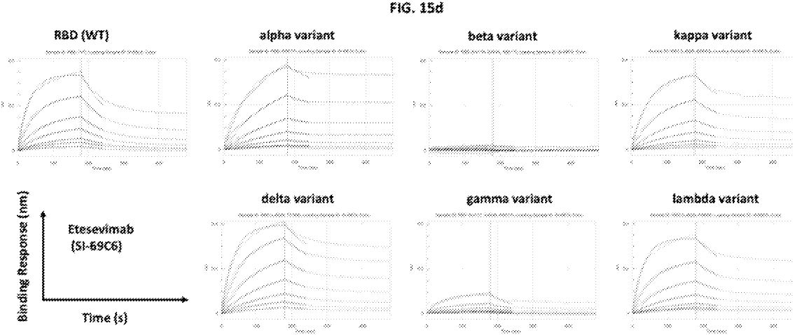

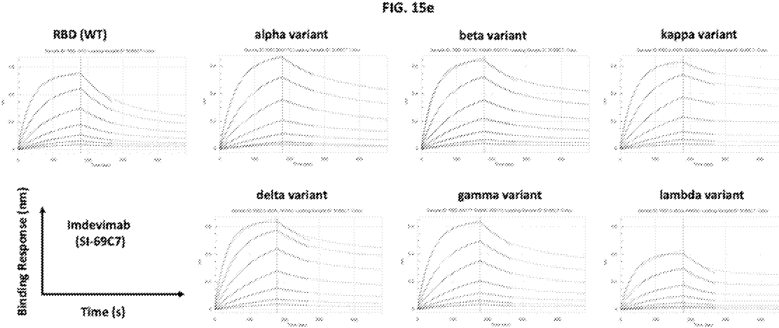

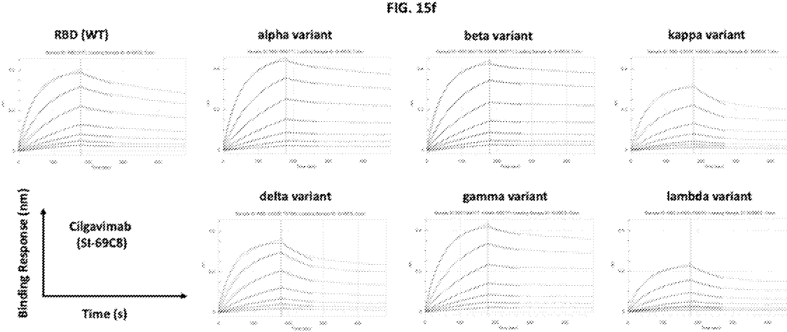

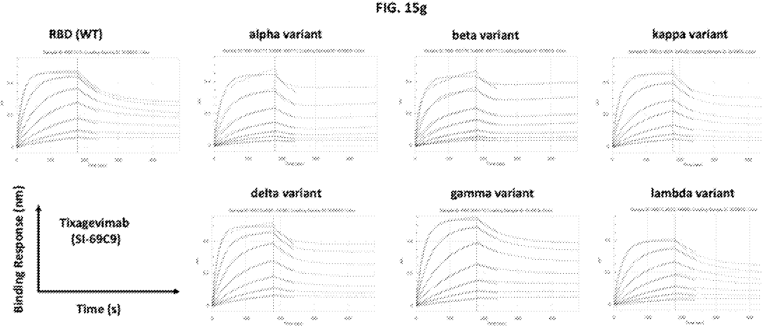

Included in the cytokine panel were the T cell-associated cytokines IFNγ, TNFα, GM-CSF, IL-2 and IL-10 as shown in FIGS. 14A-14E. Also tested were levels of the proinflammatory, non-T cell associated cytokines IL-1β, IL-12p70 and IL-6, as well as the monocyte chemoattractant protein, MCP-1 as shown in FIGS. 14F-14I. Results from duplicate wells for each blood donor were averaged and plotted using JMP14 software in box plots showing the 95% confidence intervals and outliers.

The results indicate that SI-F019 does not induce any of the tested cytokines from exposed to WBC in either plate-bound or soluble formats at 200 nM and 2000 nM concentrations. Cytokine levels in SI-F019 treated samples showed concentrations similar to buffer controls in all conditions. The positive control, TGN1412 strongly induced most of the cytokines in the plate-bound but not the soluble format, which is in an agreement with previously published results. Some intermediate production of IFNγ, GM-CSF, and TNFα were detectable when plate-bound ACE2-Fc wild type was used to stimulate the WBC indicating the increased safety of the Fc null fragment of SI-F019.

The pathogenic role of the humoral response against SARS-CoV-2 virus has recently been suggested in patients receiving interventional IgG therapy (Weinreich et al., 2021; Chen et al., 2021). The small vessel hyperinflammatory response underlies adverse events, including thrombocytosis, pruitus, pyrexia, and hypertension. The present application demonstrates that SI-F019 could provide the benefit of virus neutralization comparable to that of IgG therapy while protecting tissues and organs from multiple pathways of dysfunctions. Therefore, SI-F019 may be used for treating, preventing, or moderating a viral infection, specifically for preventing and managing the progression of COVID-19 with reduced clinical complications, and additionally for acute respiratory distress syndrome, pulmonary arterial hypertension, or acute lung injury.

### Example 14. Binding Kinetics (Affinity) of SI-F019 to SARS-CoV-2 RBD Variants

As the pandemic continues, mutation and selection drive the evolution of SARS-CoV-2 viruses to gain higher binding affinity to ACE2 for a higher rate of viral transmission, which result in mutant strains including the newly emerged and highly contagious Delta variant. Indeed, both Alpha variant and the Delta variant are more transmissible than the original SARS-CoV-2 virus. The prevalence of SARS-CoV-2 variants is the unmet challenge for developing treatment and prophylaxis. While SI-F019 is a candidate neutralizing agent, FDA has approved several neutralizing antibodies for treating patients, including b) Bamlanivimab (Eli Lilly’s LY-CoV555; SI-69C4, SEQ ID No. 29 and 30); c) Casirivimab (Regeneron’s REGN10933; SI-69C5, SEQ ID NO. 31 and 32); d) Etesevimab (Eli Lilly’s CB-6; SI-69C6, SEQ ID NO. 33 and 34); e) Imdevimab (Regeneron’s REGN10987; SI-69C7, SEQ ID NO. 35 and 36); f) Cilgavimab (AstraZeneca’s AZD1061; SI-69C8, SEQ ID NO. 37and 38); and g) Tixagevimab (AstraZeneca’s AZD8895; SI-69C9, SEQ ID NO. 39 and 40).

To determine the comparative advantage of SI-F019, a recombinant ACE2-Fc fusion protein, with those neutralizing antibodies, Bio-Layer interferometry was used to quantify the strength of their binding interactions to SARS-CoV-2 RBD variants mimicking COVID-19 variants by using an Octet Red 384. These variant proteins were purchased from Sino Biological. First, 10 µg/ml of SI-F019 protein in assay buffer (PBS containing 1% BSA and 0.05% Tween 20) was loaded onto AHC sensors for 180 seconds. After a 180-second baseline step, loaded protein was allowed to associate with 1:2 serial dilutions (top concentration from 50 nM) of RBD variant protein in assay buffer for 180 seconds, followed by a 300-second dissociation step in assay buffer. Regeneration was performed using 10 mM glycine pH 1.5. Data were globally fit to a 1:1 binding model using the full association phase and the first 60 seconds of the dissociation phase, in order to extract kinetic parameters K_{D}, kₒₙ, and k_{dis}. The binding affinity of the same variants and wild type RBD proteins as shown in Table 2 and Table 4 are comparable, as the different readouts may result from different vendors (ACROBiosystem and Sino Biological).

FIGS. 15(a-g) shows sensorgrams of SI-F019 and neutralizing antibodies while binding to RBD variants, and Table 4 (a-g) tabulates the extracted binding kinetics (affinity) parameters. The results show that as compared to SI-F019 (15a), Bamlanivimab (SI-69C4) was unable to bind to the Kappa, Gamma, Beta and Lambda variants (15b); Casirivimab (SI-69C5) displayed a significant decrease in its binding affinity towards Gamma and Beta variants (15c); and Etesevimab (SI-69C6) also displayed a significant decrease in its binding affinity towards Gamma and Beta variants (15d). While other monoclonal antibodies, such as Imdevimab (SI-69C7)(4e), Cilgavimab (SI-69C8)(4f), and Tixagevimab (SI-69C9)(4 g), may not show any significant changes in their binding affinity, the sensorgrams unveiled decreased binding response in some of the variants (FIGS. 15e, 15f, and 15g). In contrast to neutralizing antibodies, SI-F019 exhibited improved binding affinity to emerging variants, including the highly contagious Delta variant, as to the ancestral wild-type RBD (FIG. 15a, Table 4a). The observation supports the notion that the ACE2 portion of SI-F019 retains the conformation of wild type full length ACE2 for viral entry into human cells, which is the target for the selection of high affinity variants. Therefore, SI-F019 has the comparative advantage with those FDA-approved neutralizing antibodies, thereby can be used as a binding and blocking agent.

### Example 15. Binding Kinetics (Avidity) of SI-F019 and Neutralizing Antibodies to Variants

While the binding affinity assay above measures the binding of SI-F019 immobilized on the anti-human IgG Fc Capture Biosensors tip (AHC) surface to SARS-CoV-2 RBD protein variants in solution, the avidity assay measures the binding of a biotinylated SARS-CoV-2 RBD protein variants immobilized on the Streptavidin Biosensors tip surface to SI-F019 in solution.

Bio-Layer interferometry was used to quantify the strength of binding interactions between SI-F019 and SARS-CoV-2 S protein variant RBD domains using an Octet Red 384. The reagents were purchased from Sino Biological and chemically biotinylated by NHS-ester activated reaction, with the stoichiometric ratio of biotin/protein is 2:1. First, 2 µg/ml of biotinylated RBD or its variant protein in assay buffer (PBS containing 1% BSA and 0.05% Tween 20) was loaded onto SA sensors for 180 seconds. After a 180-second baseline step, loaded protein was allowed to associate with 1:2 serial dilutions (top concentration from 50 nM) of SI-F019 protein (GMP lot) in assay buffer for 300 seconds, followed by a 600-second dissociation step in assay buffer. Regeneration was performed using 10 mM glycine pH 1.5. Data were globally fit to a 1:1 binding model using the full association phase and the full dissociation phase, in order to calculate kinetic parameters K_{D}, kₒₙ, and k_{dis}. The binding affinity of the same variants and wild type RBD proteins as shown in Table 2 and Table 5 are comparable, as the different readouts may result from different vendors (ACROBiosystem and Sino Biological).

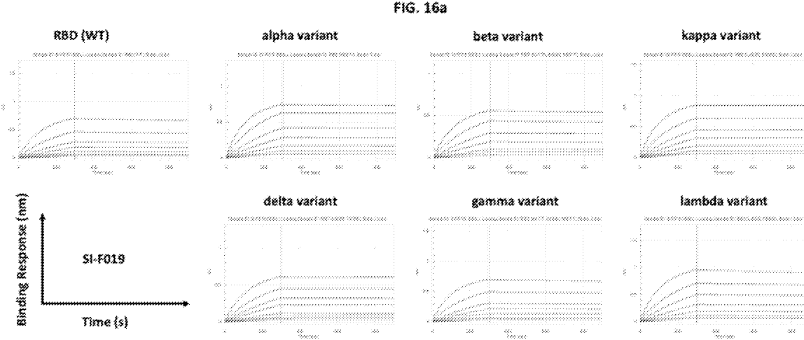

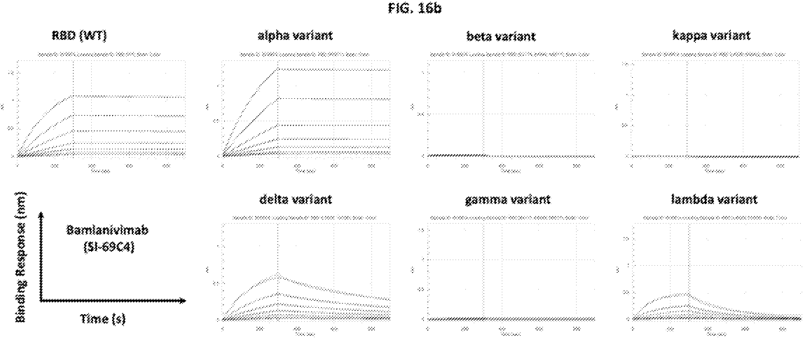

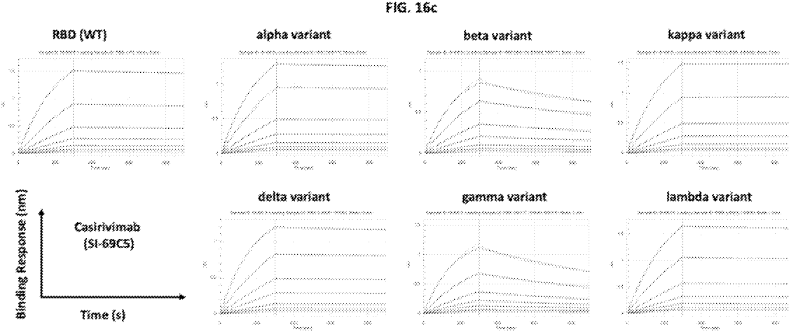

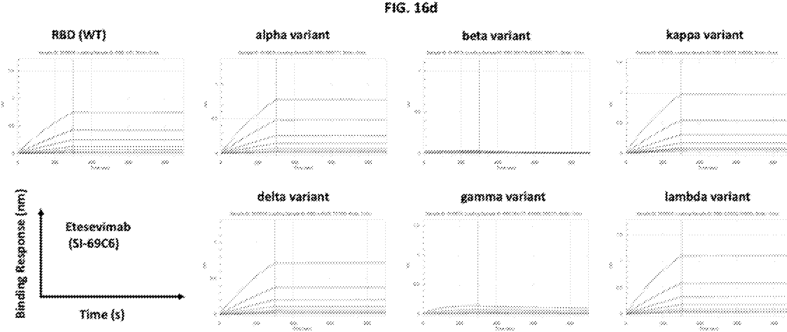

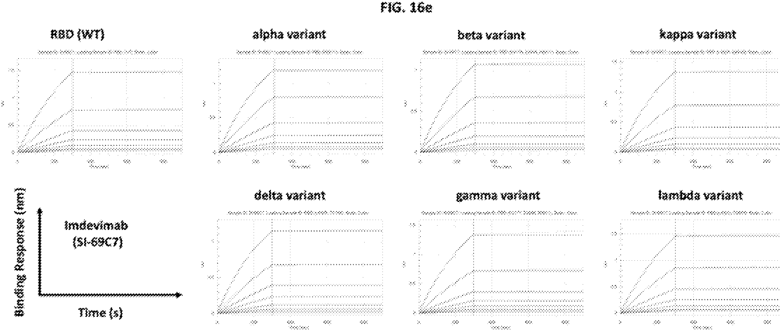

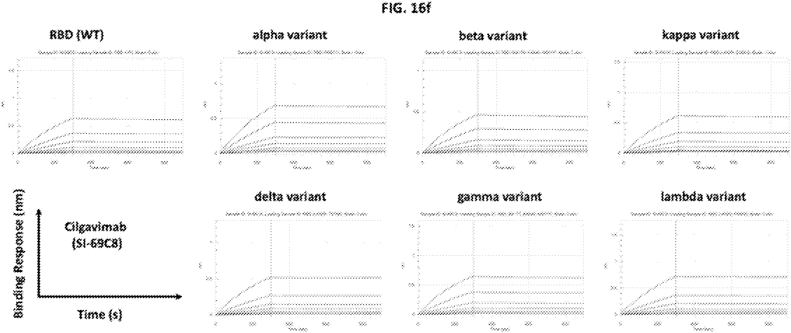

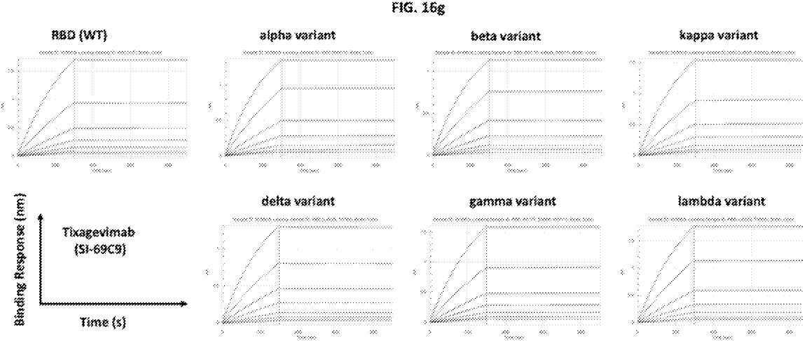

FIGS. 16(a-g) shows sensorgrams of SARS-CoV-2 RBD protein variants binding to SI-F019 and Table 5 (a-g) tabulates the extracted binding kinetics (avidity) parameters. Notably, SI-F019 binds with higher avidity to variant forms of RBD relative to the wild-type RBD, with increased avidity driven largely by slower dissociation rate. The results show increasing binding affinity and decreasing dissociation rate of SI-F019 to RBD variants relative to wild-type RBD; and reduced or diminished effectiveness in some neutralizing antibodies.

### Example 16. Comparative Advantage of SI-F019 With Neutralizing Antibodies

To demonstrate the comparative advantage of SI-F019 with neutralizing antibodies, the values of binding response or Response are used. Response is measured as a nm shift in the interference pattern as shown in FIGS. 15, 16, and is proportional to the number of molecules bound to the surface of the biosensor. Response is the maximum binding signal achieved when associating with the highest concentration of analyte, and is a complex function that depends on kinetic binding parameters, protein sizes, and assay conditions, and should be comparable in a given assay as long as the interacting proteins are of similar size and format. Differences in response can indicate differences in the effective strength of a protein-protein interaction, where high response suggests a strong interaction and low response suggests a weaker interaction.

Table 6 tabulated the extracted values of Response (highest concentration of analyte) from the binding affinity and avidity of SI-F019 and neutralizing antibodies in FIGS. 15, and 16. To evaluate the change of binding kinetics for each neutralizing agent and variant, these definitions applied: 1) when Response <10% of WT, no binding; 2) when Response <30% of WT, minimal binding; 3) when Response <75% of WT, low binding; and 4) for complex kinetics, including upward dissociation, non-specific binding. When these criteria were applied to each Response, each neutralizing antibody shows low to no binding to at least one variant. In particular for the avidity assay, Bamlanivimab (SI-69C4) had reduced binding response to Delta, Kappa, Gamma, Beta, and Lambda; Etesevimab (SI-69C6) had reduced binding response to Gamma and Beta; and Casirivimab (SI-69C5), Imdevimab (SI-69C7), Cilgavimab (SI-69C8), and Tixagevimab (SI-69C9) all had reduced binding response to Beta. In contrast, SI-F019 retains its binding affinity and avidity to all variants.

### Example 17. SI-F019 as a Blocking Agent Capable of Inhibiting Viral Infectivity of COVID-19 RBD Variants

To test the ability of SI-F019 to prevent viral infection, viral infectivity was characterized using a luciferase reporter assay. SARS-CoV-2 S protein packaged pseudovirus (wild-type or variant strains, Sino Biological) containing a luciferase reporter gene (NICPBP) was co-incubated with 293T cells overexpressing ACE2 (clone 3D4) and 1:3 serial dilutions (from 30 µg/ml) of SI-F019. Expression of ACE2 on the transfected cells was confirmed by enzymatic and FACS assays. The pseudovirus may enter the ACE2-positive cells via S protein binding to ACE2, which leads to expression of luciferase. Thus, luminescence is used as a readout of viral infectivity.

In particular, 10x stock solution of S protein pseudovirus was prepared in culture medium to a final virus load of 227-394 TCID50/well. SI-F019 in culture medium was serially diluted 3-fold with maximum concentration 150 µg/ml (final 30 µg/ml). 3D4 cells were harvested using dissociation buffer lacking trypsin. Pseudovirus (20 µl) and SI-F019 (30 µl) were combined in wells of a 96-well plate, mixed, and incubated for 1 hour at room temperature. Then, 100 µl of harvested 3D4 cells were added to each well (20,000/well) and incubated for 18 hours at 37° C., 5% CO₂. After incubation, supernatant was removed and 50 µl of luciferase substrate solution was added, mixed, and incubated for 1 minute at room temperature. Luminescence was read using I3X plate reader, where the luminescence signal in RLU (relative luminescence units) is representative of S protein pseudovirus infectivity.

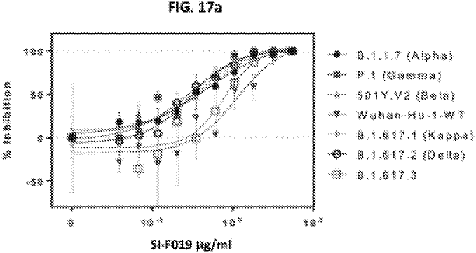

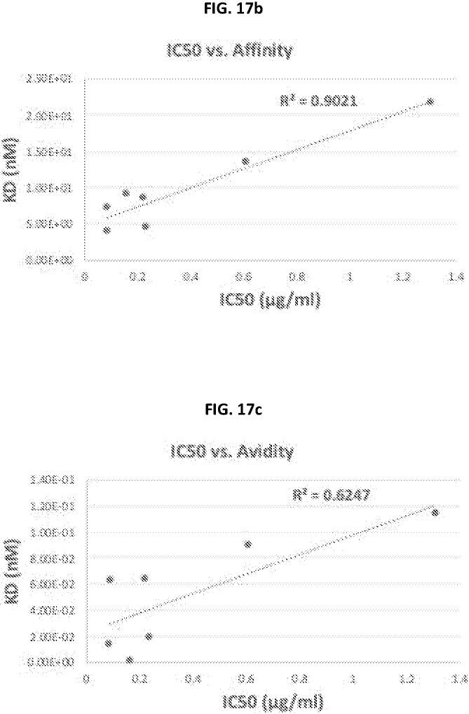

Decrease in luminescence compared to the condition without SI-F019 can be calculated to determine percent inhibition of infectivity. This data was then fit to a sigmoidal function in GraphPad Prism 6.0 to extract IC50 values for SI-F019 inhibiting pseudovirus infectivity where the pseudovirus contained different variants of S protein. Viral inhibition data are plotted in FIG. 17a and IC50 values are tabulated in Table 7. The results show that SI-F019 is capable of inhibiting the infectivity of all variants with an increased potency from 2 to 15-fold when compared to its potency to wild-type strain and that the linear association between IC50 and KD values of either affinity or avidity (FIGS. 17b, 17c) is indicative of a competitive inhibition by SI-F019. The in vitro experimental data demonstrates that ACE2 protein, such as SI-F019 fusion protein as to the membrane-bound ACE2 protein, is capable of not only tightly binding to but also inhibiting variants of SARS-CoV-2 virus with an increased affinity of the RBD-ACE2 interaction that evolves with increased infectivity and transmissibility.

### Example 18. SI-F019 in a Phase I Clinical Trial in Healthy Participants in China (NCT04851444)

Three anti-SARS-CoV-2 monoclonal antibody products currently have Emergency Use Authorizations (EUAs) from the Food and Drug Administration (FDA) for the treatment of mild to moderate COVID-19 in non-hospitalized patients with laboratory-confirmed SARS-CoV-2 infection who are at high risk for progressing to severe disease and/or hospitalization. First, Bamlanivimab plus Etesevimab: these are neutralizing monoclonal antibodies that bind to different but overlapping epitopes in the spike protein RBD of SARS-CoV-2; second, Casirivimab plus Imdevimab: these are recombinant human monoclonal antibodies that bind to nonoverlapping epitopes of the spike protein RBD of SARS-CoV-2; and third, Sotrovimab: this monoclonal antibody was originally identified in 2003 from a SARS-CoV survivor. It targets an epitope in the RBD of the spike protein that is conserved between SARS-CoV and SARS-CoV-2.

Unlike those monoclonal antibodies that bind to a single epitope, SI-F019 has the technical advantage of competing with the ACE2 protein on human cells for binding to all docking sites of the spike protein RBD of SARS-CoV-2, some of which may overlap with those epitopes. Reducing the burden and technical difficulties of combining two or more monoclonal antibodies as a therapeutic regimen, SI-F019, which is currently in clinical trials, has the advantage of acting as a single effective therapeutic agent for treating mild to moderate COVID-19 and for post-exposure prophylaxis (PEP) of SARS-CoV-2 infection in individuals who are at high risk for progression to severe COVID-19. SI-F019 is capable of competing with other coronaviruses that also target the membrane-bound ACE2 protein on human cells, such as SARS-CoV-1 (FIG. 4). In addition to COVID-19, SI-F019 may be used to treat severe acute respiratory syndrome (SARS) caused by SARS-CoV-1 virus, Middle East respiratory syndrome (MERS) caused by MERS-CoV virus, as well as acute respiratory distress syndrome (ARDS) and other injuries to lung. In those cases, SI-F019 may also be used in combination with other therapeutic agents as needed.

The purpose of the Phase-I trial is to test the safety, tolerability, and pharmacokinetic properties of a single intravenous administration of SI-F019. The trial is designed in a double blind, placebo controlled and randomized manner with dose escalation from 3 mg/kg to 70 mg/kg SI-F019 (Table 8a, 8b). The fusion protein is administered as a liquid suspension in histidine/histidine hydrochloride, sodium chloride, sucrose and polysorbate 80. 36 participants in total were given a single dose at day 1 and followed up to day 29. Treatment emergent adverse event (TEAE), treatment related adverse event (TRAE), severity and laboratory abnormality were captured and graded by NCI-CTCAE v5.0. As of September 16^{th}, 2021, the electronic data capture (EDC) database has not been locked yet. Based on the blind data review, 21 out of 36 participants experienced 44 adverse events (AEs) among whom 31 TRAEs occurred in 16 participants. All AEs were grade 1 and no significant association with SI-F019 dose was found (Table 9a, 9b). The favorable overall tolerability and safety of SI-F019 support its further exploration as a prophylactic and therapeutic agent against COVID-19 and other related diseases.

### TABLES

TABLE 1

The cloning, expression, and purification of recombinant ACE2-Fc fusion proteins

| | | | |
|---|---|---|---|
| Recombinant fusion protein | Sample ID | | Purified fusion protein |
| huACE2 (1-615) - 6His-tagged | SI-69R1 | | huACE2 (18-615) - 6His-tagged |
| huACE2 (1-615) - IgG1 Fc (w2) | SI-69R3 | | huACE2 (18-615) - IgG1 Fc (w2) |
| huACE2 (1-740) - IgG1 Fc (w2) | SI-69R4 | | huACE2 (18-740) - IgG1 Fc (w2) |
| huACE2 (1-615) - IgG1 Fc null | SI-69R2 | SI-F019 | huACE2 (18-615) - IgG1 Fc null |
| huACE2 (1-615) - IgG4 Fc | SI-69R2-G4 | | huACE2 (18-615) - IgG4 Fc |
| huACE2 (1-615) - IgA1 Fc | SI-69R2-A1 | | huACE2 (18-615) - IgA1 Fc |
| huACE2 (1-615) - IgA2 Fc | SI-69R2-A2 | | huACE2 (18-615) - IgA2 Fc |

TABLE 2

The affinity and avidity of SI-F019 binding to viral proteins

| | Affinity KD (nM) | Kon (1/ms) | Kdis (1/s) | Avidity KD (nM) | Kon (1/ms) | Kdis (1/s) |
|---|---|---|---|---|---|---|
| CoV2 S1 | 14.7 | 3.37E+05 | 4.93E-03 | 0.29 | 1.49E+05 | 4.72E-05 |
| CoV2 RBD | 13.8 | 3.45E+05 | 4.79E-03 | 0.89 | 3.09E+05 | 2.70E-04 |
| CoV RBD | 14.0 | 4.26E+05 | 5.97E-03 | 0.33 | 5.18E+05 | 1.71E-04 |
| CoV2 SPIKE Trimer | NA | NA | NA | 0.18 | 4.20E+04 | 1.12E-05 |

TABLE 3

The effect of Fc null mutations on its binding to Fc receptors

| Fc Receptor | KD (nM) | Kon (1/Ms) | Kdis (1/s) |
|---|---|---|---|
| FcγRI/CD64 | Not detectable | | |
| FcγRIIa/CD32a | Not detectable | | |
| FcγRIIb/CD32b | Not detectable | | |
| FcγRIIIa/CD16a | Not detectable | | |
| C1q | Not detectable | | |
| FcRn | 37.6 | 4.51E+05 | 3.52E-0.2 |

Binding kinetics (affinity) of SI-F019 (4a) and neutralizing antibodies to different variants of S protein RBD, including Bamlanivimab (SI-69C4)(4b); Casirivimab (SI-69C5)(4c); Etesevimab (SI-69C6)(4d); Imdevimab (SI-69C7)(4e); Cilgavimab (SI-69C8)(4f); and Tixagevimab (SI-69C9)(4 g), indicating that SI-F019 binds with increased affinity to variant forms of RBD relative to the wild-type RBD, driven largely by slower dissociation rate, while at least three neutralizing antibodies lost their binding to at least one variant (N.D.).

TABLE 4

| WHO Designation | RBD Mutation | 4a. SI-F019 | | |
|---|---|---|---|---|
| | | KD (M) | Kon (1/Ms) | Kdis (1/s) |
| Original | WT | 2.18E-08 | 4.26E+05 | 9.26E-03 |
| Alpha | N501Y | 4.54E-09 | 3.96E+05 | 1.80E-03 |
| Delta | L452R, T478K | 7.26E-09 | 6.11E+05 | 4.44E-03 |
| Kappa | L452R, E484Q | 9.07E-09 | 5.12E+05 | 4.65E-03 |
| Gamma | K417T, E484K, N501Y | 4.03E-09 | 5.30E+05 | 2.13E-03 |
| Beta | K417N, E484K, N501Y | 8.49E-09 | 4.24E+05 | 3.61E-03 |
| Lambda | L452Q, F490S | 1.35E-08 | 4.59E+05 | 6.21E-03 |

Continued-1

| WHO Designation | 4b. Bamlanivimab (SI-69C4) | | | 4c. Casirivimab (SI-69C5) | | |
|---|---|---|---|---|---|---|
| | KD (M) | Kon (1/Ms) | Kdis (1/s) | KD (M) | Kon (1/Ms) | Kdis (1/s) |
| Original | 1.64E-09 | 5.86E+05 | 9.60E-04 | 2.02E-09 | 9.75E+05 | 1.96E-03 |
| Alpha | 1.67E-09 | 4.03E+05 | 6.74E-04 | 2.65E-09 | 6.59E+05 | 1.75E-03 |
| Delta | 3.61E-09 | 3.52E+05 | 1.27E-03 | 1.02E-09 | 1.14E+06 | 1.16E-03 |
| Kappa | N.D. | N.D. | N.D. | 7.20E-09 | 7.90E+05 | 5.69E-03 |
| Gamma | N.D. | N.D. | N.D. | 2.93E-08 | 1.92E+05 | 5.64E-03 |
| Beta | N.D. | N.D. | N.D. | N.D. | N.D. | N.D. |
| Lambda | N.D. | N.D. | N.D. | 2.52E-09 | 6.50E+05 | 1.64E-03 |

Continued-2

| WHO Designation | 4d. Etesevimab (SI-69C6) | | | 4e. Imdevimab (SI-69C7) | | |
|---|---|---|---|---|---|---|
| | KD (M) | Kon (1/Ms) | Kdis (1/s) | KD (M) | Kon (1/Ms) | Kdis (1/s) |
| Original | 1.67E-08 | 4.89E+05 | 8.16E-03 | 1.10E-08 | 4.51E+05 | 4.98E-03 |
| Alpha | 9.59E-09 | 2.41E+05 | 2.31E-03 | 6.53E-09 | 3.81E+05 | 2.48E-03 |
| Delta | 8.09E-09 | 4.93E+05 | 3.99E-03 | 4.77E-09 | 5.61E+05 | 2.68E-03 |
| Kappa | 1.48E-08 | 3.18E+05 | 4.72E-03 | 3.34E-09 | 4.83E+05 | 1.61E-03 |
| Gamma | 4.68E-08 | 2.44E+05 | 1.14E-02 | 7.72E-09 | 4.40E+05 | 3.40E-03 |
| Beta | N.D. | N.D. | N.D. | 6.01E-09 | 4.90E+05 | 2.94E-03 |
| Lambda | 8.24E-09 | 5.61E+05 | 4.62E-03 | 1.10E-08 | 4.48E+05 | 4.91E-03 |

Continued-3

| WHO Designation | 4f. Cilgavimab (SI-69C8) | | | 4g. Tixagevimab (SI-69C9) | | |
|---|---|---|---|---|---|---|
| | KD (M) | Kon (1/Ms) | Kdis (1/s) | KD (M) | Kon (1/Ms) | Kdis (1/s) |
| Original | 3.98E-09 | 4.34E+05 | 1.72E-03 | 3.63E-09 | 1.12E+06 | 4.06E-03 |
| Alpha | 2.44E-09 | 4.01E+05 | 9.81E-04 | 3.17E-09 | 8.21E+05 | 2.60E-03 |
| Delta | 7.34E-09 | 4.38E+05 | 3.21E-03 | 3.83E-09 | 1.12E+06 | 4.30E-03 |
| Kappa | 9.89E-09 | 3.68E+05 | 3.64E-03 | 5.21E-09 | 9.00E+05 | 4.69E-03 |
| Gamma | 2.82E-09 | 3.92E+05 | 1.10E-03 | 5.83E-09 | 8.30E+05 | 4.84E-03 |
| Beta | 1.43E-09 | 4.41E+05 | 6.31E-04 | 3.24E-09 | 9.81E+05 | 3.18E-03 |
| Lambda | 9.81E-09 | 3.54E+05 | 3.48E-03 | 4.31E-09 | 7.82E+05 | 3.37E-03 |

Biolayer interferometry was used to quantify binding kinetics (avidity) of SI-F019 (5a) and neutralizing antibodies to different variants of S protein RBD, including Bamlanivimab (SI-69C4)(5b); Casirivimab (SI-69C5)(5c); Etesevimab (SI-69C6)(5d); Imdevimab (SI-69C7)(5e); Cilgavimab (SI-69C8)(5f); and Tixagevimab (SI-69C9)(5 g).

TABLE 5

| WHO Designation | RBD Mutation | 5a. SI-F019 | | |
|---|---|---|---|---|
| | | KD (M) | Kon (1/Ms) | Kdis (1/s) |
| Original | WT | 1.14E-10 | 8.63E+05 | 9.82E-05 |
| Alpha | N501Y | 1.90E-11 | 1.02E+06 | 1.94E-05 |
| Delta | L452R, T478K | 1.41E-11 | 8.58E+05 | 1.21E-05 |
| Kappa | L452R, E484Q | <1.0E-12 | 1.01E+06 | <1.0E-07 |
| Gamma | K417T, E484K, N501Y | 6.23E-11 | 1.00E+06 | 6.26E-05 |
| Beta | K417N, E484K, N501Y | 6.35E-11 | 8.93E+05 | 5.66E-05 |
| Lambda | L452Q, F490S | 8.92E-11 | 9.13E+05 | 8.15E-05 |

Continued-1

| WHO Designation | 5b. Bamlanivimab (SI-69C4) | | | 5c. Casirivimab (SI-69C5) | | |
|---|---|---|---|---|---|---|
| | KD (M) | Kon (1/Ms) | Kdis (1/s) | KD (M) | Kon (1/Ms) | Kdis (1/s) |
| Original | 7.03E-11 | 5.60E+05 | 3.94E-05 | 1.37E-10 | 4.64E+05 | 6.34E-05 |
| Alpha | 5.55E-11 | 4.10E+05 | 2.27E-05 | 7.83E-11 | 5.17E+05 | 4.05E-05 |
| Delta | 2.37E-09 | 5.38E+05 | 1.27E-03 | 8.04E-11 | 5.54E+05 | 4.46E-05 |
| Kappa | N.D. | N.D. | N.D. | <1.0E-12 | 4.57E+05 | <1.0E-07 |
| Gamma | N.D. | N.D. | N.D. | 1.33E-09 | 5.64E+05 | 7.52E-04 |
| Beta | N.D. | N.D. | N.D. | 9.20E-10 | 5.60E+05 | 5.16E-04 |
| Lambda | 4.83E-09 | 8.46E+05 | 4.09E-03 | 9.58E-11 | 4.24E+05 | 4.07E-05 |

Continued-2

| WHO Designation | 5d. Etesevimab (SI-69C6) | | | 5e. Imdevimab (SI-69C7) | | |
|---|---|---|---|---|---|---|
| | KD (M) | Kon (1/Ms) | Kdis (1/s) | KD (M) | Kon (1/Ms) | Kdis (1/s) |
| Original | 4.92E-11 | 2.56E+05 | 1.26E-05 | <1.0E-12 | 3.06E+05 | <1.0E-07 |
| Alpha | 2.43E-11 | 2.26E+05 | 5.50E-06 | <1.0E-12 | 3.85E+05 | <1.0E-07 |
| Delta | <1.0E-12 | 2.56E+05 | <1.0E-07 | <1.0E-12 | 3.57E+05 | <1.0E-07 |
| Kappa | 2.96E-11 | 2.70E+05 | 7.99E-06 | <1.0E-12 | 3.44E+05 | <1.0E-07 |
| Gamma | 4.15E-10 | 1.39E+06 | 5.78E-04 | <1.0E-12 | 3.24E+05 | <1.0E-07 |
| Beta | N.D. | N.D. | N.D. | <1.0E-12 | 3.40E+05 | <1.0E-07 |
| Lambda | <1.0E-12 | 2.70E+05 | <1.0E-07 | <1.0E-12 | 2.73E+05 | <1.0E-07 |

TABLE 5

Continued-3

| WHO Designation | 5f. Cilgavimab (SI-69C8) | | | 5g. Tixagevimab (SI-69C9) | | |
|---|---|---|---|---|---|---|
| | KD (M) | Kon (1/Ms) | Kdis (1/s) | KD (M) | Kon (1/Ms) | Kdis (1/s) |
| Original | 2.10E-10 | 3.12E+05 | 6.53E-05 | 2.21E-11 | 3.35E+05 | 7.40E-06 |
| Alpha | 1.24E-10 | 3.44E+05 | 4.26E-05 | <1.0E-12 | 4.47E+05 | <1.0E-07 |
| Delta | 4.81E-11 | 3.35E+05 | 1.61E-05 | 3.03E-11 | 4.22E+05 | 1.28E-05 |
| Kappa | 2.04E-10 | 2.98E+05 | 6.08E-05 | <1.0E-12 | 4.16E+05 | <1.0E-07 |
| Gamma | 1.93E-10 | 3.85E+05 | 7.42E-05 | <1.0E-12 | 3.76E+05 | <1.0E-07 |
| Beta | 2.73E-10 | 3.55E+05 | 9.67E-05 | <1.0E-12 | 4.40E+05 | <1.0E-07 |
| Lambda | 8.15E-11 | 2.91E+05 | 2.37E-05 | <1.0E-12 | 3.62E+05 | <1.0E-07 |

Comparative analysis of the maximum binding response (affinity) by SI-F019 and neutralizing antibodies to SARS-CoV-2 viral RBD, indicating a range of low to no response by four of these antibodies to at least one variant, while SI-F019 shows increased response to all variants.

TABLE 6a

| | | | | | | | |
|---|---|---|---|---|---|---|---|
| WHO Designation | SI-F019 | SI-69C4 | SI-68C5 | SI-69C6 | SI-69C7 | SI-69C8 | SI-69C9 |
| Original | 0.255 | 0.6224 | 0.5842 | 0.3512 | 0.561 | 0.3973 | 0.4767 |
| Alpha | 0.3801 | 0.6714 | 0.6233 | 0.3763 | 0.6723 | 0.4521 | 0.5523 |
| Delta | 0.3115 | 0.326 | 0.5787 | 0.4078 | 0.6585 | 0.3566 | 0.5133 |
| | | Low | | | | | |
| Kappa | 0.2905 | 0.0516 | 0.509 | 0.3368 | 0.6459 | 0.3223 | 0.4717 |
| | | No | | | | | |
| Gamma | 0.3336 | 0.0624 | 0.4199 | 0.0923 | 0.6419 | 0.4283 | 0.4742 |
| | | Minimal | Low | Minimal | | | |
| Beta | 0.3046 | 0.031 | 0.139 | 0.0246 | 0.658 | 0.4384 | 0.4602 |
| | | No | Nonspecific | No | | | |
| Lambda | 0.278 | 0.1124 | 0.4976 | 0.3516 | 0.429 | 0.236 | 0.4113 |
| | | Minimal | | | | Low | |

Comparative analysis of the binding response (avidity) to SARS-CoV-2 viral RBD by SI-F019 or neutralizing antibodies, indicating a range of low to no response by the antibodies while no significant change in SI-F019.

TABLE 6b

| | | | | | | | |
|---|---|---|---|---|---|---|---|
| WHO Designation | SI-F019 | SI-69C4 | SI-68C5 | SI-69C6 | SI-69C7 | SI-69C8 | SI-69C9 |
| WT | 0.7116 | 1.089 | 1.5033 | 0.7459 | 1.4517 | 0.6397 | 1.6959 |
| alpha | 0.7596 | 1.237 | 1.2967 | 0.7718 | 1.1912 | 0.6967 | 1.3498 |
| delta | 0.6226 | 0.6171 | 1.1999 | 0.7103 | 1.145 | 0.5252 | 1.2973 |
| | | Low | | | | | |
| kappa | 0.8529 | 0.0072 | 1.4715 | 0.9705 | 1.3218 | 0.6285 | 1.5444 |
| | | No | | | | | |
| gamma | 0.7085 | 0.0261 | 1.1662 | 0.1694 | 1.3319 | 0.6571 | 1.5804 |
| | | No | | Low | | | |
| beta | 0.5671 | 0.0212 | 0.9041 | 0.0398 | 1.068 | 0.4734 | 1.1393 |
| | | No | Low | No | Low | Low | Low |
| lambda | 0.9729 | 0.4863 | 1.6376 | 1.1007 | 1.4551 | 0.727 | 1.7596 |
| | | Low | | | | | |

IC50 values for inhibition of viral infectivity in luciferase reporter assay using S protein packaged pseudovirus (NICPBP) to infect 293T cells expressing ACE2. Notably, SI-F019 inhibition of pseudovirus containing variant forms of S protein is more potent than inhibition of pseudovirus containing wild-type S protein based on lower IC50 values.

TABLE 7

| WHO Designation | SARS-CoV-2 Pseudovirus | IC₅₀ (µg/ml) |
|---|---|---|
| Original | WT Wuhan-Hu-1 | 1.311 |
| Alpha | B.1.1.7 | 0.235 |
| Delta | B.1.617.2 | 0.087 |
| Kappa | B.1.617.1 | 0.163 |
| Gamma | P.1 | 0.089 |
| Beta | 501Y.V2 | 0.223 |
| n/a | B.1.617.3 | 0.611 |

TABLE 8a

Demographic summary (age, weight) of the Phase I clinical trial of SI-F019 (NCT04851444)

| | 3 mg/kg | 10 mg/kg | 30 mg/kg | 52 mg/kg | 70 mg/kg | Total |
|---|---|---|---|---|---|---|
| | (n=4) | (n=8) | (n=8) | (n=8) | (n=8) | (n=36) |
| Medium Age | 30.5 | 27.5 | 24.5 | 28 | 27 | 27 |
| (min - max) | (26.0 - 38.0) | (21.0 - 37.0) | (21.0 - 31.0) | (23.0 - 39.0) | (18.0 - 41.0) | (18.0 - 41.0) |
| Medium Weight (kg) | 58.2 | 59.4 | 55.3 | 53.7 | 61.9 | 57.6 |
| (min - max) | (56.5 - 59.5) | (53.0 - 68.1) | (47.7 - 65.4) | (47.3 - 63.9) | (50.8 - 70.8) | (47.3 - 70.8) |

TABLE 8b

Dose escalation and allocation design of the Phase I clinical trial of SI-F019 (NCT04851444)

| | | | | | |
|---|---|---|---|---|---|
| Group | 1 | 2 | 3 | 4 | 5 |
| Dose (mg/kg) | 3 | 10 | 30 | 52 | 70 |
| Dose increment | / | 233% | 200% | 73% | 34% |
| Number of participants (Treatment+ Control) | 2+2 | 6+2 | 6+2 | 6+2 | 6+2 |

TABLE 9a

Summary of all adverse events (AEs) based on system organ class/preferred term (SOC/PT) of the Phase I clinical trial of SI-F019 (NCT04851444)

| Adverse Event | NCI-CTCAE 5.0 AE grade | | | | | |
|---|---|---|---|---|---|---|
| | 3 mg/kg (N*=3) | 10 mg/kg (N=4) | 30 mg/kg (N=4) | 52 mg/kg (N=5) | 70 mg/kg (N=5) | All (N=21) |
| | Grade 1 | Grade 1 | Grade 1 | Grade 1 | Grade 1 | Grade 1 |
| Investigations | 3 (100%) | 3 (75%) | 4 (100%) | 5 (100%) | 5 (100%) | 20 (95%) |
| White blood cell count increased | 1 (33%) | | | | | 1 (5%) |
| White blood cells urine positive | | 1 (25%) | 2 (50%) | 3 (60%) | 2 (40%) | 8 (38%) |
| Urine ketone body present | | 1 (25%) | 1 (25%) | | 1 (20%) | 3 (14%) |
| Glomerular filtration rate decreased | | | 1 (25%) | 1 (20%) | | 2 (10%) |
| Bacterial test positive | 2 (67%) | 2 (50%) | 4 (100%) | 3 (60%) | 3 (60%) | 14 (67%) |
| Electrocardiogram abnormal | | 2 (50%) | | 1 (20%) | | 3 (14%) |
| Blood bilirubin increased | | | 1 (25%) | | | 1 (5%) |
| Blood triglycerides increased | | 1 (25%) | | 1 (20%) | 1 (20%) | 3 (14%) |
| Blood creatinine increased | | | 1 (25%) | 1 (20%) | | 2 (10%) |
| Blood uric acid increased | | | | | 1 (20%) | 1 (5%) |
| Neutrophil count increased | 1 (33%) | | | | | 1 (5%) |
| Renal and urinary disorders | | 1 (25%) | | | | 1 (5%) |
| Haematuria | | 1 (25%) | | | | 1 (5%) |

*N: number of patients who experienced AE(s).

TABLE 9b:

Summary of all treatment related adverse events (TRAEs) based on system organ class/preferred term (SOC/PT) of the Phase I clinical trial of SI-F019 (NCT04851444)

| Adverse Event | NCI-CTCAE 5.0 AE grade | | | | | |
|---|---|---|---|---|---|---|
| | 3 mg/kg (N=2) | 10 mg/kg (N=4) | 30 mg/kg (N=3) | 52 mg/kg (N=4) | 70 mg/kg (N=3) | All (N=16) |
| | Grade 1 | Grade 1 | Grade 1 | Grade 1 | Grade 1 | Grade 1 |
| Investigations | 2 (100%) | 3 (75%) | 3 (100%) | 4 (100%) | 3 (100%) | 15 (94%) |
| White blood cell count increased | 1 (50%) | | | | | 1 (6%) |
| White blood cells urine positive | | 1 (25%) | 1 (33%) | 1 (25%) | | 3 (19%) |
| Urine ketone body present | | 1 (25%) | | | 1 (33%) | 2 (13%) |
| Glomerular filtration rate decreased | | | 1 (33%) | 1 (25%) | | 2 (13%) |
| Bacterial test positive | 1 (50%) | 2 (50%) | 2 (67%) | 1 (25%) | 1 (33%) | 7 (44%) |
| Electrocardiogram abnormal | | 2 (50%) | | 1 (25%) | | 3 (19%) |
| Blood bilirubin increased | | | 1 (33%) | | | 1 (6%) |
| Blood triglycerides increased | | 1 (25%) | | 1 (25%) | 1 (33%) | 3 (19%) |
| Blood creatinine increased | | | 1 (33%) | 1 (25%) | | 2 (13%) |
| Blood uric acid increased | | | | | 1 (33%) | 1 (6%) |
| Neutrophil count increased | 1 (50%) | | | | | 1 (6%) |
| Renal and urinary disorders | | 1 (25%) | | | | 1 (6%) |
| Haematuria | | 1 (25%) | | | | 1 (6%) |

*N: number of patients who experienced TRAE(s).

SEQUENCE LISTING

| Sample ID | Annotation | SEQ ID No. | |
|---|---|---|---|
| | | Protein | DNA |
| huACE2 wild type | huACE2 full length (Genbank_number: Protein: NP_001358344.1; DNA: NM_021804.3) | 1 | 2 |
| huACE2 (1-615) | huACE2 functional domain (residue:1-615) | 3 | 4 |
| | IgG1 Fc wild type | 5 | - |
| | IgG1 Fc null | 6 | - |
| SI-69R2 | A fusion protein of huACE2 (1-615) and IgG1 Fc null | 7 | 8 |
| SI-69R2-G4 | A fusion protein of huACE2 (1-615) and IgG4 Fc | 9 | 10 |
| SI-69R2-A1 | A fusion protein of huACE2 (1-615) and IgA1 Fc | 11 | 12 |
| SI-69R2-A2 | A fusion protein of huACE2 (1-615) and IgA2 Fc | 13 | 14 |
| SI-F019 | A fusion protein of huACE2 (18-615) and IgG1 Fc null (purified from SI-69R2) | 15 | |
| | A fusion protein of huACE2 (18-615) and IgG4 Fc (purified from SI-69R2-G4) | 16 | |
| | A fusion protein of huACE2 (18-615) and IgA1 Fc (purified from SI-69R2-A1) | 17 | |
| | A fusion protein of huACE2 (18-615) and IgA2 Fc (purified from SI-69R2-A2) | 18 | |
| SI-69R3 | A fusion protein of huACE2 (1-615) and IgG1 Fc (w2) | 19 | 20 |
| SI-69R4 | A fusion protein of huACE2 (1-740) and IgG1 Fc (w2) | 21 | 22 |
| SI-69R1 | 6His-tagged huACE2 (1-615) | 23 | 24 |
| SI-69R10 | 6His-tagged human TMPRSS2 protein (residue: 106-492) | 25 | 26 |
| | IgJ chain | 27 | - |
| | Secretory Component | 28 | - |
| SI-69C4 | Bamlanivimab Heavy Chain | 29 | |
| | Bamlanivimab Light Chain | 30 | |
| SI-69C5 | Casirivimab Heavy Chain | 31 | |
| | Casirivimab Light Chain | 32 | |
| SI-69C6 | Etesevimab Heavy Chain | 33 | |
| | Etesevimab Light Chain | 34 | |
| SI-69C7 | Imdevimab Heavy Chain | 35 | |
| | Imdevimab Light Chain | 36 | |
| SI-69C8 | Cilgavimab Heavy Chain | 37 | |
| | Cilgavimab Light Chain | 38 | |
| SI-69C9 | Tixagevimab Heavy Chain | 39 | |
| | Tixagevimab Light Chain | 40 | |

>Sequence ID 1: huACE2 full length protein sequence (Genbank_number:NP_001358344.1, TMPRSS2 protease cutting site)

MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADQSIKVRISLKSALGDKAYEWNDNEMYLFRSSVAYA
MRQYFLKVKNQMILFGEEDVRVANLKPRISFNFFVTAPKNVSDIIPRTEV EKAIRMSRSRINDAFRLNDNSLEFLGIQPTLGPPNQPPVSIWLIVFGVVM
GVIVVGIVILIFTGIRDRKKKNKARSGENPYASIDISKGENNPGFQNTDD
VQTSF

>Sequence ID 2: huACE2 full length DNA sequence (Genbank_number: NM_021804.3)

ATGTCAAGCTCTTCCTGGCTCCTTCTCAGCCTTGTTGCTGTAACTGCTGC
TCAGTCCACCATTGAGGAACAGGCCAAGACATTTTTGGACAAGTTTAACC
ACGAAGCCGAAGACCTGTTCTATCAAAGTTCACTTGCTTCTTGGAATTAT
AACACCAATATTACTGAAGAGAATGTCCAAAACATGAATAATGCTGGGGA
CAAATGGTCTGCCTTTTTAAAGGAACAGTCCACACTTGCCCAAATGTATC
CACTACAAGAAATTCAGAATCTCACAGTCAAGCTTCAGCTGCAGGCTCTT
CAGCAAAATGGGTCTTCAGTGCTCTCAGAAGACAAGAGCAAACGGTTGAA
CACAATTCTAAATACAATGAGCACCATCTACAGTACTGGAAAAGTTTGTA
ACCCAGATAATCCACAAGAATGCTTATTACTTGAACCAGGTTTGAATGAA
ATAATGGCAAACAGTTTAGACTACAATGAGAGGCTCTGGGCTTGGGAAAG
CTGGAGATCTGAGGTCGGCAAGCAGCTGAGGCCATTATATGAAGAGTATG
TGGTCTTGAAAAATGAGATGGCAAGAGCAAATCATTATGAGGACTATGGG
GATTATTGGAGAGGAGACTATGAAGTAAATGGGGTAGATGGCTATGACTA
CAGCCGCGGCCAGTTGATTGAAGATGTGGAACATACCTTTGAAGAGATTA
AACCATTATATGAACATCTTCATGCCTATGTGAGGGCAAAGTTGATGAAT
GCCTATCCTTCCTATATCAGTCCAATTGGATGCCTCCCTGCTCATTTGCT
TGGTGATATGTGGGGTAGATTTTGGACAAATCTGTACTCTTTGACAGTTC
CCTTTGGACAGAAACCAAACATAGATGTTACTGATGCAATGGTGGACCAG
GCCTGGGATGCACAGAGAATATTCAAGGAGGCCGAGAAGTTCTTTGTATC
TGTTGGTCTTCCTAATATGACTCAAGGATTCTGGGAAAATTCCATGCTAA
CGGACCCAGGAAATGTTCAGAAAGCAGTCTGCCATCCCACAGCTTGGGAC
CTGGGGAAGGGCGACTTCAGGATCCTTATGTGCACAAAGGTGACAATGGA
CGACTTCCTGACAGCTCATCATGAGATGGGGCATATCCAGTATGATATGG
CATATGCTGCACAACCTTTTCTGCTAAGAAATGGAGCTAATGAAGGATTC
CATGAAGCTGTTGGGGAAATCATGTCACTTTCTGCAGCCACACCTAAGCA
TTTAAAATCCATTGGTCTTCTGTCACCCGATTTTCAAGAAGACAATGAAA
CAGAAATAAACTTCCTGCTCAAACAAGCACTCACGATTGTTGGGACTCTG
CCATTTACTTACATGTTAGAGAAGTGGAGGTGGATGGTCTTTAAAGGGGA
AATTCCCAAAGACCAGTGGATGAAAAAGTGGTGGGAGATGAAGCGAGAGA
TAGTTGGGGTGGTGGAACCTGTGCCCCATGATGAAACATACTGTGACCCC
GCATCTCTGTTCCATGTTTCTAATGATTACTCATTCATTCGATATTACAC
AAGGACCCTTTACCAATTCCAGTTTCAAGAAGCACTTTGTCAAGCAGCTA
AACATGAAGGCCCTCTGCACAAATGTGACATCTCAAACTCTACAGAAGCT
GGACAGAAACTGTTCAATATGCTGAGGCTTGGAAAATCAGAACCCTGGAC
CCTAGCATTGGAAAATGTTGTAGGAGCAAAGAACATGAATGTAAGGCCAC
TGCTCAACTACTTTGAGCCCTTATTTACCTGGCTGAAAGACCAGAACAAG
AATTCTTTTGTGGGATGGAGTACCGACTGGAGTCCATATGCAGACCAAAG
CATCAAAGTGAGGATAAGCCTAAAATCAGCTCTTGGAGATAAAGCATATG
AATGGAACGACAATGAAATGTACCTGTTCCGATCATCTGTTGCATATGCT
ATGAGGCAGTACTTTTTAAAAGTAAAAAATCAGATGATTCTTTTTGGGGA
GGAGGATGTGCGAGTGGCTAATTTGAAACCAAGAATCTCCTTTAATTTCT
TTGTCACTGCACCTAAAAATGTGTCTGATATCATTCCTAGAACTGAAGTT
GAAAAGGCCATCAGGATGTCCCGGAGCCGTATCAATGATGCTTTCCGTCT
GAATGACAACAGCCTAGAGTTTCTGGGGATACAGCCAACACTTGGACCTC
CTAACCAGCCCCCTGTTTCCATATGGCTGATTGTTTTTGGAGTTGTGATG
GGAGTGATAGTGGTTGGCATTGTCATCCTGATCTTCACTGGGATCAGAGA
TCGGAAGAAGAAAAATAAAGCAAGAAGTGGAGAAAATCCTTATGCCTCCA
TCGATATTAGCAAAGGAGAAAATAATCCAGGATTCCAAAACACTGATGAT
GTTCAGACCTCCTTTTAG

>Sequence ID 3: huACE2 functional domain (residue:1-615) protein sequence

MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYAD

>Sequence ID 4: huACE2 functional domain (residue:1-615) DNA sequence

ATGTCAAGCTCTTCCTGGCTCCTTCTCAGCCTTGTTGCTGTAACTGCTGC
TCAGTCCACCATTGAGGAACAGGCCAAGACATTTTTGGACAAGTTTAACC
ACGAAGCCGAAGACCTGTTCTATCAAAGTTCACTTGCTTCTTGGAATTAT
AACACCAATATTACTGAAGAGAATGTCCAAAACATGAATAATGCTGGGGA
CAAATGGTCTGCCTTTTTAAAGGAACAGTCCACACTTGCCCAAATGTATC
CACTACAAGAAATTCAGAATCTCACAGTCAAGCTTCAGCTGCAGGCTCTT
CAGCAAAATGGGTCTTCAGTGCTCTCAGAAGACAAGAGCAAACGGTTGAA
CACAATTCTAAATACAATGAGCACCATCTACAGTACTGGAAAAGTTTGTA
ACCCAGATAATCCACAAGAATGCTTATTACTTGAACCAGGTTTGAATGAA
ATAATGGCAAACAGTTTAGACTACAATGAGAGGCTCTGGGCTTGGGAAAG
CTGGAGATCTGAGGTCGGCAAGCAGCTGAGGCCATTATATGAAGAGTATG
TGGTCTTGAAAAATGAGATGGCAAGAGCAAATCATTATGAGGACTATGGG
GATTATTGGAGAGGAGACTATGAAGTAAATGGGGTAGATGGCTATGACTA
CAGCCGCGGCCAGTTGATTGAAGATGTGGAACATACCTTTGAAGAGATTA
AACCATTATATGAACATCTTCATGCCTATGTGAGGGCAAAGTTGATGAAT
GCCTATCCTTCCTATATCAGTCCAATTGGATGCCTCCCTGCTCATTTGCT
TGGTGATATGTGGGGTAGATTTTGGACAAATCTGTACTCTTTGACAGTTC
CCTTTGGACAGAAACCAAACATAGATGTTACTGATGCAATGGTGGACCAG
GCCTGGGATGCACAGAGAATATTCAAGGAGGCCGAGAAGTTCTTTGTATC
TGTTGGTCTTCCTAATATGACTCAAGGATTCTGGGAAAATTCCATGCTAA
CGGACCCAGGAAATGTTCAGAAAGCAGTCTGCCATCCCACAGCTTGGGAC
CTGGGGAAGGGCGACTTCAGGATCCTTATGTGCACAAAGGTGACAATGGA
CGACTTCCTGACAGCTCATCATGAGATGGGGCATATCCAGTATGATATGG
CATATGCTGCACAACCTTTTCTGCTAAGAAATGGAGCTAATGAAGGATTC
CATGAAGCTGTTGGGGAAATCATGTCACTTTCTGCAGCCACACCTAAGCA
TTTAAAATCCATTGGTCTTCTGTCACCCGATTTTCAAGAAGACAATGAAA
CAGAAATAAACTTCCTGCTCAAACAAGCACTCACGATTGTTGGGACTCTG
CCATTTACTTACATGTTAGAGAAGTGGAGGTGGATGGTCTTTAAAGGGGA
AATTCCCAAAGACCAGTGGATGAAAAAGTGGTGGGAGATGAAGCGAGAGA
TAGTTGGGGTGGTGGAACCTGTGCCCCATGATGAAACATACTGTGACCCC
GCATCTCTGTTCCATGTTTCTAATGATTACTCATTCATTCGATATTACAC
AAGGACCCTTTACCAATTCCAGTTTCAAGAAGCACTTTGTCAAGCAGCTA
AACATGAAGGCCCTCTGCACAAATGTGACATCTCAAACTCTACAGAAGCT
GGACAGAAACTGTTCAATATGCTGAGGCTTGGAAAATCAGAACCCTGGAC
CCTAGCATTGGAAAATGTTGTAGGAGCAAAGAACATGAATGTAAGGCCAC
TGCTCAACTACTTTGAGCCCTTATTTACCTGGCTGAAAGACCAGAACAAG
AATTCTTTTGTGGGATGGAGTACCGACTGGAGTCCATATGCAGAC

>Sequence ID 5: Fc wild type IgG1 Fc (EU numbering 216-447)

EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK

>Sequence ID 6: Fc null version (EU numbering 216-447, with mutations: C220S, L234A, L235A, and K322A)

EPKSSDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLN
GKEYKCAVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK

>Sequence ID 7: SI-69R2_huACE2 functional domain (residue:1-615)- IgG1 Fc (null) protein sequence (EU numbering 216-447, with mutations: C220S, L234A, L235A, and K322A)

MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADEPKSSDKTHTCPPCPAPEAAGGPSVFLFPPKPKDT
LMISRTPEVTCVVVDVSHEDPEVKFNWYVDGVEVHNAKTKPREEQYNSTY
RVVSVLTVLHQDWLNGKEYKCAVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK

>Sequence ID 8:SI-69R2: huACE2 functional domain (residue:1-615)- IgG1 Fc (null) DNA sequence

ATGTCAAGCTCTTCCTGGCTCCTTCTCAGCCTTGTTGCTGTAACTGCTGC
TCAGTCCACCATTGAGGAACAGGCCAAGACATTTTTGGACAAGTTTAACC
ACGAAGCCGAAGACCTGTTCTATCAAAGTTCACTTGCTTCTTGGAATTAT
AACACCAATATTACTGAAGAGAATGTCCAAAACATGAATAATGCTGGGGA
CAAATGGTCTGCCTTTTTAAAGGAACAGTCCACACTTGCCCAAATGTATC
CACTACAAGAAATTCAGAATCTCACAGTCAAGCTTCAGCTGCAGGCTCTT
CAGCAAAATGGGTCTTCAGTGCTCTCAGAAGACAAGAGCAAACGGTTGAA
CACAATTCTAAATACAATGAGCACCATCTACAGTACTGGAAAAGTTTGTA
ACCCAGATAATCCACAAGAATGCTTATTACTTGAACCAGGTTTGAATGAA
ATAATGGCAAACAGTTTAGACTACAATGAGAGGCTCTGGGCTTGGGAAAG
CTGGAGATCTGAGGTCGGCAAGCAGCTGAGGCCATTATATGAAGAGTATG
TGGTCTTGAAAAATGAGATGGCAAGAGCAAATCATTATGAGGACTATGGG
GATTATTGGAGAGGAGACTATGAAGTAAATGGGGTAGATGGCTATGACTA
CAGCCGCGGCCAGTTGATTGAAGATGTGGAACATACCTTTGAAGAGATTA
AACCATTATATGAACATCTTCATGCCTATGTGAGGGCAAAGTTGATGAAT
GCCTATCCTTCCTATATCAGTCCAATTGGATGCCTCCCTGCTCATTTGCT
TGGTGATATGTGGGGTAGATTTTGGACAAATCTGTACTCTTTGACAGTTC
CCTTTGGACAGAAACCAAACATAGATGTTACTGATGCAATGGTGGACCAG
GCCTGGGATGCACAGAGAATATTCAAGGAGGCCGAGAAGTTCTTTGTATC
TGTTGGTCTTCCTAATATGACTCAAGGATTCTGGGAAAATTCCATGCTAA
CGGACCCAGGAAATGTTCAGAAAGCAGTCTGCCATCCCACAGCTTGGGAC
CTGGGGAAGGGCGACTTCAGGATCCTTATGTGCACAAAGGTGACAATGGA
CGACTTCCTGACAGCTCATCATGAGATGGGGCATATCCAGTATGATATGG
CATATGCTGCACAACCTTTTCTGCTAAGAAATGGAGCTAATGAAGGATTC
CATGAAGCTGTTGGGGAAATCATGTCACTTTCTGCAGCCACACCTAAGCA
TTTAAAATCCATTGGTCTTCTGTCACCCGATTTTCAAGAAGACAATGAAA
CAGAAATAAACTTCCTGCTCAAACAAGCACTCACGATTGTTGGGACTCTG
CCATTTACTTACATGTTAGAGAAGTGGAGGTGGATGGTCTTTAAAGGGGA
AATTCCCAAAGACCAGTGGATGAAAAAGTGGTGGGAGATGAAGCGAGAGA
TAGTTGGGGTGGTGGAACCTGTGCCCCATGATGAAACATACTGTGACCCC
GCATCTCTGTTCCATGTTTCTAATGATTACTCATTCATTCGATATTACAC
AAGGACCCTTTACCAATTCCAGTTTCAAGAAGCACTTTGTCAAGCAGCTA
AACATGAAGGCCCTCTGCACAAATGTGACATCTCAAACTCTACAGAAGCT
GGACAGAAACTGTTCAATATGCTGAGGCTTGGAAAATCAGAACCCTGGAC
CCTAGCATTGGAAAATGTTGTAGGAGCAAAGAACATGAATGTAAGGCCAC
TGCTCAACTACTTTGAGCCCTTATTTACCTGGCTGAAAGACCAGAACAAG
AATTCTTTTGTGGGATGGAGTACCGACTGGAGTCCATATGCAGACGAGCC
CAAATCTTCCGACAAAACTCACACATGCCCACCGTGCCCAGCACCTGAAG
CCGCGGGGGGACCGTCAGTCTTCCTCTTCCCCCCAAAACCCAAGGACACC
CTCATGATCTCCCGGACCCCTGAGGTCACATGCGTGGTGGTGGACGTGAG
CCACGAAGACCCTGAGGTCAAGTTCAACTGGTACGTGGACGGCGTGGAGG
TGCATAATGCCAAGACAAAGCCGCGGGAGGAGCAGTACAACAGCACGTAC
CGTGTGGTCAGCGTCCTCACCGTCCTGCACCAGGACTGGCTGAATGGCAA
GGAGTACAAGTGCGCGGTCTCCAACAAAGCCCTCCCAGCCCCCATCGAGA
AAACCATCTCCAAAGCCAAAGGGCAGCCCCGAGAACCACAGGTGTACACC
CTGCCCCCATCCCGGGATGAGCTGACCAAGAACCAGGTCAGCCTGACCTG
CCTGGTCAAAGGCTTCTATCCCAGCGACATCGCCGTGGAGTGGGAGAGCA
ATGGGCAGCCGGAGAACAACTACAAGACCACGCCTCCCGTGCTGGACTCC
GACGGCTCCTTCTTCCTCTATAGCAAGCTCACCGTGGACAAGAGCAGGTG
GCAGCAGGGGAACGTCTTCTCATGCTCCGTGATGCATGAGGCTCTGCACA
ACCACTACACGCAGAAGAGCCTCTCCCTGTCTCCGGGTAAATAG

>Sequence ID 9: huACE2 functional domain (residue:1-615)- IgG4 Fc protein sequence

MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADESKYGPPCPPCPAPEFLGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK

>Sequence ID 10: huACE2 functional domain (residue:1-615)- IgG4 Fc DNA sequence

ATGTCAAGCTCTTCCTGGCTCCTTCTCAGCCTTGTTGCTGTAACTGCTGC
TCAGTCCACCATTGAGGAACAGGCCAAGACATTTTTGGACAAGTTTAACC
ACGAAGCCGAAGACCTGTTCTATCAAAGTTCACTTGCTTCTTGGAATTAT
AACACCAATATTACTGAAGAGAATGTCCAAAACATGAATAATGCTGGGGA
CAAATGGTCTGCCTTTTTAAAGGAACAGTCCACACTTGCCCAAATGTATC
CACTACAAGAAATTCAGAATCTCACAGTCAAGCTTCAGCTGCAGGCTCTT
CAGCAAAATGGGTCTTCAGTGCTCTCAGAAGACAAGAGCAAACGGTTGAA
CACAATTCTAAATACAATGAGCACCATCTACAGTACTGGAAAAGTTTGTA
ACCCAGATAATCCACAAGAATGCTTATTACTTGAACCAGGTTTGAATGAA
ATAATGGCAAACAGTTTAGACTACAATGAGAGGCTCTGGGCTTGGGAAAG
CTGGAGATCTGAGGTCGGCAAGCAGCTGAGGCCATTATATGAAGAGTATG
TGGTCTTGAAAAATGAGATGGCAAGAGCAAATCATTATGAGGACTATGGG
GATTATTGGAGAGGAGACTATGAAGTAAATGGGGTAGATGGCTATGACTA
CAGCCGCGGCCAGTTGATTGAAGATGTGGAACATACCTTTGAAGAGATTA
AACCATTATATGAACATCTTCATGCCTATGTGAGGGCAAAGTTGATGAAT
GCCTATCCTTCCTATATCAGTCCAATTGGATGCCTCCCTGCTCATTTGCT
TGGTGATATGTGGGGTAGATTTTGGACAAATCTGTACTCTTTGACAGTTC
CCTTTGGACAGAAACCAAACATAGATGTTACTGATGCAATGGTGGACCAG
GCCTGGGATGCACAGAGAATATTCAAGGAGGCCGAGAAGTTCTTTGTATC
TGTTGGTCTTCCTAATATGACTCAAGGATTCTGGGAAAATTCCATGCTAA
CGGACCCAGGAAATGTTCAGAAAGCAGTCTGCCATCCCACAGCTTGGGAC
CTGGGGAAGGGCGACTTCAGGATCCTTATGTGCACAAAGGTGACAATGGA
CGACTTCCTGACAGCTCATCATGAGATGGGGCATATCCAGTATGATATGG
CATATGCTGCACAACCTTTTCTGCTAAGAAATGGAGCTAATGAAGGATTC
CATGAAGCTGTTGGGGAAATCATGTCACTTTCTGCAGCCACACCTAAGCA
TTTAAAATCCATTGGTCTTCTGTCACCCGATTTTCAAGAAGACAATGAAA
CAGAAATAAACTTCCTGCTCAAACAAGCACTCACGATTGTTGGGACTCTG
CCATTTACTTACATGTTAGAGAAGTGGAGGTGGATGGTCTTTAAAGGGGA
AATTCCCAAAGACCAGTGGATGAAAAAGTGGTGGGAGATGAAGCGAGAGA
TAGTTGGGGTGGTGGAACCTGTGCCCCATGATGAAACATACTGTGACCCC
GCATCTCTGTTCCATGTTTCTAATGATTACTCATTCATTCGATATTACAC
AAGGACCCTTTACCAATTCCAGTTTCAAGAAGCACTTTGTCAAGCAGCTA
AACATGAAGGCCCTCTGCACAAATGTGACATCTCAAACTCTACAGAAGCT
GGACAGAAACTGTTCAATATGCTGAGGCTTGGAAAATCAGAACCCTGGAC
CCTAGCATTGGAAAATGTTGTAGGAGCAAAGAACATGAATGTAAGGCCAC
TGCTCAACTACTTTGAGCCCTTATTTACCTGGCTGAAAGACCAGAACAAG
AATTCTTTTGTGGGATGGAGTACCGACTGGAGTCCATATGCAGACGAGTC
CAAATATGGTCCCCCGTGCCCACCATGCCCAGCACCTGAGTTCCTGGGGG
GACCATCAGTCTTCCTGTTCCCCCCAAAACCCAAGGACACTCTCATGATC
TCCCGGACCCCTGAGGTCACGTGCGTGGTGGTGGACGTGAGCCAGGAAGA
CCCCGAGGTCCAGTTCAACTGGTACGTGGATGGCGTGGAGGTGCATAATG
CCAAGACAAAGCCGCGGGAGGAGCAGTTCAACAGCACGTACCGTGTGGTC
AGCGTCCTCACCGTCCTCCACCAGGACTGGCTGAACGGCAAGGAGTACAA
GTGCAAGGTCTCCAACAAAGGCCTCCCGTCCTCCATCGAGAAAACCATCT
CCAAAGCCAAAGGGCAGCCCCGAGAGCCACAGGTGTACACCCTGCCCCCA
TCCCAGGAGGAGATGACCAAGAACCAGGTCAGCCTGACCTGCCTGGTCAA
AGGCTTCTACCCCAGCGACATCGCCGTGGAGTGGGAGAGCAATGGGCAGC
CGGAGAACAACTACAAGACCACGCCTCCCGTGCTGGACTCCGACGGCTCC
TTCTTCCTCTACAGCAGGCTAACCGTGGACAAGAGCAGGTGGCAGGAGGG
GAATGTCTTCTCATGCTCCGTGATGCATGAGGCTCTGCACAACCACTACA
CGCAGAAGAGCCTCTCCCTGTCTCTGGGTAAATGA

>Sequence ID 11: huACE2 functional domain (residue:1-615)- IgA1 Fc Protein sequence

MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADSQDVTVPCPVPSTPPTPSPSTPPTPSPSCCHPRLS
LHRPALEDLLLGSEANLTCTLTGLRDASGVTFTWTPSSGKSAVQGPPERD
LCGCYSVSSVLPGCAEPWNHGKTFTCTAAYPESKTPLTATLSKSGNTFRP
EVHLLPPPSEELALNELVTLTCLARGFSPKDVLVRWLQGSQELPREKYLT
WASRQEPSQGTTTFAVTSILRVAAEDWKKGDTFSCMVGHEALPLAFTQKT
IDRLAGKPTHVNVSVVMAEVDGTCY

>Sequence ID 12: huACE2 functional domain (residue:1-615)- IgA1 Fc DNA sequence

ATGTCAAGCTCTTCCTGGCTCCTTCTCAGCCTTGTTGCTGTAACTGCTGC
TCAGTCCACCATTGAGGAACAGGCCAAGACATTTTTGGACAAGTTTAACC
ACGAAGCCGAAGACCTGTTCTATCAAAGTTCACTTGCTTCTTGGAATTAT
AACACCAATATTACTGAAGAGAATGTCCAAAACATGAATAATGCTGGGGA
CAAATGGTCTGCCTTTTTAAAGGAACAGTCCACACTTGCCCAAATGTATC
CACTACAAGAAATTCAGAATCTCACAGTCAAGCTTCAGCTGCAGGCTCTT
CAGCAAAATGGGTCTTCAGTGCTCTCAGAAGACAAGAGCAAACGGTTGAA
CACAATTCTAAATACAATGAGCACCATCTACAGTACTGGAAAAGTTTGTA
ACCCAGATAATCCACAAGAATGCTTATTACTTGAACCAGGTTTGAATGAA
ATAATGGCAAACAGTTTAGACTACAATGAGAGGCTCTGGGCTTGGGAAAG
CTGGAGATCTGAGGTCGGCAAGCAGCTGAGGCCATTATATGAAGAGTATG
TGGTCTTGAAAAATGAGATGGCAAGAGCAAATCATTATGAGGACTATGGG
GATTATTGGAGAGGAGACTATGAAGTAAATGGGGTAGATGGCTATGACTA
CAGCCGCGGCCAGTTGATTGAAGATGTGGAACATACCTTTGAAGAGATTA
AACCATTATATGAACATCTTCATGCCTATGTGAGGGCAAAGTTGATGAAT
GCCTATCCTTCCTATATCAGTCCAATTGGATGCCTCCCTGCTCATTTGCT
TGGTGATATGTGGGGTAGATTTTGGACAAATCTGTACTCTTTGACAGTTC
CCTTTGGACAGAAACCAAACATAGATGTTACTGATGCAATGGTGGACCAG
GCCTGGGATGCACAGAGAATATTCAAGGAGGCCGAGAAGTTCTTTGTATC
TGTTGGTCTTCCTAATATGACTCAAGGATTCTGGGAAAATTCCATGCTAA
CGGACCCAGGAAATGTTCAGAAAGCAGTCTGCCATCCCACAGCTTGGGAC
CTGGGGAAGGGCGACTTCAGGATCCTTATGTGCACAAAGGTGACAATGGA
CGACTTCCTGACAGCTCATCATGAGATGGGGCATATCCAGTATGATATGG
CATATGCTGCACAACCTTTTCTGCTAAGAAATGGAGCTAATGAAGGATTC
CATGAAGCTGTTGGGGAAATCATGTCACTTTCTGCAGCCACACCTAAGCA
TTTAAAATCCATTGGTCTTCTGTCACCCGATTTTCAAGAAGACAATGAAA
CAGAAATAAACTTCCTGCTCAAACAAGCACTCACGATTGTTGGGACTCTG
CCATTTACTTACATGTTAGAGAAGTGGAGGTGGATGGTCTTTAAAGGGGA
AATTCCCAAAGACCAGTGGATGAAAAAGTGGTGGGAGATGAAGCGAGAGA
TAGTTGGGGTGGTGGAACCTGTGCCCCATGATGAAACATACTGTGACCCC
GCATCTCTGTTCCATGTTTCTAATGATTACTCATTCATTCGATATTACAC
AAGGACCCTTTACCAATTCCAGTTTCAAGAAGCACTTTGTCAAGCAGCTA
AACATGAAGGCCCTCTGCACAAATGTGACATCTCAAACTCTACAGAAGCT
GGACAGAAACTGTTCAATATGCTGAGGCTTGGAAAATCAGAACCCTGGAC
CCTAGCATTGGAAAATGTTGTAGGAGCAAAGAACATGAATGTAAGGCCAC
TGCTCAACTACTTTGAGCCCTTATTTACCTGGCTGAAAGACCAGAACAAG
AATTCTTTTGTGGGATGGAGTACCGACTGGAGTCCATATGCAGACAGCCA
GGATGTGACTGTGCCCTGCCCAGTTCCCTCAACTCCACCTACCCCATCTC
CCTCAACTCCACCTACCCCATCTCCCTCATGCTGCCACCCCCGACTGTCA
CTGCACCGACCGGCCCTCGAGGACCTGCTCTTAGGTTCAGAAGCGAACCT
CACGTGCACACTGACCGGCCTGAGAGATGCCTCAGGTGTCACCTTCACCT
GGACGCCCTCAAGTGGGAAGAGCGCTGTTCAAGGACCACCTGAGCGTGAC
CTCTGTGGCTGCTACAGCGTGTCCAGTGTCCTGCCGGGCTGTGCCGAGCC
ATGGAACCATGGGAAGACCTTCACTTGCACTGCTGCCTACCCCGAGTCCA
AGACCCCGCTAACCGCCACCCTCTCAAAATCCGGAAACACATTCCGGCCC
GAGGTCCACCTGCTGCCGCCGCCGTCGGAGGAGCTGGCCCTGAACGAGCT
GGTGACGCTGACGTGCCTGGCACGCGGCTTCAGCCCCAAGGACGTGCTGG
TTCGCTGGCTGCAGGGGTCACAGGAGCTGCCCCGCGAGAAGTACCTGACT
TGGGCATCCCGGCAGGAGCCCAGCCAGGGCACCACCACCTTCGCTGTGAC
CAGCATACTGCGCGTGGCAGCCGAGGACTGGAAGAAGGGGGACACCTTCT
CCTGCATGGTGGGCCACGAGGCCCTGCCGCTGGCCTTCACACAGAAGACC
ATCGACCGCTTGGCGGGTAAACCCACCCATGTCAATGTGTCTGTTGTCAT
GGCGGAGGTGGACGGCACCTGCTACTGA

>Sequence ID 13: huACE2 functional domain (residue:1-615)- IgA2 Fc Protein sequence

MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADSQDVTVPCRVPPPPPCCHPRLSLHRPALEDLLLGS
EANLTCTLTGLRDASGATFTWTPSSGKSAVQGPPERDLCGCYSVSSVLPG
CAQPWNHGETFTCTAAHPELKTPLTANITKSGNTFRPEVHLLPPPSEELA
LNELVTLTCLARGFSPKDVLVRWLQGSQELPREKYLTWASRQEPSQGTTT
YAVTSILRVAAEDWKKGETFSCMVGHEALPLAFTQKTIDRMAGKPTHINV
SVVMAEADGTCY

>Sequence ID 14: huACE2 functional domain (residue:1-615)- IgA2 Fc DNA sequence

ATGTCAAGCTCTTCCTGGCTCCTTCTCAGCCTTGTTGCTGTAACTGCTGC
TCAGTCCACCATTGAGGAACAGGCCAAGACATTTTTGGACAAGTTTAACC
ACGAAGCCGAAGACCTGTTCTATCAAAGTTCACTTGCTTCTTGGAATTAT
AACACCAATATTACTGAAGAGAATGTCCAAAACATGAATAATGCTGGGGA
CAAATGGTCTGCCTTTTTAAAGGAACAGTCCACACTTGCCCAAATGTATC
CACTACAAGAAATTCAGAATCTCACAGTCAAGCTTCAGCTGCAGGCTCTT
CAGCAAAATGGGTCTTCAGTGCTCTCAGAAGACAAGAGCAAACGGTTGAA
CACAATTCTAAATACAATGAGCACCATCTACAGTACTGGAAAAGTTTGTA
ACCCAGATAATCCACAAGAATGCTTATTACTTGAACCAGGTTTGAATGAA
ATAATGGCAAACAGTTTAGACTACAATGAGAGGCTCTGGGCTTGGGAAAG
CTGGAGATCTGAGGTCGGCAAGCAGCTGAGGCCATTATATGAAGAGTATG
TGGTCTTGAAAAATGAGATGGCAAGAGCAAATCATTATGAGGACTATGGG
GATTATTGGAGAGGAGACTATGAAGTAAATGGGGTAGATGGCTATGACTA
CAGCCGCGGCCAGTTGATTGAAGATGTGGAACATACCTTTGAAGAGATTA
AACCATTATATGAACATCTTCATGCCTATGTGAGGGCAAAGTTGATGAAT
GCCTATCCTTCCTATATCAGTCCAATTGGATGCCTCCCTGCTCATTTGCT
TGGTGATATGTGGGGTAGATTTTGGACAAATCTGTACTCTTTGACAGTTC
CCTTTGGACAGAAACCAAACATAGATGTTACTGATGCAATGGTGGACCAG
GCCTGGGATGCACAGAGAATATTCAAGGAGGCCGAGAAGTTCTTTGTATC
TGTTGGTCTTCCTAATATGACTCAAGGATTCTGGGAAAATTCCATGCTAA
CGGACCCAGGAAATGTTCAGAAAGCAGTCTGCCATCCCACAGCTTGGGAC
CTGGGGAAGGGCGACTTCAGGATCCTTATGTGCACAAAGGTGACAATGGA
CGACTTCCTGACAGCTCATCATGAGATGGGGCATATCCAGTATGATATGG
CATATGCTGCACAACCTTTTCTGCTAAGAAATGGAGCTAATGAAGGATTC
CATGAAGCTGTTGGGGAAATCATGTCACTTTCTGCAGCCACACCTAAGCA
TTTAAAATCCATTGGTCTTCTGTCACCCGATTTTCAAGAAGACAATGAAA
CAGAAATAAACTTCCTGCTCAAACAAGCACTCACGATTGTTGGGACTCTG
CCATTTACTTACATGTTAGAGAAGTGGAGGTGGATGGTCTTTAAAGGGGA
AATTCCCAAAGACCAGTGGATGAAAAAGTGGTGGGAGATGAAGCGAGAGA
TAGTTGGGGTGGTGGAACCTGTGCCCCATGATGAAACATACTGTGACCCC
GCATCTCTGTTCCATGTTTCTAATGATTACTCATTCATTCGATATTACAC
AAGGACCCTTTACCAATTCCAGTTTCAAGAAGCACTTTGTCAAGCAGCTA
AACATGAAGGCCCTCTGCACAAATGTGACATCTCAAACTCTACAGAAGCT
GGACAGAAACTGTTCAATATGCTGAGGCTTGGAAAATCAGAACCCTGGAC
CCTAGCATTGGAAAATGTTGTAGGAGCAAAGAACATGAATGTAAGGCCAC
TGCTCAACTACTTTGAGCCCTTATTTACCTGGCTGAAAGACCAGAACAAG
AATTCTTTTGTGGGATGGAGTACCGACTGGAGTCCATATGCAGACAGCCA
GGATGTGACTGTGCCCTGCCGAGTTCCCCCACCTCCCCCATGCTGCCACC
CCCGACTGTCGCTGCACCGACCGGCCCTCGAGGACCTGCTCTTAGGTTCA
GAAGCGAACCTCACGTGCACACTGACCGGCCTGAGAGATGCCTCTGGTGC
CACCTTCACCTGGACGCCCTCAAGTGGGAAGAGCGCTGTTCAAGGACCAC
CTGAGCGTGACCTCTGTGGCTGCTACAGCGTGTCCAGTGTCCTGCCTGGC
TGTGCCCAGCCATGGAACCATGGGGAGACCTTCACCTGCACTGCTGCCCA
CCCCGAGTTGAAGACCCCACTAACCGCCAACATCACAAAATCCGGAAACA
CATTCCGGCCCGAGGTCCACCTGCTGCCGCCGCCGTCGGAGGAGCTGGCC
CTGAACGAGCTGGTGACGCTGACGTGCCTGGCACGTGGCTTCAGCCCCAA
GGATGTGCTGGTTCGCTGGCTGCAGGGGTCACAGGAGCTGCCCCGCGAGA
AGTACCTGACTTGGGCATCCCGGCAGGAGCCCAGCCAGGGCACCACCACC
TATGCTGTGACCAGCATACTGCGCGTGGCAGCCGAGGACTGGAAGAAGGG
GGAAACCTTCTCCTGCATGGTGGGCCACGAGGCCCTGCCGCTGGCCTTCA
CACAGAAGACCATCGACCGCATGGCGGGTAAACCCACCCATATCAATGTG
TCTGTTGTCATGGCGGAGGCGGACGGCACCTGCTACTGA

>Sequence ID 15: SI-F019_huACE2 functional domain (residue:18-615)- IgG1 Fc (null) protein sequence (with mutations at C220S, L234A, L235A, and K322A, EU numbering)

QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADEP
KSSDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCAVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

>Sequence ID 16: huACE2 functional domain (residue:18-615)- IgG4 Fc protein sequence

QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGREWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK

>Sequence ID 17: huACE2 functional domain (residue:18-615)- IgA1 Fc Protein sequence

QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGREWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADSQ
DVTVPCPVPSTPPTPSPSTPPTPSPSCCHPRLSLHRPALEDLLLGSEANL
TCTLTGLRDASGVTFTWTPSSGKSAVQGPPERDLCGCYSVSSVLPGCAEP
WNHGKTFTCTAAYPESKTPLTATLSKSGNTFRPEVHLLPPPSEELALNEL
VTLTCLARGFSPKDVLVRWLQGSQELPREKYLTWASRQEPSQGTTTFAVT
SILRVAAEDWKKGDTFSCMVGHEALPLAFTQKTIDRLAGKPTHVNVSVVM
AEVDGTCY

>Sequence ID 18: huACE2 functional domain (residue:18-615)- IgA2 Fc Protein sequence

QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGREWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADSQ
DVTVPCRVPPPPPCCHPRLSLHRPALEDLLLGSEANLTCTLTGLRDASGA
TFTWTPSSGKSAVQGPPERDLCGCYSVSSVLPGCAQPWNHGETFTCTAAH
PELKTPLTANITKSGNTFRPEVHLLPPPSEELALNELVTLTCLARGFSPK
DVLVRWLQGSQELPREKYLTWASRQEPSQGTTTYAVTSILRVAAEDWKKG
ETFSCMVGHEALPLAFTQKTIDRMAGKPTHINVSVVMAEADGTCY

>Sequence ID 19: SI-69R3_human ACE2-ECD-1-615-Fc-w2 (EU numbering 216-447)-protein sequence

MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADEPKSSDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK

>Sequence ID 20: SI-69R3_human ACE2-ECD-1-615-Fc-w2-DNA sequence

ATGTCAAGCTCTTCCTGGCTCCTTCTCAGCCTTGTTGCTGTAACTGCTGC
TCAGTCCACCATTGAGGAACAGGCCAAGACATTTTTGGACAAGTTTAACC
ACGAAGCCGAAGACCTGTTCTATCAAAGTTCACTTGCTTCTTGGAATTAT
AACACCAATATTACTGAAGAGAATGTCCAAAACATGAATAATGCTGGGGA
CAAATGGTCTGCCTTTTTAAAGGAACAGTCCACACTTGCCCAAATGTATC
CACTACAAGAAATTCAGAATCTCACAGTCAAGCTCCAGCTGCAGGCTCTT
CAGCAAAATGGGTCTTCAGTGCTCTCAGAAGACAAGAGCAAACGGTTGAA
CACAATTCTAAATACAATGAGCACCATCTACAGTACTGGAAAAGTTTGTA
ACCCAGATAATCCACAAGAATGCTTATTACTTGAACCAGGTTTGAATGAA
ATAATGGCAAACAGTTTAGACTACAATGAGAGGCTCTGGGCTTGGGAAAG
CTGGAGATCTGAGGTCGGCAAGCAGCTGAGGCCATTATATGAAGAGTATG
TGGTCTTGAAAAATGAGATGGCAAGAGCAAATCATTATGAGGACTATGGG
GATTATTGGAGAGGAGACTATGAAGTAAATGGGGTAGATGGCTATGACTA
CAGCCGCGGCCAGTTGATTGAAGATGTGGAACATACCTTTGAAGAGATTA
AACCATTATATGAACATCTTCATGCCTATGTGAGGGCAAAGTTGATGAAT
GCCTATCCTTCCTATATCAGTCCAATTGGATGCCTCCCTGCTCATTTGCT
TGGTGATATGTGGGGTAGATTTTGGACAAATCTGTACTCTTTGACAGTTC
CCTTTGGACAGAAACCAAACATAGATGTTACTGATGCAATGGTGGACCAG
GCCTGGGATGCACAGAGAATATTCAAGGAGGCCGAGAAGTTCTTTGTATC
TGTTGGTCTTCCTAATATGACTCAAGGATTCTGGGAAAATTCCATGCTAA
CGGACCCAGGAAATGTTCAGAAAGCAGTCTGCCATCCCACAGCTTGGGAC
CTGGGGAAGGGCGACTTCAGGATCCTTATGTGCACAAAGGTGACAATGGA
CGACTTCCTGACAGCTCATCATGAGATGGGGCATATCCAGTATGATATGG
CATATGCTGCACAACCTTTTCTGCTAAGAAATGGAGCTAATGAAGGATTC
CATGAAGCTGTTGGGGAAATCATGTCACTTTCTGCAGCCACACCTAAGCA
TTTAAAATCCATTGGTCTTCTGTCACCCGATTTTCAAGAAGACAATGAAA
CAGAAATAAACTTCCTGCTCAAACAAGCACTCACGATTGTTGGGACTCTG
CCATTTACTTACATGTTAGAGAAGTGGAGGTGGATGGTCTTTAAAGGGGA
AATTCCCAAAGACCAGTGGATGAAAAAGTGGTGGGAGATGAAGCGAGAGA
TAGTTGGGGTGGTGGAACCTGTGCCCCATGATGAAACATACTGTGACCCC
GCATCTCTGTTCCATGTTTCTAATGATTACTCATTCATTCGATATTACAC
AAGGACCCTTTACCAATTCCAGTTTCAAGAAGCACTTTGTCAAGCAGCTA
AACATGAAGGCCCTCTGCACAAATGTGACATCTCAAACTCTACAGAAGCT
GGACAGAAACTGTTCAATATGCTGAGGCTTGGAAAATCAGAACCCTGGAC
CCTAGCATTGGAAAATGTTGTAGGAGCAAAGAACATGAATGTAAGGCCAC
TGCTCAACTACTTTGAGCCCTTATTTACCTGGCTGAAAGACCAGAACAAG
AATTCTTTTGTGGGATGGAGTACCGACTGGAGTCCATATGCAGACGAGCC
CAAATCTTCCGACAAAACTCACACATGCCCACCGTGCCCAGCACCTGAAC
TCCTGGGGGGACCGTCAGTCTTCCTCTTCCCCCCAAAACCCAAGGACACC
CTCATGATCTCCCGGACCCCTGAGGTCACATGCGTGGTGGTGGACGTGAG
CCACGAAGACCCTGAGGTCAAGTTCAACTGGTACGTGGACGGCGTGGAGG
TGCATAATGCCAAGACAAAGCCGCGGGAGGAGCAGTACAACAGCACGTAC
CGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGA
AAACCATCTCCAAAGCCAAAGGGCAGCCCCGAGAACCACAGGTGTACACC
CTGCCCCCATCCCGGGATGAGCTGACCAAGAACCAGGTCAGCCTGACCTG
CCTGGTCAAAGGCTTCTATCCCAGCGACATCGCCGTGGAGTGGGAGAGCA
ATGGGCAGCCGGAGAACAACTACAAGACCACGCCTCCCGTGCTGGACTCC
GACGGCTCCTTCTTCCTCTATAGCAAGCTCACCGTGGACAAGAGCAGGTG
GCAGCAGGGGAACGTCTTCTCATGCTCCGTGATGCATGAGGCTCTGCACA
ACCACTACACGCAGAAGAGCCTCTCCCTGTCTCCGGGTAAATAG

>Sequence ID 21: SI-69R4-human ACE2-ECD-1-740 (TMPRSS2 protease cutting site)-Fc-w2(EU numbering 216-447)-protein sequence

MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADQSIKVRISLKSALGDKAYEWNDNEMYLFRSSVAYA
MRQYFLKVKNQMILFGEEDVRVANLKPRISFNFFVTAPKNVSDIIPRTEV EKAIRMSRSRINDAFRLNDNSLEFLGIQPTLGPPNQPPVSEPKSSDKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSC
SVMHEALHNHYTQKSLSLSPGK

>Sequence ID 22: SI-69R4_human ACE2-ECD-1-740-Fc-w2-DNA sequence

ATGTCAAGCTCTTCCTGGCTCCTTCTCAGCCTTGTTGCTGTAACTGCTGC
TCAGTCCACCATTGAGGAACAGGCCAAGACATTTTTGGACAAGTTTAACC
ACGAAGCCGAAGACCTGTTCTATCAAAGTTCACTTGCTTCTTGGAATTAT
AACACCAATATTACTGAAGAGAATGTCCAAAACATGAATAATGCTGGGGA
CAAATGGTCTGCCTTTTTAAAGGAACAGTCCACACTTGCCCAAATGTATC
CACTACAAGAAATTCAGAATCTCACAGTCAAGCTCCAGCTGCAGGCTCTT
CAGCAAAATGGGTCTTCAGTGCTCTCAGAAGACAAGAGCAAACGGTTGAA
CACAATTCTAAATACAATGAGCACCATCTACAGTACTGGAAAAGTTTGTA
ACCCAGATAATCCACAAGAATGCTTATTACTTGAACCAGGTTTGAATGAA
ATAATGGCAAACAGTTTAGACTACAATGAGAGGCTCTGGGCTTGGGAAAG
CTGGAGATCTGAGGTCGGCAAGCAGCTGAGGCCATTATATGAAGAGTATG
TGGTCTTGAAAAATGAGATGGCAAGAGCAAATCATTATGAGGACTATGGG
GATTATTGGAGAGGAGACTATGAAGTAAATGGGGTAGATGGCTATGACTA
CAGCCGCGGCCAGTTGATTGAAGATGTGGAACATACCTTTGAAGAGATTA
AACCATTATATGAACATCTTCATGCCTATGTGAGGGCAAAGTTGATGAAT
GCCTATCCTTCCTATATCAGTCCAATTGGATGCCTCCCTGCTCATTTGCT
TGGTGATATGTGGGGTAGATTTTGGACAAATCTGTACTCTTTGACAGTTC
CCTTTGGACAGAAACCAAACATAGATGTTACTGATGCAATGGTGGACCAG
GCCTGGGATGCACAGAGAATATTCAAGGAGGCCGAGAAGTTCTTTGTATC
TGTTGGTCTTCCTAATATGACTCAAGGATTCTGGGAAAATTCCATGCTAA
CGGACCCAGGAAATGTTCAGAAAGCAGTCTGCCATCCCACAGCTTGGGAC
CTGGGGAAGGGCGACTTCAGGATCCTTATGTGCACAAAGGTGACAATGGA
CGACTTCCTGACAGCTCATCATGAGATGGGGCATATCCAGTATGATATGG
CATATGCTGCACAACCTTTTCTGCTAAGAAATGGAGCTAATGAAGGATTC
CATGAAGCTGTTGGGGAAATCATGTCACTTTCTGCAGCCACACCTAAGCA
TTTAAAATCCATTGGTCTTCTGTCACCCGATTTTCAAGAAGACAATGAAA
CAGAAATAAACTTCCTGCTCAAACAAGCACTCACGATTGTTGGGACTCTG
CCATTTACTTACATGTTAGAGAAGTGGAGGTGGATGGTCTTTAAAGGGGA
AATTCCCAAAGACCAGTGGATGAAAAAGTGGTGGGAGATGAAGCGAGAGA
TAGTTGGGGTGGTGGAACCTGTGCCCCATGATGAAACATACTGTGACCCC
GCATCTCTGTTCCATGTTTCTAATGATTACTCATTCATTCGATATTACAC
AAGGACCCTTTACCAATTCCAGTTTCAAGAAGCACTTTGTCAAGCAGCTA
AACATGAAGGCCCTCTGCACAAATGTGACATCTCAAACTCTACAGAAGCT
GGACAGAAACTGTTCAATATGCTGAGGCTTGGAAAATCAGAACCCTGGAC
CCTAGCATTGGAAAATGTTGTAGGAGCAAAGAACATGAATGTAAGGCCAC
TGCTCAACTACTTTGAGCCCTTATTTACCTGGCTGAAAGACCAGAACAAG
AATTCTTTTGTGGGATGGAGTACCGACTGGAGTCCATATGCAGACCAAAG
CATCAAAGTGAGGATAAGCCTAAAATCAGCTCTTGGAGATAAAGCATATG
AATGGAACGACAATGAAATGTACCTGTTCCGATCATCTGTTGCATATGCT
ATGAGGCAGTACTTTTTAAAAGTAAAAAATCAGATGATTCTTTTTGGGGA
GGAGGATGTGCGAGTGGCTAATTTGAAACCAAGAATCTCCTTTAATTTCT
TTGTCACTGCACCTAAAAATGTGTCTGATATCATTCCTAGAACTGAAGTT
GAAAAGGCCATCAGGATGTCCCGGAGCCGTATCAATGATGCTTTCCGTCT
GAATGACAACAGCCTAGAGTTTCTGGGGATACAGCCAACACTTGGACCTC
CTAACCAGCCCCCTGTTTCCGAGCCCAAATCTTCCGACAAAACTCACACA
TGCCCACCGTGCCCAGCACCTGAACTCCTGGGGGGACCGTCAGTCTTCCT
CTTCCCCCCAAAACCCAAGGACACCCTCATGATCTCCCGGACCCCTGAGG
TCACATGCGTGGTGGTGGACGTGAGCCACGAAGACCCTGAGGTCAAGTTC
AACTGGTACGTGGACGGCGTGGAGGTGCATAATGCCAAGACAAAGCCGCG
GGAGGAGCAGTACAACAGCACGTACCGTGTGGTCAGCGTCCTCACCGTCC
TGCACCAGGACTGGCTGAATGGCAAGGAGTACAAGTGCAAGGTCTCCAAC
AAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGA
CCAAGAACCAGGTCAGCCTGACCTGCCTGGTCAAAGGCTTCTATCCCAGC
GACATCGCCGTGGAGTGGGAGAGCAATGGGCAGCCGGAGAACAACTACAA
GACCACGCCTCCCGTGCTGGACTCCGACGGCTCCTTCTTCCTCTATAGCA
AGCTCACCGTGGACAAGAGCAGGTGGCAGCAGGGGAACGTCTTCTCATGC
TCCGTGATGCATGAGGCTCTGCACAACCACTACACGCAGAAGAGCCTCTC
CCTGTCTCCGGGTAAATAG

>Sequence ID 23: SI-69R1_huACE2 functional domain (residue:1-615)- 6XHis protein sequence

MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADHHHHHH

>Sequence ID 24: SI-69R1_huACE2 functional domain (residue:1-615)- 6XHis DNA sequence

ATGTCAAGCTCTTCCTGGCTCCTTCTCAGCCTTGTTGCTGTAACTGCTGC
TCAGTCCACCATTGAGGAACAGGCCAAGACATTTTTGGACAAGTTTAACC
ACGAAGCCGAAGACCTGTTCTATCAAAGTTCACTTGCTTCTTGGAATTAT
AACACCAATATTACTGAAGAGAATGTCCAAAACATGAATAATGCTGGGGA
CAAATGGTCTGCCTTTTTAAAGGAACAGTCCACACTTGCCCAAATGTATC
CACTACAAGAAATTCAGAATCTCACAGTCAAGCTTCAGCTGCAGGCTCTT
CAGCAAAATGGGTCTTCAGTGCTCTCAGAAGACAAGAGCAAACGGTTGAA
CACAATTCTAAATACAATGAGCACCATCTACAGTACTGGAAAAGTTTGTA
ACCCAGATAATCCACAAGAATGCTTATTACTTGAACCAGGTTTGAATGAA
ATAATGGCAAACAGTTTAGACTACAATGAGAGGCTCTGGGCTTGGGAAAG
CTGGAGATCTGAGGTCGGCAAGCAGCTGAGGCCATTATATGAAGAGTATG
TGGTCTTGAAAAATGAGATGGCAAGAGCAAATCATTATGAGGACTATGGG
GATTATTGGAGAGGAGACTATGAAGTAAATGGGGTAGATGGCTATGACTA
CAGCCGCGGCCAGTTGATTGAAGATGTGGAACATACCTTTGAAGAGATTA
AACCATTATATGAACATCTTCATGCCTATGTGAGGGCAAAGTTGATGAAT
GCCTATCCTTCCTATATCAGTCCAATTGGATGCCTCCCTGCTCATTTGCT
TGGTGATATGTGGGGTAGATTTTGGACAAATCTGTACTCTTTGACAGTTC
CCTTTGGACAGAAACCAAACATAGATGTTACTGATGCAATGGTGGACCAG
GCCTGGGATGCACAGAGAATATTCAAGGAGGCCGAGAAGTTCTTTGTATC
TGTTGGTCTTCCTAATATGACTCAAGGATTCTGGGAAAATTCCATGCTAA
CGGACCCAGGAAATGTTCAGAAAGCAGTCTGCCATCCCACAGCTTGGGAC
CTGGGGAAGGGCGACTTCAGGATCCTTATGTGCACAAAGGTGACAATGGA
CGACTTCCTGACAGCTCATCATGAGATGGGGCATATCCAGTATGATATGG
CATATGCTGCACAACCTTTTCTGCTAAGAAATGGAGCTAATGAAGGATTC
CATGAAGCTGTTGGGGAAATCATGTCACTTTCTGCAGCCACACCTAAGCA
TTTAAAATCCATTGGTCTTCTGTCACCCGATTTTCAAGAAGACAATGAAA
CAGAAATAAACTTCCTGCTCAAACAAGCACTCACGATTGTTGGGACTCTG
CCATTTACTTACATGTTAGAGAAGTGGAGGTGGATGGTCTTTAAAGGGGA
AATTCCCAAAGACCAGTGGATGAAAAAGTGGTGGGAGATGAAGCGAGAGA
TAGTTGGGGTGGTGGAACCTGTGCCCCATGATGAAACATACTGTGACCCC
GCATCTCTGTTCCATGTTTCTAATGATTACTCATTCATTCGATATTACAC
AAGGACCCTTTACCAATTCCAGTTTCAAGAAGCACTTTGTCAAGCAGCTA
AACATGAAGGCCCTCTGCACAAATGTGACATCTCAAACTCTACAGAAGCT
GGACAGAAACTGTTCAATATGCTGAGGCTTGGAAAATCAGAACCCTGGAC
CCTAGCATTGGAAAATGTTGTAGGAGCAAAGAACATGAATGTAAGGCCAC
TGCTCAACTACTTTGAGCCCTTATTTACCTGGCTGAAAGACCAGAACAAG
AATTCTTTTGTGGGATGGAGTACCGACTGGAGTCCATATGCAGACCATCA
TCACCATCACCAC

>Sequence ID 25: SI-69R10_Human TMPRSS2 protein, His-tagged (106-492)- protein sequence

MYRMQLLSCIALSLALVTNSWKFMGSKCSNSGIECDSSGTCINPSNWCDG
VSHCPGGEDENRCVRLYGPNFILQVYSSQRKSWHPVCQDDWNENYGRAAC
RDMGYKNNFYSSQGIVDDSGSTSFMKLNTSAGNVDIYKKLYHSDACSSKA
VVSLRCIACGVNLNSSRQSRIVGGESALPGAWPWQVSLHVQNVHVCGGSI
ITPEWIVTAAHCVEKPLNNPWHWTAFAGILRQSFMFYGAGYQVEKVISHP
NYDSKTKNNDIALMKLQKPLTFNDLVKPVCLPNPGMMLQPEQLCWISGWG
ATEEKGKTSEVLNAAKVLLIETQRCNSRYVYDNLITPAMICAGFLQGNVD
SCQGDSGGPLVTSKNNIWWLIGDTSWGSGCAKAYRPGVYGNVMVFTDWIY
RQMRADGHHHHHH

>Sequence ID 26: SI-69R10_Human TMPRSS2 protein, His-tagged (106-492)- DNA sequence

ATGTACAGGATGCAACTCCTGTCTTGCATTGCACTAAGTCTTGCACTTGT
CACCAATTCGTGGAAGTTTATGGGTTCTAAATGCTCTAATAGCGGGATAG
AATGTGACAGTAGTGGCACTTGCATTAACCCTTCAAACTGGTGTGATGGG
GTAAGCCATTGCCCCGGGGGGGAAGATGAAAATAGATGTGTTAGGCTCTA
CGGTCCCAACTTTATACTCCAGGTATATTCAAGTCAACGCAAATCATGGC
ATCCAGTGTGTCAAGACGACTGGAACGAAAACTATGGACGCGCTGCATGT
CGAGATATGGGATATAAGAATAACTTCTATAGTTCACAGGGAATCGTAGA
TGACTCTGGATCTACTAGTTTCATGAAACTGAACACCTCTGCCGGAAACG
TAGATATATATAAAAAGCTTTACCACTCCGACGCTTGTAGCTCTAAGGCC
GTAGTTAGCCTCAGATGCATCGCCTGCGGAGTAAACCTCAATTCATCTCG
CCAGAGTAGGATCGTTGGCGGGGAAAGCGCCCTCCCAGGCGCTTGGCCTT
GGCAAGTTTCCCTTCATGTCCAGAATGTTCATGTATGTGGCGGGTCTATA
ATCACCCCAGAATGGATCGTCACAGCTGCCCACTGCGTGGAGAAACCCCT
CAACAATCCTTGGCATTGGACCGCATTTGCCGGAATACTGAGACAATCAT
TTATGTTCTATGGAGCCGGGTACCAAGTCGAAAAGGTCATTTCCCATCCC
AATTATGATTCCAAAACCAAAAACAATGACATAGCCTTGATGAAACTCCA
GAAGCCTTTGACATTTAATGACCTGGTCAAACCAGTGTGCCTCCCAAATC
CTGGAATGATGTTGCAGCCTGAACAGTTGTGCTGGATCAGCGGTTGGGGT
GCTACCGAGGAGAAGGGTAAGACAAGCGAGGTCCTTAACGCTGCAAAGGT
TTTGCTGATAGAAACACAGAGATGTAACAGCCGCTATGTGTACGATAACC
TGATCACCCCAGCTATGATTTGCGCCGGGTTTTTGCAAGGTAACGTCGAT
TCTTGCCAAGGTGACTCAGGCGGCCCTCTTGTTACATCAAAGAACAATAT
ATGGTGGCTTATCGGCGATACATCATGGGGTTCTGGATGTGCTAAAGCCT
ATCGCCCAGGGGTGTATGGCAATGTAATGGTGTTTACAGACTGGATCTAT
AGGCAGATGCGGGCTGACGGTCACCATCATCACCATCACTGA

>Sequence ID 27: IgJ chain

MKNHLLFWGVLAVFIKAVHVKAQEDERIVLVDNKCKCARITSRIIRSSED
PNEDIVERNIRIIVPLNNRENISDPTSPLRTRFVYHLSDLCKKCDPTEVE
LDNQIVTATQSNICDEDSATETCYTYDRNKCYTAVVPLVYGGETKMVETA
LTPDACYPD

>Sequence ID 28: Secretory Component

KSPIFGPEEVNSVEGNSVSITCYYPPTSVNRHTRKYWCRQGARGGCITLI
SSEGYVSSKYAGRANLTNFPENGTFVVNIAQLSQDDSGRYKCGLGINSRG
LSFDVSLEVSQGPGLLNDTKVYTVDLGRTVTINCPFKTENAQKRKSLYKQ
IGLYPVLVIDSSGYVNPNYTGRIRLDIQGTGQLLFSVVINQLRLSDAGQY
LCQAGDDSNSNKKNADLQVLKPEPELVYEDLRGSVTFHCALGPEVANVAK
FLCRQSSGENCDVVVNTLGKRAPAFEGRILLNPQDKDGSFSVVITGLRKE
DAGRYLCGAHSDGQLQEGSPIQAWQLFVNEESTIPRSPTVVKGVAGGSVA
VLCPYNRKESKSIKYWCLWEGAQNGRCPLLVDSEGWVKAQYEGRLSLLEE
PGNGTFTVILNQLTSRDAGFYWCLTNGDTLWRTTVEIKIIEGEPNLKVPG
NVTAVLGETLKVPCHFPCKFSSYEKYWCKWNNTGCQALPSQDEGPSKAFV
NCDENSRLVSLTLNLVTRADEGWYWCGVKQGHFYGETAAVYVAVEERKAA
GSRDVSLAKADAAPDEKVLDSGFREIENKAIQDPR

>Sequence ID 29: Bamlanivimab_Heavy_Chain

QVQLVQSGAEVKKPGSSVKVSCKASGGTFSNYAISWVRQAPGQGLEWMGR
IIPILGIANYAQKFQGRVTITADKSTSTAYMELSSLRSEDTAVYYCARGY
YEARHYYYYYAMDVWGQGTAVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLS
LSPGK

>Sequence ID 30: Bamlanivimab_Light_Chain

DIQMTQSPSSLSASVGDRVTITCRASQSISSYLSWYQQKPGKAPKLLIYA
ASSLQSGVPSRFSGSGSGTDFTLTITSLQPEDFATYYCQQSYSTPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

>Sequence ID 31: Casirivimab_Heavy_Chain

QVQLVESGGGLVKPGGSLRLSCAASGFTFSDYYMSWIRQAPGKGLEWVSY
ITYSGSTIYYADSVKGRFTISRDNAKSSLYLQMNSLRAEDTAVYYCARDR
GTTMVPFDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK

>Sequence ID 32: Casirivimab_Light_Chain

DIQMTQSPSSLSASVGDRVTITCQASQDITNYLNWYQQKPGKAPKLLIYA
ASNLETGVPSRFSGSGSGTDFTFTISGLQPEDIATYYCQQYDNLPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

>Sequence ID 33: Etesevimab_Heavy_Chain

EVQLVESGGGLVQPGGSLRLSCAASGFTVSSNYMSWVRQAPGKGLEWVSV
IYSGGSTFYADSVKGRFTISRDNSMNTLFLQMNSLRAEDTAVYYCARVLP
MYGDYLDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPEAAGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK

>Sequence ID 34: Etesevimab_Light_Chain

DIVMTQSPSSLSASVGDRVTITCRASQSISRYLNWYQQKPGKAPKLLIYA
ASSLQSGVPSRFSGSGSGTDFTLTISSLQPEDFATYYCQQSYSTPPEYTF
GQGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTH
QGLSSPVTKSFNRGEC

>Sequence ID 35: Imdevimab_Heavy_Chain

QVQLVESGGGVVQPGRSLRLSCAASGFTFSNYAMYWVRQAPGKGLEWVAV
ISYDGSNKYYADSVKGRFTISRDNSKNTLYLQMNSLRTEDTAVYYCASGS
DYGDYLLVYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK

> Sequence ID 36: Imdevimab_Light_Chain

QSALTQPASVSGSPGQSITISCTGTSSDVGGYNYVSWYQQHPGKAPKLMI
YDVSKRPSGVSNRFSGSKSGNTASLTISGLQSEDEADYYCNSLTSISTWV
FGGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

>Sequence ID 37: Cilgavimab_Heavy_Chain

EVQLVESGGGLVKPGGSLRLSCAASGFTFRDVWMSWVRQAPGKGLEWVGR
IKSKIDGGTTDYAAPVKGRFTISRDDSKNTLYLQMNSLKTEDTAVYYCTT
AGSYYYDTVGPGLPEGKFDYWGQGTLVTVSSASTKGPSVFPLAPSSKSTS
GGTAALGCLVKDYFPEPVTVSWNSGALTSGVHTFPAVLQSSGLYSLSSVV
TVPSSSLGTQTYICNVNHKPSNTKVDKRVEPKSCDKTHTCPPCPAPEFEG
GPSVFLFPPKPKDTLYITREPEVTCVVVDVSHEDPEVKFNWYVDGVEVHN
AKTKPREEQYNSTYRVVSVLTVLHQDWLNGKEYKCKVSNKALPASIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHY
TQKSLSLSPGK

>Sequence ID 38: Cilgavimab_Light_Chain

DIVMTQSPDSLAVSLGERATINCKSSQSVLYSSNNKNYLAWYQQKPGQPP
KLLMYWASTRESGVPDRFSGSGSGAEFTLTISSLQAEDVAIYYCQQYYST
LTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

>Sequence ID 39: Tixagevimab_Heavy_Chain

QMQLVQSGPEVKKPGTSVKVSCKASGFTFMSSAVQWVRQARGQRLEWIGW
IVIGSGNTNYAQKFQERVTITRDMSTSTAYMELSSLRSEDTAVYYCAAPY
CSSISCNDGFDIWGQGTMVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPEFEGGPSVFLFP
PKPKDTLYITREPEVTCVVVDVSHEDPEVKFNWYVDGVEVHNAKTKPREE
QYNSTYRVVSVLTVLHQDWLNGKEYKCKVSNKALPASIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK

>Sequence ID 40: Tixagevimab_Light_Chain

EIVLTQSPGTLSLSPGERATLSCRASQSVSSSYLAWYQQKPGQAPRLLIY
GASSRATGIPDRFSGSGSGTDFTLTISRLEPEDFAVYYCQHYGSSRGWTF
GQGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTH
QGLSSPVTKSFNRGEC

What is claimed is:

1. A method of preventing, reducing risk of, or treating a virus infection, or preventing or treating a symptom caused by the virus in a subject, said method comprising administering to said subject an effective amount of a fusion protein, wherein the fusion protein comprises a variant angiotensin converting enzyme 2 (ACE2) domain covalently fused to a Fc domain, wherein the variant ACE2 domain comprises a N-terminal deletion, a C-terminal deletion, or both, relative to a full-length wildtype ACE2 having a SEQ ID NO. 1, wherein the variant ACE2 domain has ACE2 activity, wherein the virus comprises a SARS-CoV, SARS-CoV-2, or MERS-CoV, and wherein the symptom comprises Severe Acute Respiratory Syndrome (SARS), Middle East Respiratory Syndrome (MERS), Acute Respiratory Distress Syndrome (ARDS), Pulmonary Arterial Hypertension (PAH), or Coronavirus Disease 2019 (COVID-19).

2. The method of claim 1, wherein the fusion protein comprises an amino acid having at least 98% of sequence identity to SEQ ID NO. 15, 16, 17, or 18.

3. (canceled)

4. The method of claim 1, wherein the dose of the fusion protein administered per day is not more than about 140 mg/Kg body weight.

5. The method of claim 1, wherein the fusion protein is administered twice per day at a dose less than or equal to about 70 mg/Kg body weight.

6. The method of claim 1, wherein the fusion protein is administered through daily infusion or daily intramuscular injections.

7. (canceled)

8. The method of claim 1, wherein the fusion protein is administered as a liquid preparation, wherein the liquid suspension optionally comprises a salt and a surfactant.

9. (canceled)

10. The method of claim 8, wherein the liquid preparation comprises the fusion protein in a concentration from about 5 mg/ml to about 10 mg/ml.

11. The method of claim 1, wherein the administration of the fusion protein prevents or reduces the risk of infection of the subject from the SARS-CoV-2 virus infection.

12. (canceled)

13. The method of claim 1, wherein the administration of the fusion protein prevents or reduces the risk of hospitalization of the subject having the SARS-CoV-2 virus infection.

14-17. (canceled)

18. The method of claim 1, wherein the administration of the fusion protein prevents or reduces the risk of death of the subject having the SARS-CoV-2 virus infection.

19. (canceled)

20. The method of claim 18, wherein the SARS-CoV-2 virus comprises substantially delta strain.

21. The method of claim 18, wherein the SARS-CoV-2 virus comprises a Spike protein mutation, wherein the mutation is configured to increase the binding affinity of the virus to the ACE2 domain.

22. The method of claim 1, wherein the administration of the fusion protein reduces the severity of COVID symptom in the subject having the SARS-Co2-2 virus infection.

23. The method of claim 1, wherein the subject has at least one of risk factor selected from the group consisting of:
an age greater than or equal to 65;
a moderately or severely compromised immune system;
a metabolic syndrome;
being allergic to a COVID vaccine; and
having low or no immune response after receiving a COVID vaccine.

24-25. (canceled)

26. A liquid composition, comprising a fusion protein, wherein the fusion protein comprises a variant angiotensin converting enzyme 2 (ACE2) domain covalently fused to a Fc domain, wherein the variant ACE2 domain comprises a N-terminal deletion, a C-terminal deletion, or both, relative to a full-length wild-type ACE2 having a SEQ ID NO. 1, wherein the variant ACE2 domain has ACE2 activity.

27. The liquid composition of claim 26, having the fusion protein content from about 100 mg to about 10,000 mg per dose.

28. The liquid composition of claim 26, having the fusion protein in a concentration from about 0.5% to about 1% by weight.

29. The liquid composition of claim 26, wherein the variant ACE2 domain comprises an amino acid sequence having at least 98% sequence identity to SEQ ID NO. 3.

30. The liquid composition of claim 26, wherein the Fc domain comprises an amino acid sequence having at least 98% sequence identity to SEQ ID NO. 6.

31. The liquid composition of claim 26, wherein the fusion protein comprises an amino acid sequence having at least 98% of sequence identity to SEQ ID NO. 7, 9, 11, 13, 15, 16, 17, 18, 19, or 21.


SEQUENCE LISTING (28 sequences)

SEQ ID NO: 1 - protein, 805 aa, Artificial Sequence [sequence omitted]

SEQ ID NO: 2 - DNA, 2418 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 3 - protein, 615 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 4 - DNA, 1845 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 5 - protein, 232 aa, Artificial Sequence
  Synthesized
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, Artificial Sequence
  Synthesized
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 847 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 2544 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagcccaaatcttccgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 9 - protein, 844 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - DNA, 2535 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtccaaatatggtcccccgtgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctccaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctgggtaaatga

SEQ ID NO: 11 - protein, 875 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 12 - DNA, 2628 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacagccaggatgtgactgtgccctgcccagttccctcaactccacctaccccatctccctcaactccacctaccccatctccctcatgctgccacccccgactgtcactgcaccgaccggccctcgaggacctgctcttaggttcagaagcgaacctcacgtgcacactgaccggcctgagagatgcctcaggtgtcaccttcacctggacgccctcaagtgggaagagcgctgttcaaggaccacctgagcgtgacctctgtggctgctacagcgtgtccagtgtcctgccgggctgtgccgagccatggaaccatgggaagaccttcacttgcactgctgcctaccccgagtccaagaccccgctaaccgccaccctctcaaaatccggaaacacattccggcccgaggtccacctgctgccgccgccgtcggaggagctggccctgaacgagctggtgacgctgacgtgcctggcacgcggcttcagccccaaggacgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctactga

SEQ ID NO: 13 - protein, 862 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 14 - DNA, 2589 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacagccaggatgtgactgtgccctgccgagttcccccacctcccccatgctgccacccccgactgtcgctgcaccgaccggccctcgaggacctgctcttaggttcagaagcgaacctcacgtgcacactgaccggcctgagagatgcctctggtgccaccttcacctggacgccctcaagtgggaagagcgctgttcaaggaccacctgagcgtgacctctgtggctgctacagcgtgtccagtgtcctgcctggctgtgcccagccatggaaccatggggagaccttcacctgcactgctgcccaccccgagttgaagaccccactaaccgccaacatcacaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccacctatgctgtgaccagcatactgcgcgtggcagccgaggactggaagaagggggaaaccttctcctgcatggtgggccacgaggccctgccgctggccttcacacagaagaccatcgaccgcatggcgggtaaacccacccatatcaatgtgtctgttgtcatggcggaggcggacggcacctgctactga

SEQ ID NO: 15 - protein, 830 aa, Artificial Sequence
  Synthesized
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 827 aa, Artificial Sequence
  Synthesized
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 858 aa, Artificial Sequence
  Synthesized
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 18 - protein, 845 aa, Artificial Sequence
  Synthesized
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 19 - protein, 847 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 2544 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagctccagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagcccaaatcttccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 21 - protein, 972 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 2919 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagctccagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccgagcccaaatcttccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 23 - protein, 621 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADHHHHHH

SEQ ID NO: 24 - DNA, 1863 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccatcatcaccatcaccac

SEQ ID NO: 25 - protein, 413 aa, Artificial Sequence
  Synthesized
MYRMQLLSCIALSLALVTNSWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADGHHHHHH

SEQ ID NO: 26 - DNA, 1242 bp, Artificial Sequence
  Synthesized
atgtacaggatgcaactcctgtcttgcattgcactaagtcttgcacttgtcaccaattcgtggaagtttatgggttctaaatgctctaatagcgggatagaatgtgacagtagtggcacttgcattaacccttcaaactggtgtgatggggtaagccattgccccgggggggaagatgaaaatagatgtgttaggctctacggtcccaactttatactccaggtatattcaagtcaacgcaaatcatggcatccagtgtgtcaagacgactggaacgaaaactatggacgcgctgcatgtcgagatatgggatataagaataacttctatagttcacagggaatcgtagatgactctggatctactagtttcatgaaactgaacacctctgccggaaacgtagatatatataaaaagctttaccactccgacgcttgtagctctaaggccgtagttagcctcagatgcatcgcctgcggagtaaacctcaattcatctcgccagagtaggatcgttggcggggaaagcgccctcccaggcgcttggccttggcaagtttcccttcatgtccagaatgttcatgtatgtggcgggtctataatcaccccagaatggatcgtcacagctgcccactgcgtggagaaacccctcaacaatccttggcattggaccgcatttgccggaatactgagacaatcatttatgttctatggagccgggtaccaagtcgaaaaggtcatttcccatcccaattatgattccaaaaccaaaaacaatgacatagccttgatgaaactccagaagcctttgacatttaatgacctggtcaaaccagtgtgcctcccaaatcctggaatgatgttgcagcctgaacagttgtgctggatcagcggttggggtgctaccgaggagaagggtaagacaagcgaggtccttaacgctgcaaaggttttgctgatagaaacacagagatgtaacagccgctatgtgtacgataacctgatcaccccagctatgatttgcgccgggtttttgcaaggtaacgtcgattcttgccaaggtgactcaggcggccctcttgttacatcaaagaacaatatatggtggcttatcggcgatacatcatggggttctggatgtgctaaagcctatcgcccaggggtgtatggcaatgtaatggtgtttacagactggatctataggcagatgcgggctgacggtcaccatcatcaccatcactga

SEQ ID NO: 27 - protein, 159 aa, Artificial Sequence
  Synthesized
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 28 - protein, 585 aa, Artificial Sequence
  Synthesized
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPR